SEQUENCE LISTING (91 sequences)

SEQ ID NO: 1 - DNA, 1011 bp, Plodia interpunctella
  source 1..1011: mol_type = genomic DNA
atgacccctaaccctgactctaccgaggtgctgtctaaggactctggccctccacctctcgagaagctggtggctccccaggctggaccccgaaagtaccagttcgtgtacctgaacatgatgacccacggctactggcacctgtctgccatctacggcctgtacctgtctttcaccaccgccaagtgggctaccctgatcttcgccctgttcatcttcgtgatctctaagatctctgtgaccgccggctctcaccgactgtgggctcaccgatcttacaaggccaagctgcccctcgagatcctgctgatcatcatgaactctatcgccttccaggacaccgccttcacctgggctcgagatcaccgactccaccaccgacacgccgacaccgacgctgaccctcacaactctacccgaggcttcttctactctcacatcggctggctgttcgtgaagaagcaccccgaggtcgtcgcccgaggcaagaccattcctatcgaggacctgaagaagaacaagctgctgcagttccagcgaaagtacgctttccctctgatgggcaccctgtgtttcttcatgcccaccttcgtgcccgtgtacttctggggcgagtctgtgatcaccgcctggcacatcaacctgctgcgatacgtgtgcaacctgaacgccatcttctctgtgaactctgccgctcacatgttcggctacaagccctacgacaagaagatcgcctctgtccagaacctgcctgtgaccttcgtgaccctcggcgagggctaccacaactaccaccacacttacccctgggactaccgagccgccgagctgggcaacaacaagtacaacctgactaccaagttcatcgactttttcgcctggatcggctgggcctacgacctcaagaccgtgcctgaggacaccatcaagatgcgaatggcccgatctggcgacggcaccgacctctggggcctcgagcgagatgagctggtgaacggaatcaacaagtag

SEQ ID NO: 2 - protein, 336 aa, Plodia interpunctella
MTPNPDSTEVLSKDSGPPPLEKLVAPQAGPRKYQFVYLNMMTHGYWHLSAIYGLYLSFTTAKWATLIFALFIFVISKISVTAGSHRLWAHRSYKAKLPLEILLIIMNSIAFQDTAFTWARDHRLHHRHADTDADPHNSTRGFFYSHIGWLFVKKHPEVVARGKTIPIEDLKKNKLLQFQRKYAFPLMGTLCFFMPTFVPVYFWGESVITAWHINLLRYVCNLNAIFSVNSAAHMFGYKPYDKKIASVQNLPVTFVTLGEGYHNYHHTYPWDYRAAELGNNKYNLTTKFIDFFAWIGWAYDLKTVPEDTIKMRMARSGDGTDLWGLERDELVNGINK

SEQ ID NO: 3 - DNA, 1146 bp, Drosophila virilis
  source 1..1146: mol_type = genomic DNA
atgcctccaaacgccatcacttctccctctaacgtgctggccacctgtcctgaggtcgagcagctgccctctggcgagtgcgcctctaccgcctcttctaagcagaccggcgtgctgttcgagggcgacgccgacaccgccgactgcgccctggacgtggacgtgaagaagctgaagaaggccgagaagcgaaagctgaagctggtgtggcgaaacatcatgctgttcggctacctgcacctggccgccgtgtacggcggatacctgatgctgacccaggccaagtgggctaccgtggtgttctctttcttcctgtacaccgccggcatgatcggcatcaccggcggagcccaccgactgtgggctcaccgatcttacaaggctaagtggcccctgcgagtgatcctggtgaccttcaacaccattgccttccaggacgccgccttccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacgtgaccgacctgcgagccgaccgaatcctgatgttccagctgaagcactacttcgtgctgatgcccctggcctgcttcattctgcccaccatcattcccatgatctgctggaacgagtctctgctgtgctcctggttcgtggccaccatgttccgatggtgcttccagctcaacatgacctggctggtgaactctgccgctcacaagttcggcggacgaccctacgacaagaacatcaacccctctcagtctccctacgtgtctgccttcaccttcggcgaaggctggcacaactaccaccacgtgttcccttgggactacaagaccgccgagtggggccgatactctctgaacatgactaccgccttcatcgactttttcgccaagatcggctgggcctacgagctgaagtctgtggctcccgagactatcgagcgacgagtgcgacgaaccggcgacggcacccacgagctgtggggctggggcgacaaggacctgaccgctgaggacgcccagcacgtcctgttcgtggacaagaaggctgcctag

SEQ ID NO: 4 - protein, 381 aa, Drosophila virilis
MPPNAITSPSNVLATCPEVEQLPSGECASTASSKQTGVLFEGDADTADCALDVDVKKLKKAEKRKLKLVWRNIMLFGYLHLAAVYGGYLMLTQAKWATVVFSFFLYTAGMIGITGGAHRLWAHRSYKAKWPLRVILVTFNTIAFQDAAFHWARDHRVHHKFSETDADPHNATRGFFFSHVGWLLCKKHPDVVAKGKSLDVTDLRADRILMFQLKHYFVLMPLACFILPTIIPMICWNESLLCSWFVATMFRWCFQLNMTWLVNSAAHKFGGRPYDKNINPSQSPYVSAFTFGEGWHNYHHVFPWDYKTAEWGRYSLNMTTAFIDFFAKIGWAYELKSVAPETIERRVRRTGDGTHELWGWGDKDLTAEDAQHVLFVDKKAA

SEQ ID NO: 5 - DNA, 1086 bp, Drosophila melanogaster
  source 1..1086: mol_type = genomic DNA
atggctccctactctcgaatctaccaccaggacaagtcgtcccgagagactggcgtgctgttcgaggacgacgcccagaccgtggactctgacctgaccaccgaccgattccagctgaagcgagccgagaagcgacgactgcccctggtgtggcgaaacatcatcctgttcgccctggtgcacctggccgctctgtacggcctgcactctatcttcacccgagccaagctggccaccactctgttcgctgccggcctgtacatcatcggcatgctgggcgtgaccgctggcgcccaccgactgtgggctcaccgaacctacaaggccaagtggcccctgcgactgctgctggtgatcttcaacaccattgccttccaggacgccgtgtaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggccgaggcctggacctgtctgacctgcgagctgaccccatcctgatgttccagcgaaagcactactacattctgatgcccctggcctgcttcgtgctgcccaccgtgattcccatggtgtactggaacgagactctggcctcttcctggttcgtggccaccatgttccgatggtgcttccagctcaacatgacctggctggtgaactctgccgctcacaagttcggcaaccgaccttacgacaagactatgaaccccactcagaacgccttcgtgtctgccttcaccttcggcgaaggctggcacaactaccaccacgcattcccttgggactacaagaccgccgagtggggctgctactctctgaacatcaccaccgccttcatcgacctgttcgctaagatcggctgggcctacgacctcaagaccgtggctcccgacgtgatccagcgacgagtgctgcgaaccggcgacggctctcacgagctgtggggctggggcgacaaggacctgaccgctgaggacgcccgaaacgtcctgctggtggacaagtctcgataa

SEQ ID NO: 6 - protein, 361 aa, Drosophila melanogaster
MAPYSRIYHQDKSSRETGVLFEDDAQTVDSDLTTDRFQLKRAEKRRLPLVWRNIILFALVHLAALYGLHSIFTRAKLATTLFAAGLYIIGMLGVTAGAHRLWAHRTYKAKWPLRLLLVIFNTIAFQDAVYHWARDHRVHHKYSETDADPHNATRGFFFSHVGWLLCKKHPDIKEKGRGLDLSDLRADPILMFQRKHYYILMPLACFVLPTVIPMVYWNETLASSWFVATMFRWCFQLNMTWLVNSAAHKFGNRPYDKTMNPTQNAFVSAFTFGEGWHNYHHAFPWDYKTAEWGCYSLNITTAFIDLFAKIGWAYDLKTVAPDVIQRRVLRTGDGSHELWGWGDKDLTAEDARNVLLVDKSR

SEQ ID NO: 7 - DNA, 1149 bp, Drosophila grimshawi
  source 1..1149: mol_type = genomic DNA
atgcctccaaacgctatctcttctcctcctgacgctctgcccacctgtcctgaggaccaggtcctgtctggctctcagtctggcgctggcggagcctctaagcagaccggcgtgctgttcgaggccgacgccgacaccaacgacggcggcctgaccatggacatcacccagttcaagaacgccgagaagcgaaagctgaagtgggtgtggcgaaacatcgtcctgttcgcctacgtccacgtggccgctctgtacggcggctacctgctggtgacccaggccaagtgggccactatcgtgttctctatgttcctgtactctgccggcatgatcggcatcaccggcggagcccaccgactgtgggctcaccgatcttacaaggctaagtggcccctgcgagtgatcctggtggccttcaactctatcgccttccaggacgccgcctaccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacctgtctgacctgcgagccgaccgagtgctgatgttccaaaagaagcactacttcgttctgatgcccctggcctgcttcattctgcctacctgcattcccatgctgtgctggaacgagtctctgctgtgctctgtgctggtgcccaccatcttccgatggtgcctgcagctgaacatgacctggctggtgaactctgccgctcacaagttcggcggacgaccctacgacaagaacatcaacccttctcagtctccctacgtgtctctgttcaccctcggcgaaggctggcacaactaccaccacgtgttcccttgggactacaagaccgccgagtggggcaactactctctcaacatgaccaccgccttcattgacctgttcgctaagatcggctgggcctacgacctgaagtctgtggtgcccgagactgtcgagcgacgagtgcgacgaaccggcgacggctctcacgagctgtggggctggggcgacaaggacctgacacctgaggacggccagtctgtgctgcacgtgctggacaagaagtctaactag

SEQ ID NO: 8 - protein, 416 aa, Drosophila grimshawi
MFSEYYVYRRKLLGISSVQIARQIDRLIKPSTLKMPPNAISSPPDALPTCPEDQVLSGSQSGAGGASKQTGVLFEADADTNDGGLTMDITQFKNAEKRKLKWVWRNIVLFAYVHVAALYGGYLLVTQAKWATIVFSMFLYSAGMIGITGGAHRLWAHRSYKAKWPLRVILVAFNSIAFQDAAYHWARDHRVHHKFSETDADPHNATRGFFFSHVGWLLCKKHPDVVAKGKSLDLSDLRADRVLMFQKKHYFVLMPLACFILPTCIPMLCWNESLLCSVLVPTIFRWCLQLNMTWLVNSAAHKFGGRPYDKNINPSQSPYVSLFTLGEGWHNYHHVFPWDYKTAEWGNYSLNMTTAFIDLFAKIGWAYDLKSVVPETVERRVRRTGDGSHELWGWGDKDLTPEDGQSVLHVLDKKSN

SEQ ID NO: 9 - DNA, 981 bp, Amyelois transitella
  source 1..981: mol_type = genomic DNA
atggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 10 - protein, 351 aa, Amyelois transitella
MPPQGQDRESWVLYETDDKTQDGGTHVVPPSAEKRVWKIVWRNVIAFALLHIGGVYGAYLFLFKAMWLTDLFAVFLYLCSGLGVTAGAHRLWAHKSYKARLPLRLLLTVFNTIAFQDAVIDWARDHRLHHKYSETDADPHNATRGFFFSHIGWLLVRKHPQIKEKGPTIDLNDLRADPVLHFQKKYYLYLMPLACFVMPTLVPTLWGESLWNAYFVCAIFRYIYVLNVTWLVNSAAHAWGSKPYDKNINPVETKPVSLVVLGEGFHNYHHTFPWDYKTAELGDYSLNFSKLFIDAMAKIGWAYDLKTVSPDVIEKRVKRTGDGSHHVWGWDDKDVPVEEKEEATIFNPSKD

SEQ ID NO: 11 - DNA, 1059 bp, Amyelois transitella
  source 1..1059: mol_type = genomic DNA
atggctccaaatgccacagatgctaatggggttctgtttgagacggatgcagctaccccagacctggccctacctcacgcccctgtacagcaagcggacaattaccctaaaaaatatgtgtggagaaatataatattatttgcctatctccacattgctgcactctatggtggctacttgttcttgttccatgctaaatggcagacagatatatttgcttacattctgtatgtgatgtcaggcttgggaataacggcaggagcccatcgtctgtgggcccacaagtcctacaaagctaaatggccactcagattgatacttgtcatctttaacactctggcattccaggactctgcaatcgactggtctcgcgaccaccgaatgcaccacaaatactcggaaactgatgctgatccccacaacgcgacccgcggtttcttcttctcccacattggctggctgctggttaggaagcaccctgaacttaagaggaaaggcaaaggtctggaccttagtgacttatatgctgaccccattttgagattccaaaagaagtactacttgatcctgatgcccctcacttgcttcgtgttacctacggtcatccccgtgtattactggggcgagacttggaccaacgccttcttcgtggccgctcttttccggtatgccttcatccttaacgtcacctggctggtcaattctgctgctcacaagtggggcgataagccttacgatagaaacatcaagccttcggaaaatatttccgtctccatgttcgcgctcggagaaggcttccacaactaccaccatactttcccctgggactacaagaccgcggaacttggcaacaacatgctcaatttcacgactaacttcatcaatttctttgctaaaatcggttgggcgtacgatctgaaaactgtttccgatgaaattgttaggagccgcgctaagagaacaggagatggttctcaccatctatggggctggggtgataaggatcactctcgtgaggaaatggccgccgctataaggatacacccgaaagatgactag

SEQ ID NO: 12 - protein, 352 aa, Amyelois transitella
MAPNATDANGVLFETDAATPDLALPHAPVQQADNYPKKYVWRNIILFAYLHIAALYGGYLFLFHAKWQTDIFAYILYVMSGLGITAGAHRLWAHKSYKAKWPLRLILVIFNTLAFQDSAIDWSRDHRMHHKYSETDADPHNATRGFFFSHIGWLLVRKHPELKRKGKGLDLSDLYADPILRFQKKYYLILMPLTCFVLPTVIPVYYWGETWTNAFFVAALFRYAFILNVTWLVNSAAHKWGDKPYDRNIKPSENISVSMFALGEGFHNYHHTFPWDYKTAELGNNMLNFTTNFINFFAKIGWAYDLKTVSDEIVRSRAKRTGDGSHHLWGWGDKDHSREEMAAAIRIHPKDD

SEQ ID NO: 13 - DNA, 1533 bp, Saccharomyces cerevisiae
  source 1..1533: mol_type = genomic DNA
atgcccacctctggcaccaccatcgagctgattgacgatcagttccccaaggacgactctgcctcttctggcatcgtggacgaggtggacctgaccgaggccaacatcctggccaccggcctgaacaagaaggctccccgaatcgtcaacggcttcggctctctgatgggctctaaggaaatggtgtctgtcgagttcgacaagaagggcaacgagaagaagtctaacctggaccgactgctcgagaaggacaaccaggaaaaggaagaggccaagaccaagatccacatctctgagcagccctggactctgaacaactggcaccagcacctgaactggctgaacatggtgctggtctgcggcatgcccatgatcggctggtacttcgccctgtctggcaaggtgcccctgcacctcaacgtgtttctgttctctgtgttctactacgccgtcggcggcgtgtctatcaccgccggctaccaccgactgtggtcccaccgatcttactctgctcactggcccctgcgactgttctacgccatcttcggctgcgcctctgtcgagggctctgccaagtggtggggccactctcaccgaatccaccaccgatacaccgacactctgcgagatccctacgacgcccgacgaggcctgtggtactctcacatgggctggatgctgctgaagcccaaccctaagtacaaggcccgagccgacatcaccgacatgactgacgactggaccatccgattccagcaccgacattacatcctgctgatgctgctcaccgccttcgtgatccccactctgatctgcggctacttcttcaacgactacatgggcggcctgatctacgccggcttcatccgagtgttcgtgattcagcaggctaccttctgtatcaactctctggcccactacatcggcacccagcctttcgacgaccgacgaacccctcgagacaactggatcaccgccattgtgaccttcggcgagggctaccacaacttccaccacgagttccccaccgactaccgaaacgccatcaagtggtatcagtacgaccccaccaaggtgattatctacctgacctctctggtcggcctggcctacgacctgaagaagttctctcagaacgccatcgaggaagccctgatccagcaagagcagaagaagatcaacaagaagaaggcaaagatcaactggggacccgtgctgaccgacctgcctatgtgggacaagcagaccttcctggccaagtccaaggaaaacaagggcctcgtcatcatctctggcattgtgcacgacgtgtctggctacatttctgagcaccccggtggcgagactctgatcaagaccgctctcggcaaggacgccaccaaggccttctctggcggagtgtaccgacattctaacgccgctcagaacgtgctggccgacatgcgagtggccgtgatcaaggaatctaagaactctgccatccgaatggcctctaagcgaggcgagatctacgagactggcaagttcttttag

SEQ ID NO: 14 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSLAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 15 - DNA, 1449 bp, Yarrowia lipolytica
  source 1..1449: mol_type = genomic DNA
atggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttag

SEQ ID NO: 16 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 17 - DNA, 1062 bp, Spodoptera litura
  source 1..1062: mol_type = genomic DNA
atggctcccaacatctctgaggacgtgaacggcgtgctgttcgagtctgacgccgctactcccgacctggctctggcccgacctcctgtgcagaaggccgacaacaagcccaagcagctggtgtggcgaaacatcatcctgttcgcctacctgcacctggccgctctgtacggcggctacctgttcctgttctctgccaagtggcagaccgacatcttcgcctacatcctgtacgtgatctctggcctgggcatcaccgctggcgcccaccgactgtgggctcacaagtcttacaaggctaagtggcccctgaaggtgatcctgatcatcttcaacaccgtggccttccaggacgccgccatggactgggcccgagatcaccgaatgcaccacaagtactctgagactgacgctgaccctcacaacgctacccgaggcttcttcttctctcacatcggctggctgctggtgcgaaagcaccccgacctgaaggaaaagggcaagggcctcgacatgtctgacctgctggctgaccccgtgctgcgattccagaagaagtactacctgctgctgatgcccctggcctgcttcgtcatgcccaccgtgattcccgtgtacctgtggggcgagacttggaccaacgccttcttcgtggccgccatgttccgatacgccttcattctgaacgtgacctggctggtgaactctgctgcccacaagtggggcgacaagccctacgacaagtctatcaagccctctgagaacatgtctgtggccatgttcgccctcggcgagggattccacaactaccaccacacattcccttgggactacaagaccgccgagttcggcaacaacaagctgaacttcactaccgccttcatcaactttttcgccaagatcggctgggcttacgacatgaagaccgtgtctgaggacatcgtgaagaaccgagtgaagcgaaccggcgacggctctcaccacctctggggctggggcgacgagaaccagcctaaggaagagatcgaggccgccatccgaatcaaccccaaggacgactaa

SEQ ID NO: 18 - protein, 353 aa, Spodoptera litura
MAPNISEDVNGVLFESDAATPDLALARPPVQKADNKPKQLVWRNIILFAYLHLAALYGGYLFLFSAKWQTDIFAYILYVISGLGITAGAHRLWAHKSYKAKWPLKVILIIFNTVAFQDAAMDWARDHRMHHKYSETDADPHNATRGFFFSHIGWLLVRKHPDLKEKGKGLDMSDLLADPVLRFQKKYYLLLMPLACFVMPTVIPVYLWGETWTNAFFVAAMFRYAFILNVTWLVNSAAHKWGDKPYDKSIKPSENMSVAMFALGEGFHNYHHTFPWDYKTAEFGNNKLNFTTAFINFFAKIGWAYDMKTVSEDIVKNRVKRTGDGSHHLWGWGDENQPKEEIEAAIRINPKDD

SEQ ID NO: 19 - DNA, 1368 bp, Helicoverpa armigera
  source 1..1368: mol_type = genomic DNA
atggtggtcctgacctctaaggagactaagccctccgtggccgagttctacgctggcaagtctgtcttcatcaccggcggaaccggtttcctgggcaaggtcttcattgagaagctgctgtactcctgtcccgacatcggcaacatctacatgctgatccgagagaagaagggactgtctgtgtccgagcgaattaagcacttcctggacgaccccctgttcacccgactgaaggagaagcgacccgccgacctggagaagatcgtgctgattcccggagacatcaccgctcccgacctgggtattacctctgagaacgagaagatgctgatcgagaaggtgtctgtcatcattcactccgccgctaccgtcaagttcaacgagcccctgcccaccgcctggaagatcaacgtggagggaacccgaatgatgctggctctgtctcgacgaatgaagcgaattgaggtcttcatccacatttccaccgcctacaccaacaccaaccgagaggtggtggacgagatcctgtaccctgctcctgctgacattgaccaggtgcaccgatacgtcaaggacggtatctctgaggaagagactgagaagattctgaacggccgacccaacacctacaccttcaccaaggccctgaccgagcacctggtggctgagaaccaggcttacgtgcccaccatcattgtccgaccctccgtggtcgccgctatcaaggacgagcccattaagggatggctgggtaactggtacggagctaccggactgaccgtgttcaccgctaagggtctgaaccgagtcatctacggccactcttccaacatcgtggacctgattcccgtggactacgtcgccaacctggtcattgccgctggcgctaagtcttccaagtccaccgagctgaaggtgtacaactgttgctcttccgcctgcaaccccatcaccattggaaagctgatgtctatgttcgccgaggacgctatcaagcagaagtcctacgctatgcccctgcccggttggtacatcttcaccaagtacaagtggctggtcctgctgctgaccattctgttccaggtcatccccgcctacattaccgacctgtaccgacacctgatcggcaagaacccccgatacattaagctgcagtctctggtcaaccagacccgatcttccattgacttcttcacctctcactcctgggtcatgaaggctgaccgagtccgagagctgttcgcctctctgtcccccgctgacaagtacctgttcccctgtgaccccaccgacatcaactggacccactacattcaggactactgctggggagtgcgacacttcctggagaagaagtcctacgagtag

SEQ ID NO: 20 - protein, 455 aa, Helicoverpa armigera
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 21 - DNA, 1365 bp, Spodoptera exigua
  source 1..1365: mol_type = genomic DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggaaaagcgacccggcgacctggacaagattgtgctgatccccggcgacgtgaccgtgcctggcctgggcatctctgacgagaacgaggccatcctgatcgacaaggtgtctgtggtgatccactctgccgccaccgtgaagttcaacgagcccctcgagactgcctggaacgtgaacgtcgagggcacccgaatgatcatggccctgtctcgaaagatgaagcgaatcgagatcttcatccacatctctaccgcctacaccaacaccaaccgagccgtggtggacgaggtgctgtaccctcctcctgccgacatcaacgaggtgcaccagtacgtgaagaacggcatcaccgaggaagagactgagaagatcctgaacggacgacccaacacctacaccttcaccaaggctctgaccgagcacctggtggccgagaaccaggcctacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccagtcctctcacgtggtggatctgattcccgtggactacgtggccaacctggtgatcgtggctggcgccaagacctaccgatctaacgaggtgactatctacaactcttgttcttcttcttgcaaccccatcaccatggaacgactggtgggcctgttcattgacgacaccgtcaagcacaactcttacgtgatgcccctgcctggctggtacgtgtactctaactaccgatggctggtgtacctggtgaccatcatcttccagatgatccccgcctacctggccgacatcggccgacgactgctgggcaagaaccctcggtactacaagctgcagtctctggtggctcagacccaagaggccgtgcacttcttcacctctcacacctgggagatcaagtctaagcgaacctctgagctgttcgcctctctgtctcataccgaccagcgaatcttcccatgcgacgccaagaagatcgactggaccgactacatcaccgactactgctctggcgtgcgacagttcctggaaaagaagaagtag

SEQ ID NO: 22 - protein, 454 aa, Spodoptera exigua
MVVLTSKEKSNMSVADFYAGKSVFITGGTGFLGKVFIEKLLYSCPDIDKIYMLIREKKGQSIRERLTKIVDDPLFNRLKEKRPGDLDKIVLIPGDVTVPGLGISDENEAILIDKVSVVIHSAATVKFNEPLETAWNVNVEGTRMIMALSRKMKRIEIFIHISTAYTNTNRAVVDEVLYPPPADINEVHQYVKNGITEEETEKILNGRPNTYTFTKALTEHLVAENQAYMPTIIVRPSIVGAIKDDPIRGWLANWYGATGLSVFTAKGLNRVIYGQSSHVVDLIPVDYVANLVIVAGAKTYRSNEVTIYNSCSSSCNPITMERLVGLFIDDTVKHNSYVMPLPGWYVYSNYRWLVYLVTIIFQMIPAYLADIGRRLLGKNPRYYKLQSLVAQTQEAVHFFTSHTWEIKSKRTSELFASLSHTDQRIFPCDAKKIDWTDYITDYCSGVRQFLEKKK

SEQ ID NO: 23 - DNA, 1380 bp, Spodoptera exigua
  source 1..1380: mol_type = genomic DNA
atgacctaccgacagatcaacgagttcgacgccgagaagttcaccgccgctgctgtgcccacctcttacgtgtctgtgcccgacttctacgccggcaagtctatcttcattaccggcggcaccggattcctcggcaaggtgttcctcgagaagctgctgtactcttgcaaggacgtcgagactgtgtacatcctgatccgagagaagaagggcaagacccctcagcagcgagtcgaggacctgttcaacaagcccatcttctcgcgactgaagcaaaaggactctcagtgcatgaagaaggtgaccgccatcatcggcgacctgtctgagcccggcctgggcatctctaaggacgacgaggaactgctgctgcaaaaggtgtctgtggtgttccacgtggccgccaacgtgcagttctacaaggaattcaaggaaatcatcaacaccaacgtcggaggcaccaagtacgtgctgcagctgtgccagcgaatcaaggacatcaaggccttcgtgcacatctctaccgcctactgccacaccgaccagaaggtgctggaagaacgaatctaccctcctcctgccgaactgtctgaggtgctgaagttcctgcagcagccccagcacgacaagaagcagatcaaggaactgtttaagaagcagcccaactcttacaccttcgccaaggctctggccgagacttacattgccgagaactgcggacgagtgcccaccatcatcattcgaccctctattatctctgcctctctgaaggaacccctgcctggctgggtcgactcttggaacggcgccaccggcctgatcaccgcctcttacaacggcgctaaccgagtgctgctcggcgaaggcggcaacttcctggacctgattcctgtggacttcgtggccaacctggccatcgtggccgctgccaagtgcacctcttcgctgaaggtgtacaactgctgttcttctggctgcaaccctctgactctgaagcagctggtgtctcacatgaacaacgtgggcttcgacaagaacgtgtctatcatctttaccaacaacaaggcctctctgtctaccctgacattcttcctgcagaccactccttcttttaccgccgacatgttcctgcgagtgaccggaaagtctccccgatatatgaagatccagtctaagctgaccattgctcgaaacgccctgaacttcttcacctgtcactcttgggtgatgaaggccgacaactctcgacgactgtacgcttctctgtctctgcacgaccgacacactttcccttgcgaccccaccgacatcgactggaagaagtacatcaccatctacatcgagggaatcaaccagttcctgatgaagaagcgatcttag

SEQ ID NO: 24 - protein, 459 aa, Spodoptera exigua
MTYRQINEFDAEKFTAAAVPTSYVSVPDFYAGKSIFITGGTGFLGKVFLEKLLYSCKDVETVYILIREKKGKTPQQRVEDLFNKPIFSRLKQKDSQCMKKVTAIIGDLSEPGLGISKDDEELLLQKVSVVFHVAANVQFYKEFKEIINTNVGGTKYVLQLCQRIKDIKAFVHISTAYCHTDQKVLEERIYPPPAELSEVLKFLQQPQHDKKQIKELFKKQPNSYTFAKALAETYIAENCGRVPTIIIRPSIISASLKEPLPGWVDSWNGATGLITASYNGANRVLLGEGGNFLDLIPVDFVANLAIVAAAKCTSSLKVYNCCSSGCNPLTLKQLVSHMNNVGFDKNVSIIFTNNKASLSTLTFFLQTTPSFTADMFLRVTGKSPRYMKIQSKLTIARNALNFFTCHSWVMKADNSRRLYASLSLHDRHTFPCDPTDIDWKKYITIYIEGINQFLMKKRS

SEQ ID NO: 25 - DNA, 1365 bp, Spodoptera litura
  source 1..1365: mol_type = genomic DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggcaagatcattctgatccccggcgacatcaccgtgcctggcctgggcatctctgaggaaaacgagactatcctgatcgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactaccgatggctggtgttcctggtgaccctgatcttccaggtgatccccgcctacctgggcgacatcggccgacgactgctgggaaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 26 - protein, 454 aa, Spodoptera litura
MVVLTSKEKSNMSVADFYAGKSVFITGGTGFLGKVFIEKLLYSCPDIDKIYMLIREKKGQSIRERLTKIVDDPLFNRLKDKRPGDLGKIILIPGDITVPGLGISEENETILIEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYRWLVFLVTLIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 27 - DNA, 1608 bp, Plodia interpunctella
  source 1..1608: mol_type = genomic DNA
atgcgactggtgccctctgctctggacgtggtgctggccgccgagcgacgactggtccccatccacgaggctaccctgaagggcgactctgaggtgcagaagttctacgccggctctgtggtcctggtgaccggcggctctggcttcctgggcaagcagctgatcgagaagctgttccgaacctgcgacgtgaagaagctgtacatcctgctgcgagccaagaagggcaagtctatcggcgagcgactggactacattctgcgaaaccccgtgtacgacaccctgcgagagactcgaccctggttcgccgaccgaatcgaggccgtcgagggcgacattgcctcttaccgactgggcgtcgccgaccatatgtgggaccgactggtggacgaggtgaccgtggtgttccacggcgctgccaccatcaacttcgctgaccctctgcagctggccaccaacatcaacgtgaagggcaccctcgagatgctcgagttcggcaaggcctgcaagaacctccgatcttacgtgcacatctctaccgcctacaaccaggctctgaagaacaacgtgtcttgcgacatcctggaagagttccccaaggctcccctgtctcctaccactctggtggacctggccaaccgaaccgagactaaggtgctggacgagatgttccacaagtacctggcacctgagtaccccaacacctacgcctacaccaaggccgtggccgaagaggccgtgcgaaccatggccggcgacctgcctatctgcgtggtgcgaccctctatcgtgatccccgcctacactgagccctctgccggctgggtcgacaagaactctatctacggcgcctctggcatcatcatgggcctcggcctgggcgtgattcacaccctgatctctgaccccgacatcaagatcgacatcatccccgtggacatcgtgaacaacgccgtgatcgtggccggatgggagactgcccgacgacgagagcagggcgagactgacaccaagatctacaccgtgggatcttctaaccgaaaccacatcacctggcgatttctgaccaacgtgttcgacaacgagggccgaaagcacctgtcctctcaggccgtgtggtacgccttcgccgtgcagaccaagtctaagcacctctacctggctctggcctggctgctgcacttcatccccggctacatcgtggacggcgtgctgatgatccgaggcaagaagccccaggtttctaagatctaccgactgctgaacactatggccaccatcttctcgttcttcaccctccgaggctggaacttcaaggacgacaacctgctgcagctgtacaagtctctgtctaagaccgaccagcagatcttcaacatggacattgctaccctggacatgaaggaagtgatctacctctggcacattggcctgcgacgattctacctgaaggacaagctgaacgaccaagagaagtctatgaagaagcagttcttcctgcgaatcatcacctacaccttcattcctatctacctgtacttcctgttcaagatctcttgcttcgtgttctacggcctgtactacctgatcttctcttacctgatttga

SEQ ID NO: 28 - protein, 535 aa, Plodia interpunctella
MRLVPSALDVVLAAERRLVPIHEATLKGDSEVQKFYAGSVVLVTGGSGFLGKQLIEKLFRTCDVKKLYILLRAKKGKSIGERLDYILRNPVYDTLRETRPWFADRIEAVEGDIASYRLGVADHMWDRLVDEVTVVFHGAATINFADPLQLATNINVKGTLEMLEFGKACKNLRSYVHISTAYNQALKNNVSCDILEEFPKAPLSPTTLVDLANRTETKVLDEMFHKYLAPEYPNTYAYTKAVAEEAVRTMAGDLPICVVRPSIVIPAYTEPSAGWVDKNSIYGASGIIMGLGLGVIHTLISDPDIKIDIIPVDIVNNAVIVAGWETARRREQGETDTKIYTVGSSNRNHITWRFLTNVFDNEGRKHLSSQAVWYAFAVQTKSKHLYLALAWLLHFIPGYIVDGVLMIRGKKPQVSKIYRLLNTMATIFSFFTLRGWNFKDDNLLQLYKSLSKTDQQIFNMDIATLDMKEVIYLWHIGLRRFYLKDKLNDQEKSMKKQFFLRIITYTFIPIYLYFLFKISCFVFYGLYYLIFSYLI

SEQ ID NO: 29 - DNA, 1569 bp, Plodia interpunctella
  source 1..1569: mol_type = genomic DNA
atggtggcccgacgagactctgaggccgtgcctctgattccccagttctacgccggacgatctatcttcatcaccggcgccaccggcttcatgggcaaggtgctgatcgagcgaatcctggccacctgtcctgacgtggaccgactgtacctgctggtgcgacacaagaaggacgtttctcccgagaagcgactgcagcagctgatcgactctcccgtgttcgaccccgtgcgagctactgaccccgctcagctggacaagctgtctatgctggccggcgacatcaccaagcctgacctgggcatgtctcaagagtctctgctgcgactgcacgacgtgtctgtggtgttccactctgccgccactctgaagttcgacgaggccatgaagatggccgtcgagcagaacgtgctgtctgtgatccgactgctcgagatctgcgaccgactgcctaacatgcaggccttcgtgcacgtgtctaccgcctactctaacgccgagctgcgagaggtcgaggaacacgtgtaccctccaaagatctcgctccagcagctcctggccgtgtctgagtcgctgcccgaggacctgatgatggaaatcacccctaagttcatctctcccaagcctaacacctacaccttcaccaaggccatggctgagcacgccgtgcagcagcacggaaacaagggctacgcctgcgccatcttccgacctaccatcgtgatctcttccgtgaagcacccctatcctggctggatcgagaacctgaacggcccctctggcgtgatcgccgctgccggcaagggcctgctgcatgtgttctgctgcaaccccgacgctgccgccgacatgctgcccgtggacattgccatcgacaccctgctggccgtggcctgggagactgccattgacaagtctaaggaagtgcgagtctacaactgctctacccgagagaaccccaccgactggcgaggcttcgagcaggctctgcgacggtatatcgtcgagcaccctctggaccgagcctactggtatccctccggcgcctctgtgggcaacacctacgctcagaaggccatcgagctgctcacccagactgtgcccctgcacctggtggcctacaccattcgactgctgggcatcaagacccagctgaacctgatcaccgtgtctcaccgactgcaggctatgaacaaggtcctgaagttcttcgcccagcgagagtgggtgtttcacaacgagaacgtgcgacgactgcgagcccgaatctcgccccaggacgccaagatctacaacctggatcctaactctttcgactgggacgagcactactgcaacttcatcaagggcacccgaaagtacctgctcaaggaaaaggaacaggacatcgaggaagcccgaaagcacatcaagaagatgttctacgtccactactccctgctgctgttcgtggtggtgctgctcctgcgattcgccctgaacaactccttcgtgcgacagttcgtgtacggcgtggtgcgaaccctgaccgccgtggctggcgccgtgttctctcgaatctctgcctag

SEQ ID NO: 30 - protein, 522 aa, Plodia interpunctella
MVARRDSEAVPLIPQFYAGRSIFITGATGFMGKVLIERILATCPDVDRLYLLVRHKKDVSPEKRLQQLIDSPVFDPVRATDPAQLDKLSMLAGDITKPDLGMSQESLLRLHDVSVVFHSAATLKFDEAMKMAVEQNVLSVIRLLEICDRLPNMQAFVHVSTAYSNAELREVEEHVYPPKISLQQLLAVSESLPEDLMMEITPKFISPKPNTYTFTKAMAEHAVQQHGNKGYACAIFRPTIVISSVKHPYPGWIENLNGPSGVIAAAGKGLLHVFCCNPDAAADMLPVDIAIDTLLAVAWETAIDKSKEVRVYNCSTRENPTDWRGFEQALRRYIVEHPLDRAYWYPSGASVGNTYAQKAIELLTQTVPLHLVAYTIRLLGIKTQLNLITVSHRLQAMNKVLKFFAQREWVFHNENVRRLRARISPQDAKIYNLDPNSFDWDEHYCNFIKGTRKYLLKEKEQDIEEARKHIKKMFYVHYSLLLFVVVLLLRFALNNSFVRQFVYGVVRTLTAVAGAVFSRISA

SEQ ID NO: 31 - DNA, 1362 bp, Trichoplusia ni
  source 1..1362: mol_type = genomic DNA
atgatcgccgtgacctctcacgagaacgaggcctctatcgtggacttctacgagggcaagtctgtgttcattaccggcggcaccggattcctcggcaaggtgttcatcgagaagctgctgtactgctgtcccggcctggtgaagatctacatgctgatccgagagaagaagggcctgaccatcaaggaacgaattgaccgattcgtggacgactctctgttcgaccgactcaagacccagcgacctaaggacctggacaagatcgtgctgatccccggcgacatcactgccccttctctgggcatctctcaagagcaagagaacatcctgatcgaagaggtgtctgtgatcatccactctgccgccaccatcaagttcaacgcccctctggaagaggcctggtctatcaacgtggacggcacccgacgaatcctggacctgtgccgaaacatgaagaaggtcgaggttttcctgcacatctctaccgcctacaccaacatcaaccgaaacgtgatcgaggaagtgctctaccctcctccagccgacattaaggaactggaaaagctggacaagcacaacgtgaccgagaagcagaccatgaagctgctcaacggcttccccaacacctacaccttcaccaagtctctgaccgagcacctggtggccgagaactgcgcctacatgcccaccatcatcgtgcgaccctctgtggtggccgccgtgctcgaggaccccatcaagggctggctggaaaactggtacggcgccaccggcatctccgtgttcaccgccaagggactgaaccgagtgttcctgggccacaagaacattattgtggacgtgattcccgccgactacgtggccaacctggtgatcattgccggcgctcgaaacaacaagtccaaggaactcaagatctataactgctgttcttctggctgcaaccctctgaccatcggcaacctgctgggcgccttcatcgacgacgccatccgacacaagacctacgctatgcccctgcctggctggttcatcttcacccgataccgactggtggctaccatcgtgaccttcatcttccaggtggtgcccgcctacctggccgacctgtaccgacgaattatcggcaagaagcccaagtacaccaagctgctctacctggtggtgcagacccgagtggccctggacttcttctcctctaactcttgggtgatgaaggccgaccgaatgcgagagctgtacgcctctctgtctaacaacgacaagcgaatgttcccttgcgaccccactcacatcaactggcccgagtacctgtctgactacggcgctggcgtgcgacgattcctcgagaagcgaaactag

SEQ ID NO: 32 - protein, 453 aa, Trichoplusia ni
MIAVTSHENEASIVDFYEGKSVFITGGTGFLGKVFIEKLLYCCPGLVKIYMLIREKKGLTIKERIDRFVDDSLFDRLKTQRPKDLDKIVLIPGDITAPSLGISQEQENILIEEVSVIIHSAATIKFNAPLEEAWSINVDGTRRILDLCRNMKKVEVFLHISTAYTNINRNVIEEVLYPPPADIKELEKLDKHNVTEKQTMKLLNGFPNTYTFTKSLTEHLVAENCAYMPTIIVRPSVVAAVLEDPIKGWLENWYGATGISVFTAKGLNRVFLGHKNIIVDVIPADYVANLVIIAGARNNKSKELKIYNCCSSGCNPLTIGNLLGAFIDDAIRHKTYAMPLPGWFIFTRYRLVATIVTFIFQVVPAYLADLYRRIIGKKPKYTKLLYLVVQTRVALDFFSSNSWVMKADRMRELYASLSNNDKRMFPCDPTHINWPEYLSDYGAGVRRFLEKRN

SEQ ID NO: 33 - DNA, 60 bp, synthetic construct
  misc_feature 1..60: note = Primer PR25093
  source 1..60: mol_type = other DNA
acgtgcaacgcttatggtctcagcgggaagactttacgacgcaactaacatgaatgaata

SEQ ID NO: 34 - DNA, 54 bp, synthetic construct
  misc_feature 1..54: note = Primer PR25094
  source 1..54: mol_type = other DNA
cacgcgatatggtctcacagagaagacttcagacgatttgtcttagaggaacgc

SEQ ID NO: 35 - DNA, 54 bp, synthetic construct
  misc_feature 1..54: note = Primer PR25314
  source 1..54: mol_type = other DNA
aggccactatggtctcacagagaagacttcagacgatttgtcttagaggaacgc

SEQ ID NO: 36 - DNA, 55 bp, synthetic construct
  misc_feature 1..55: note = Primer PR25381
  source 1..55: mol_type = other DNA
cgtgcgatatggtctcagcgggaagactttacgacgcaactaacatgaatgaata

SEQ ID NO: 37 - protein, 547 aa, Drosophila grimshawi
MGDSTLKLPGAAAAPLKPPAVAALSVSSLQLPSSAPQKLTSAASNGNVSGSATGNPRNKCALKPGYSLMSWIRLCNSGADLSGTAGRVVPVSRTELARHNQVNDAWMAIRGKVFNVTRYMDYHPGGVDELMRGVGRDATKLFDEVHAWVNYPQLLGKCYVGPLKDTVASKKATDAELLRLPPPSAPAELKPRFDWIQQRRELTLYFYTRRWANPGVLLKRKTAEELEVRVQLAEIWHLYNFKLHSGIIWPPKALRVSPETGKIELVLSKQEEQPWSSYGSHEASKTSGAPEEELLDFMVQHCSHFNHDSFELCLESVEQELLLHLPVGYHLDIEVPHNGERVQRSYTPVPHNCLLAEAYSSSSSPNSQRFLIKHYENGIVSSQLNQLESGGNLQLTLPRGNFNLAQLEAHRHILLLAAGSGLTPMLSLLRPLLERHTNRIERLQLLYFNKTQADIWWKDKLLALQSSDERFLCQHILSQEVEEQPPTPNQNTRICPELLAPLFEQQQPLGERFSYVAICGPTGFNTAALDSLRRLEFKLTNVHVFQG

SEQ ID NO: 38 - protein, 535 aa, Drosophila melanogaster
MSDPSLKPPASNQPLRPPQLMAPAGSGGGLQLPVTAPQKLNSNGSSASGSATGNPRNKCALKPGYSLMNWIRLCNSGADLSGTGGRLVPVSRTELARHNKIDDAWMAIRGRVFNVTRYMDFHPGGVDELMRGVGRDATKLFDEVHAWVNYPQLLGKCYVGPLKDNETKPAKESPQITNVILQPPEIVPRFDWIQQRSELTLIFYTRSLANPGLVVRRKDLQQLSVRVLVQHNWHSFDFQLTNNVEWPPKLAKIGSETGKIELVFVKEEAEPWPTYGTHVSNRLDSSRLHDETFEYEVVHSKDFNHDSFELCLQSVGQKVLMVLPAGYHVDIEVPLEGRVIQRSYTPVDHTYLRLENIRSSRSECLHFLIKRYPNGPVSSHLQKLETGSRVHWSAPRGSFQLSDLTAHRNILLLAAGSGLTPILSLIQPILKRNTNRIESLQLLYFNKTNEDIWLKEKLHELHTDDERFSCTNYVSQSEDNPQRIALELLAPLFQKNQPERCTYVLICGPSGFNTAALDILSQLDVKANQIHVFRG

SEQ ID NO: 39 - protein, 521 aa, Homo sapiens
MLNVPSQSFPAPRSQQRVASGGRSKVPLKQGRSLMDWIRLTKSGKDLTGLKGRLIEVTEEELKKHNKKDDCWICIRGFVYNVSPYMEYHPGGEDELMRAAGSDGTELFDQVHRWVNYESMLKECLVGRMAIKPAVLKDYREEEKKVLNGMLPKSQVTDTLAKEGPSYPSYDWFQTDSLVTIAIYTKQKDINLDSIIVDHQNDSFRAETIIKDCLYLIHIGLSHEVQEDFSVRVVESVGKIEIVLQKKENTSWDFLGHPLKNHNSLIPRKDTGLYYRKCQLISKEDVTHDTRLFCLMLPPSTHLQVPIGQHVYLKLPITGTEIVKPYTPVSGSLLSEFKEPVLPNNKYIYFLIKIYPTGLFTPELDRLQIGDFVSVSSPEGNFKISKFQELEDLFLLAAGTGFTPMVKILNYALTDIPSLRKVKLMFFNKTEDDIIWRSQLEKLAFKDKRLDVEFVLSAPISEWNGKQGHISPALLSEFLKRNLDKSKVLVCICGPVPFTEQGVRLLHDLNFSKNEIHSFTA

SEQ ID NO: 40 - protein, 671 aa, Spodoptera litura
MADSPEMEQKPTISNTHRVSLPTVLCTPESTDDNSDISIKVNSIISSDSKMSLPSNLGTPTSPLRLSSPLSLPSSPCVKNSPQSTNSLPNPNPKPMTSSTASNKTVVNSGSATGNPRNKCALQPGHSLMDWIRLGNSGKDLTGVGGRMRPVTPAELSTHNTTNDAWLAIRGRVYNITHYLPYHPGGPEELMRGAGMDATQLFDKVHPWVNYDSLLAKCLVGPLRFDLPDAEELFDTSNPSPKSDRLREPSKAQELVRKSMENLANCITPVRKKITKGDDNSKGSPPSKIMQSLVQSADLPMSISRRAASSPVKKTDKSPDSPLPLRHDWIQTSTKLTISVYTGHLANPGGCARITEGFLLIEVATNGWLRTLKIKPEAKLKEPLQLRVFAESGKIEITALKAEPSVWKGCGEVTLGAASRVSSPRTVECRVMEVSRVSHDTSLLSVCPRAGPVVVPLGHHVRVHRRVEGSECVRSYTPVGEGWGPPDGTEIFSALRLAVKRYDSGVLSPHLTALKVGDLITLSGPYGNFQLQKLKTVKSLYLIAAGTGITPMLGLIKFMMSRSNPRCERTHLIFFNKTEADILFLENFEEMAKEDDRLKITHVLSDASSSWSGHKGRIRDELLTEILGQVKCDDKCSHYSCLCGPTEFTYAGLDLLKKHGFKDDCIHAFMG

SEQ ID NO: 41 - protein, 679 aa, Cydia pomonella
MADSPPEDNTPTRNGSLKISLPTVLCTPESSENSDILCKTKTFISSESKLSLPSNLGTPTSPLRLSSPLSLPTSPTPYMKITQPQPEKTVAATQMNSPPTTIVQQTGPKQPLVNTGSATGNPRNKCALQPGHSLMDWIRLGNSGKDLTGVGGRMRPVSPDELAAHNTRNDAWLAIRGRVYNITHYLPYHPGGPEELMRGAGIDATQLFDKVHPWVNYDSLLAKCLVGPLRLDRPDAEELFGPSTPSPKSENRLREPSKAQELVRKSMENLANCITPVRKKMSAKSEENIKGSPPSKIMQSLIQSSDLPVSISRRAAASPVKTSEKSDIAPSPLRFDWIQTSTKLTLSIYTGPLANPGGCARIIEGILFVEVATNGWVRTLKLVPEAKLLESLQLRVFAESGKIEVTAHKAEPGVWKTCGEAMAGAASRVSTPRSVSCRVARMLRVSHDTVLLSLAAPAQALVAPLGHHVRVHRTISDVECVRSYTPVGEGWGPDAERDSAIHLAVKGYDTGALSPYLTALQPGDEVTVSGPYGNFQLHTLKGVKEIYFVAAGTGITPMLGLLKFMLPRSNPRCERIHLLFFNKTEEDILFRENFEEIAREDDRFTVTHVLSDAGPSWSGHKGRVTTNLLTQVIGKSFLKCRDQCTHFACVCGPTEFTHTAIDLLKKLDMKDSCIHAFMG

SEQ ID NO: 42 - DNA, 1572 bp, Plodia interpunctella
  source 1..1572: mol_type = genomic DNA
atggctccctctgtgcgagagttctacaagggcaagaacatcttcattaccggcggcaccggattcctcggcaaggccctgatcgagaagctgctgcgatcttgccccgacgtgtctaccatctacctgctgctccgacagaagaagggcctgtcctctgacgagcgactgcgagagctgtgcaacaacaaggtgttcgacgtggtgcgagagcagaaccccgaggtgttcaagaagctgcgactgatctctggcgacattctcgaggaccgactgggcctgtctgccgaggacgagcgagaactgcgagaacgatgcaacatcgtgttccactctgccgcctgcgtgcgattcgaccaaaagctgaaggacgccgtgcacatgaacaccaccggcactctgcgagtgctcgagctggccgagactatggaacatctcgaggtgtttctgcacctgtctaccgcctactgccgatgccacctggacgtgctggaagagaagtcttaccccgctgtgcacgaccctcgacgagtgatggaactggtcgagtggatggacgacgacacccagaactacctcgagcctaagctgatctcttctgagcccaacacctactcttacaccaaggccatcaccgagcagctggtggaagagtaccaagagaagttccccattgccattgctcgaccctctatcgtgatcgccgcctggaaggaacccattcctggctgggtcgacaacatgaacggccccaccggcctgctgatcggctctggcaagggcgccgtgcgatctatgcactgcgagcccgccttcgccgccgacgtgctgcccgtggacgtgaccgtgaacacctgtctgctggccgcctgtgccaccgccaccgaccgacctaaggaagtggtgttctacaacgtgaccctgtctgaggtgatcaagattacctggggcgagatcatcgagctgggcaagaagtggctggctatctaccctcacactctggccctgtggcaccccggtggcaccatcaagtcttactactttcagcaccagctgtgcgtgctgttttaccatctgatccccgcctacctggtggacggactgctgttcctgctgggaaagaaaaccttcctggtcaacgcccagaagcgaatctctcacggactgtctgtgctgcagtactacaccaccaagcagtggcacttcgtcaacaccaacttccgatctctgcgaaaccgaatctctaaggaagatgacgagatcttcttcactgacgtgtcgcccctggactgcgaggacttcctgaagaactacatcctgggagcccgatattacgtgtgcaaggaagatccctctactctgccccgagccagaaagctgtaccgaatccgattcatcgtggaccagatcttccgactcctgttctacttcctgatcttctggctgctgtactcctacaaggacttcttcgtgacctctgttgagaagctggactctgccctgaagtctctgacccctatcaacgtgaaggccgacgtgtgtgagctgaacgaactgaacgaggtgctgtag

SEQ ID NO: 43 - protein, 523 aa, Plodia interpunctella
MAPSVREFYKGKNIFITGGTGFLGKALIEKLLRSCPDVSTIYLLLRQKKGLSSDERLRELCNNKVFDVVREQNPEVFKKLRLISGDILEDRLGLSAEDERELRERCNIVFHSAACVRFDQKLKDAVHMNTTGTLRVLELAETMEHLEVFLHLSTAYCRCHLDVLEEKSYPAVHDPRRVMELVEWMDDDTQNYLEPKLISSEPNTYSYTKAITEQLVEEYQEKFPIAIARPSIVIAAWKEPIPGWVDNMNGPTGLLIGSGKGAVRSMHCEPAFAADVLPVDVTVNTCLLAACATATDRPKEVVFYNVTLSEVIKITWGEIIELGKKWLAIYPHTLALWHPGGTIKSYYFQHQLCVLFYHLIPAYLVDGLLFLLGKKTFLVNAQKRISHGLSVLQYYTTKQWHFVNTNFRSLRNRISKEDDEIFFTDVSPLDCEDFLKNYILGARYYVCKEDPSTLPRARKLYRIRFIVDQIFRLLFYFLIFWLLYSYKDFFVTSVEKLDSALKSLTPINVKADVCELNELNEVL

SEQ ID NO: 44 - DNA, 1536 bp, Plodia interpunctella
  source 1..1536: mol_type = genomic DNA
atggactttttcgaccgagacatgtccaacgagcccaccattcctgagttctacaagggcaagaccattttcatcaccggcggctctggcttcatgggcaaggtgctgatcgagaagctgctgtactcttgccccgacatcgaccgaatctacctgctgctgcgatctaagcgaggcgtgggcgccgagtctcgactggcccagatctactcctctgcctgcttcgaccgactgcgagcccagaagcctaccattttctctgagaaggtgttctacgtcggcggcgacgtgatggaacccgctctgggcatctctggcgaggatcgagccctgctggtgaaccgagtgaacatcattttccacgtggccgcctctgtgcgattcgacgaccctctgcctttcgccgtgaagctgaacctcggcggcaccaagcaggtcgtcgagttcgccaaggaagtgaaggacctgctggtcatggtgcacgtgtctacctcttactctaacaccaaccgagacaccatcgaggaagtgatgtaccctcctcacgccgactggcgagacactatccagatctgcgagactctggacgagcacaccatcaaggtgctcacccctaagtacctgggcgagctgcccaacacctacacctttaccaagcagctggccgagcacgccgtgcgagagctgcgagccagagtgcccctggtgatggtgcgaccctctatcgtggtgtcctctgtgaaggaacccatgcctggctggatcgagaacttcaacggacccgccggaatcctggtggcctgcggcaagggcatcatgcgatctctgtacaccgagcctgacctgatcgccgactacatgcccgtggacatctctatcaagtctttcatcgtggccgcttggatgcgaggcaccctgaagctgcagcccaccgacgacatccccatctacaactgctgtgccggcgacctgaacaacatcaccatgggagagctgatggacatcggcaagaagatggccgccatcatgcccctgaacgacatgctgtggaacgtcggaggcaccattaccacctctaagaccatccactacattaaggtgctgctgctccactgtctgcccgctctgttcgtggacgccctgctgtggctgatgggacgaaagcccatgctgctgaaggtgcagcgacgaatctacattgccaacctggctctgcagtactacatcactaagcagtggaactttaacaacaagaacttcgtcaagctgcgatcccgaatcaaggccgaggaccacgaccacttctactacgagatggaaaccgtggaccgaaacgagtacttcaagcagtgctgtatcggcggacgaaagttcctgctcaaggaaaaggacgaggacctgcctcgagccaaggctcacttcacccgaatgctgatcctggacaagatggtgcagtgcattttctacggcaccctgttctggtggctggtcaactcctcttacgtgcagtctctgatgtctttctttgtgaactga

SEQ ID NO: 45 - protein, 511 aa, Plodia interpunctella
MDFFDRDMSNEPTIPEFYKGKTIFITGGSGFMGKVLIEKLLYSCPDIDRIYLLLRSKRGVGAESRLAQIYSSACFDRLRAQKPTIFSEKVFYVGGDVMEPALGISGEDRALLVNRVNIIFHVAASVRFDDPLPFAVKLNLGGTKQVVEFAKEVKDLLVMVHVSTSYSNTNRDTIEEVMYPPHADWRDTIQICETLDEHTIKVLTPKYLGELPNTYTFTKQLAEHAVRELRARVPLVMVRPSIVVSSVKEPMPGWIENFNGPAGILVACGKGIMRSLYTEPDLIADYMPVDISIKSFIVAAWMRGTLKLQPTDDIPIYNCCAGDLNNITMGELMDIGKKMAAIMPLNDMLWNVGGTITTSKTIHYIKVLLLHCLPALFVDALLWLMGRKPMLLKVQRRIYIANLALQYYITKQWNFNNKNFVKLRSRIKAEDHDHFYYEMETVDRNEYFKQCCIGGRKFLLKEKDEDLPRAKAHFTRMLILDKMVQCIFYGTLFWWLVNSSYVQSLMSFFVN

SEQ ID NO: 46 - DNA, 1035 bp, Plodia interpunctella
  source 1..1035: mol_type = genomic DNA
atggtgcccaacaagggatcttctgtggtgctgaccgagaacgctgagccccagttcaagaagctggtcgctccccaggctggaccccgaaagtaccagatcgtgtaccgaaacctgctgaccttcggctactggcacctgtctgccatctacggcctgtacctgtgcttcacctctgccaagtgggctactattatcttcgccttcttcctgtacatgttcgccgagatcggcatcaccgctggcgcccaccgactgtggacccaccgagcctacaaggccaagctgcccctgcagatcctgctgatcatcatgaactctatcgccttccaggacaccgctctgacctggtgccgagatcaccgaatgcaccaccgatactctgacaccgacgctgaccctcacaacgctacccgaggcttcttctactctcacgtcggctggctgctggtgaagaagcaccccgaggtcaagacccgaggcaagtacattcccctggacgacctgcgaaacaaccccgtgctgcgattccagaagaagtacgctatccccttcgtgggcaccctgtgctttctgatgcccaccttcgtgcccgtgtacttctggggcgagtctatctctaccgcctggcacatcaacctgctgcgatacgtgaccaacctgaacgtgaccttcctggtgaactctgccgctcacctgatcggcaacaagccctacgaccgaactctggcctctgtgcagaacatccccgtgtctatcgccaccttcggcgagggcttccacaactaccaccacacttacccctgggactaccgaaccgccgagctgggcaacaaccgactgaacgtcactaccaagttcatcgactttttcgcctggctcggctgggcctacgacctcaagaccgtgcctcaagaggccgtcgagaagcgaatggcccgaaccggcgacggcaccgacctgtggggctacggcgaaaagaagcccgtcaaggacgttaaggacaactctgacgagacttactacgacttttag

SEQ ID NO: 47 - protein, 344 aa, Plodia interpunctella
MVPNKGSSVVLTENAEPQFKKLVAPQAGPRKYQIVYRNLLTFGYWHLSAIYGLYLCFTSAKWATIIFAFFLYMFAEIGITAGAHRLWTHRAYKAKLPLQILLIIMNSIAFQDTALTWCRDHRMHHRYSDTDADPHNATRGFFYSHVGWLLVKKHPEVKTRGKYIPLDDLRNNPVLRFQKKYAIPFVGTLCFLMPTFVPVYFWGESISTAWHINLLRYVTNLNVTFLVNSAAHLIGNKPYDRTLASVQNIPVSIATFGEGFHNYHHTYPWDYRTAELGNNRLNVTTKFIDFFAWLGWAYDLKTVPQEAVEKRMARTGDGTDLWGYGEKKPVKDVKDNSDETYYDF

SEQ ID NO: 48 - DNA, 1959 bp, Plodia interpunctella
  source 1..1959: mol_type = genomic DNA
atggaagagaactctaacaccaacggcgacggcaagctggacgtgcgacagctctctaagaagcgagaggccgactggcccgctgtgctgttcttcatccacatccatctgctgtctctgtacggcctgtggctgctgctgtacgaggtgcagtggctgaccgtgctgttcctgatgtttctgacctctatcgccgtgctgggcgtgaccaccggcgctcaccgactgtgggcccatcgagcctaccgagcctctaccggcctgcgaatcaccctgatgctgttccagaccatgaccggccagggctctgtgtacgagtgggtgcgacaccaccgactgcaccaccagctgttcgccaccgacaacgacccctacgactacaacaagggcttcctgtactctcacattctgacccgactctggaagctgtctccccagcaagagaagctgcgagagtctatcgacatgtctgacctcgagaaggacggcgtggtgatgttccaaaagaagatgtactggctgctctacctggtcctgttcctgctgctgcccctgaacgctcccctcgagtactggaacgacaccatcctgtctgccgtgttcgtgatcggcttcctgcgatacggcatcgtgctgcacgcctcttggctggtcgagtctggcatctctgtgtggggactgaagcccggccagaagtctcctcctgactctaactctgtgtttatcctgtctaagaccttctggccccactaccactacctggtgccttacgattacaagtctggcgagtacggcacctacgacaccggctgttctaccgccttcatccgagtgtgggccgctctgggcctcgccaccaacctgcgaaccgtggacacccctactctgcagaaggccctggccatcaccgccaagactcagctgcctctgcagacctgcctggaccagctggccgacaagaccaaccgagaggaccattacctgaagcgaggcatgggcgccaaggtgtctcgaaccgacttcgagtgggtctacaccgaggaaccccacgcctctcgacgaaagatcatcctggaaaagtaccccgagatcaagcagctgtttggctacgaccctctgttcaagtgggtcgtgaccggcatggtgctgacccagatcgccatgctgcctctcgtccagcacctgtcttggcccgtgatgctgctgctcgcctactgcttcggcggcgtgatcaaccactctctgatgctggccatccacgagatcgcccacaacctggccttcggccacgctcgacccctgcacaaccgactgttcggcttcttcgccaacctgcctatcggactgcccgtgtctatctctttcaagaagtaccacctcgagcaccaccgataccagggcgacgaggtgatcgacaccgacctgcctactctgatcgaggccaagctgttcaacaccactggcggaaagctgctgtggctgtttctgcagcccttcttctacaccgtgcgacccctgatggtgcgacctaagcctcctactcctctcgagctgatcaacctggtgatccagctgtttttcgacgccgtggtggtgaagctgtacggctggaaggtgctggcctacctcgtgctgggatctctgatggccatgggagtgcaccccgtcgccggccacttcatctctgagcactacatgttccgaaagggcttcgagacttactcttactacggacccctgaactggattaccttcaacgtgggctaccacaacgagcaccatgacttccccgccgtgcctggccgacgactgcccgaggtgcgacgaatcgccaaggaattctacgacgatctgccccagcaccactcttggtctgccgtcctgtacgacttcgtgatggaccccgacatcggaccctacgctcgaatcaagcgaaagcaccacggcctggactcttag

SEQ ID NO: 49 - protein, 652 aa, Plodia interpunctella
MEENSNTNGDGKLDVRQLSKKREADWPAVLFFIHIHLLSLYGLWLLLYEVQWLTVLFLMFLTSIAVLGVTTGAHRLWAHRAYRASTGLRITLMLFQTMTGQGSVYEWVRHHRLHHQLFATDNDPYDYNKGFLYSHILTRLWKLSPQQEKLRESIDMSDLEKDGVVMFQKKMYWLLYLVLFLLLPLNAPLEYWNDTILSAVFVIGFLRYGIVLHASWLVESGISVWGLKPGQKSPPDSNSVFILSKTFWPHYHYLVPYDYKSGEYGTYDTGCSTAFIRVWAALGLATNLRTVDTPTLQKALAITAKTQLPLQTCLDQLADKTNREDHYLKRGMGAKVSRTDFEWVYTEEPHASRRKIILEKYPEIKQLFGYDPLFKWVVTGMVLTQIAMLPLVQHLSWPVMLLLAYCFGGVINHSLMLAIHEIAHNLAFGHARPLHNRLFGFFANLPIGLPVSISFKKYHLEHHRYQGDEVIDTDLPTLIEAKLFNTTGGKLLWLFLQPFFYTVRPLMVRPKPPTPLELINLVIQLFFDAVVVKLYGWKVLAYLVLGSLMAMGVHPVAGHFISEHYMFRKGFETYSYYGPLNWITFNVGYHNEHHDFPAVPGRRLPEVRRIAKEFYDDLPQHHSWSAVLYDFVMDPDIGPYARIKRKHHGLDS

SEQ ID NO: 50 - DNA, 867 bp, Plodia interpunctella
  source 1..867: mol_type = genomic DNA
atgatccacctgtctaagcaggtttctcagtgccgacaccgagagacttccgagatcctgatctgcctgtactggaagatccccactctgatcttcgcctacgtgctgggcttcgcctctatcctgggcatcaccgctggcgcccaccgactgtggtcccacaaggcctacaaggccaagactcccctgcaggtcctgctggtgttcttcttcctgctgtctggccagcgatctatcctgacctgggtcgagaaccaccgaatccaccacaagtactctgacaccgacgctgaccctcacaacgctacccgaggcttcttcttctctaccctcggctggctggccgtcgagccccatcctgaggtgatgaagcagcgaaagctgaccgacctgtctgacctgctgcagatccccgccgtgaagttccagcacgagtacgtcgagattctgatcaccctggtggccctgatctttcctacctacattcccaccttttggggcgagtctctgtacgtgtctttcttcgtgaactacttccgactgctggtggtgctgaccatctctggcttcatcaactctatcgcccacctgtggggcaacaagcccatcgacaagaccgccgtgggcacccagttcaccttcatcggcgccctggctctcggcgagggcttccacaactaccaccacgtgttcccttacgactaccgaacctctgaggtgctggacaccaagtacaacttcaccgccgtgttcatcgacctgatggccaagatcggctgggcctacgacctgaaggaagtgcccgaggaaaccgtgcgatctcgagtgctgaagaagggcgacggctctgacaagtactacctgtag

SEQ ID NO: 51 - protein, 288 aa, Plodia interpunctella
MIHLSKQVSQCRHRETSEILICLYWKIPTLIFAYVLGFASILGITAGAHRLWSHKAYKAKTPLQVLLVFFFLLSGQRSILTWVENHRIHHKYSDTDADPHNATRGFFFSTLGWLAVEPHPEVMKQRKLTDLSDLLQIPAVKFQHEYVEILITLVALIFPTYIPTFWGESLYVSFFVNYFRLLVVLTISGFINSIAHLWGNKPIDKTAVGTQFTFIGALALGEGFHNYHHVFPYDYRTSEVLDTKYNFTAVFIDLMAKIGWAYDLKEVPEETVRSRVLKKGDGSDKYYL

SEQ ID NO: 52 - DNA, 1146 bp, Plodia interpunctella
  source 1..1146: mol_type = genomic DNA
atggctcccgctcagtctaacgtgatcgcccaggaccagatgcaccgacagctgaagatgcgactgattccccgagatcaggactgcgacaagtgcgacaacacctgtgacaacaacaactctgtgctgcacaactactctggctctgagaagatcaactctgaggccgagttcgacctgtctaagtacgaggccatcgagttcagaccccgaattcgatggcccgacctgaccgtgcagatctctctgcacctggtggctatctacggcctgttcctgatcctgaccaaccagatcaagttctacaccctgctgttcgtgctgggcaccatctacacctctggcttcggcatcaccgctggcgtgcaccgactgtggtcccaccgagcctaccgagccagactgcccctgcgaatcctgctggccttcctgttcaccatcaccggccagcgagacatctacacttgggccctcgaccaccgagtgcaccacaagtacaccgagactgtggctgaccctcacgacgtgcgacgaggcttctggttcgcccacgtcggctggctggtgctgacccctcatcctgccgtcgaggaccgacgagaggccctgcgaaagacctctaccgacctgatggctgatcccgtggtgcgaatccaaaagaccttcttcattcccctgttcggcctgctgaacattatcctgcctatctggatcccctggtacttctggcaagagactctggtgcactgcttcgtggtgtctttcgtgacccgattcactatcaccctgaacattgctttctgcgtgaactctttcgcccacctgtggggcaacaagccctacgaccgattcatcaagcccgtcgagaaccagctcgtgtctctggccgctctcggcgaaggctggcataactaccaccacgtgttcccttgggactaccgaacctctgagctgggccgaatcaacatctctaccaacttcatcgacaccttcgccaagatcggctgggcctacgacctgaaggccgccacctctgccatgatcatccagcgagccaagcgatctggcgacggcaccctcggcgaggtcgaggaacccgatcctactctgtctaccaccgacaagccctctgtgtctcagggctag

SEQ ID NO: 53 - protein, 381 aa, Plodia interpunctella
MAPAQSNVIAQDQMHRQLKMRLIPRDQDCDKCDNTCDNNNSVLHNYSGSEKINSEAEFDLSKYEAIEFRPRIRWPDLTVQISLHLVAIYGLFLILTNQIKFYTLLFVLGTIYTSGFGITAGVHRLWSHRAYRARLPLRILLAFLFTITGQRDIYTWALDHRVHHKYTETVADPHDVRRGFWFAHVGWLVLTPHPAVEDRREALRKTSTDLMADPVVRIQKTFFIPLFGLLNIILPIWIPWYFWQETLVHCFVVSFVTRFTITLNIAFCVNSFAHLWGNKPYDRFIKPVENQLVSLAALGEGWHNYHHVFPWDYRTSELGRINISTNFIDTFAKIGWAYDLKAATSAMIIQRAKRSGDGTLGEVEEPDPTLSTTDKPSVSQG

SEQ ID NO: 54 - DNA, 1017 bp, Cadra cautella
  source 1..1017: mol_type = genomic DNA
atgacccctaaccctgagtctaccgccatcctggccgagggctctgaggaccctcgattcgagaagctggtggctccccaggctggaccccgaaagtacaagttcgtgctgctgaacatgatgtctcactcttactggcacctgtctgccatctacggcctgtacctgtgcttcacctctgccaagttcgccaccatgctgttcgccctgttcatcttcgtgatctctgacatcggcgtgaccgccggctctcaccgactgtgggctcaccgatcttacaaggtgaagctgcccctcgagatcctgctgatcatcatgaactctatcgcctaccaggacaccgccttcacctgggctcgagatcaccgactccaccaccgatacgccgacaccgacgctgaccctcacaactctacccgaggcttcttctactctcacatcggctggctgttcgtgcgaaagcaccacgaggtgatcgcccgaggcaagaccattcctatcgacgacctgaagaagaacaaggtcctgcagttccagcgaaagtacgcctacatcattatgtctaccctgtgtttcttcatgcccaccttcgtgcccgtgtacttctggggcgagtctatctctaccgcctggcacgtgaacctgctgcgatacgtgtgcaacctgaacgccatcttcctggtgaactctgcctctcacatgttcggctacaagccctacgacaagaacattgcctctgtgcagaacatccccgtgaccttcgtgaccctcggcgagggctaccacaactaccaccacacttacccctgggactacaaggccgccgagctgggcaacaacaagttcaacctgactaccaagttcatcgactttttcgcctggatcggctgggcctacgacctcaagaccgtgcctcaagagtctgtgtacaagcgaatggaacgaaccggcgacggcaccaacctgtggggctacgagcgagagaacatcgagaacgaggtgaactccgacttttag

SEQ ID NO: 55 - protein, 338 aa, Cadra cautella
MTPNPESTAILAEGSEDPRFEKLVAPQAGPRKYKFVLLNMMSHSYWHLSAIYGLYLCFTSAKFATMLFALFIFVISDIGVTAGSHRLWAHRSYKVKLPLEILLIIMNSIAYQDTAFTWARDHRLHHRYADTDADPHNSTRGFFYSHIGWLFVRKHHEVIARGKTIPIDDLKKNKVLQFQRKYAYIIMSTLCFFMPTFVPVYFWGESISTAWHVNLLRYVCNLNAIFLVNSASHMFGYKPYDKNIASVQNIPVTFVTLGEGYHNYHHTYPWDYKAAELGNNKFNLTTKFIDFFAWIGWAYDLKTVPQESVYKRMERTGDGTNLWGYERENIENEVNSDF

SEQ ID NO: 56 - DNA, 1077 bp, Drosophila yakuba
  source 1..1077: mol_type = genomic DNA
atggctccctacgagccctcttcgaactcttctaagcagaccggcgtgctgttcgagggcgacgccgagactgccgacggcgacctgacctctgaccgattccagctgaagcgagccgagaaccgacgactggacctggtgtggcgaaacatcatcctgttcgccctggtgcacctgtctgccctgtacggcctgtactctatcttcaccaaggccaagctggccaccactctgttcgctgccggactgtacatcatcggcatgctgggcgtgaccgctggcgcccaccgactgtgggctcaccgaacctacaaggctaagtggcccctgcgactgatcctggtgatcttcaacaccgtggccttccaggacgccgcctaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggcaagggcctcgacctgtccgacctgcgagctgaccccatcctgatgttccagcgaaagcactactacattctgatgcccctggcctgcttcgtgctgcccaccgtgattcccatggtgtactggaacgagactctggcctcttcctggttcgtggcctctatgttccgatggtgcttccagctcaacatgacctggctggtgaactctgccgctcaccagttcggcaaccgaccttacgacaagactatgaaccccactcagaacgcctacgtgtctgccttcaccttcggcgaaggctggcacaactaccaccactcgtttccctgggactacaagaccgccgagtggggccgatactctctgaacatcaccaccgccttcatcgacatgttcgccaagatcggctgggcctacgacctcaagaccgtggctcccgacatgatccagcgacgagtgctgcgaaccggcgacggctctcacgagctgtggggctggggcgacaaggacctgactcctgaggacgcccgaaacgtcctgttcgtggacaagcgatcttag

SEQ ID NO: 57 - protein, 358 aa, Drosophila yakuba
MAPYEPSSNSSKQTGVLFEGDAETADGDLTSDRFQLKRAENRRLDLVWRNIILFALVHLSALYGLYSIFTKAKLATTLFAAGLYIIGMLGVTAGAHRLWAHRTYKAKWPLRLILVIFNTVAFQDAAYHWARDHRVHHKYSETDADPHNATRGFFFSHVGWLLCKKHPDIKEKGKGLDLSDLRADPILMFQRKHYYILMPLACFVLPTVIPMVYWNETLASSWFVASMFRWCFQLNMTWLVNSAAHQFGNRPYDKTMNPTQNAYVSAFTFGEGWHNYHHSFPWDYKTAEWGRYSLNITTAFIDMFAKIGWAYDLKTVAPDMIQRRVLRTGDGSHELWGWGDKDLTPEDARNVLFVDKRS

SEQ ID NO: 58 - DNA, 1125 bp, Drosophila ananassae
  source 1..1125: mol_type = genomic DNA
atgcctccgaactctaaggaatctaccgacaagtgcaccggcctggcctctgaggtgcccggcaccgtggacgacggatcttctaagcagaccggcgtgctgttcgaggccgacgtcgagactaacgacggcgacctggccgtgtctaccgtcgagttcaagcgagccgagaagcgaaagctggaactggtgtggcgaaacatcatcctgttcgcctacgtgcacctggccgctctgtacggcggctacctgatgttcacccaggccaagctggccaccaccatcttcgccgctggcctgtacatctgcggcatgctgggcatcaccggcggagcccaccgactgtgggctcaccgatcttacaaggctaagtggcccctgcgactgatcctgatcgtgttcaacaccattgccttccaggacgccgcctaccactgggcccgagatcaccgagtgcaccacaagttctctgagactgacgctgaccctcacaacgctacccgaggcttcttcttctctcacgtcggctggctgctgaccaagaagcaccccgacgtcaaggccaagggcaagatcctggacctgtctgacctgcatgctgaccccattctgatgttccaaaagaagcactacttcgttctgatgcccctggcctgcttcattctgcccaccatcattcccgtgctgtgctggaacgagtctctgacctgtggctggctggtcgccaccatgttccgatggtgcttccagctgaacatgacctggctggtgaactctgccgctcacaagttcggcggacgaccctacgacaagaacatgaacccctctgagaacgcctacgtcgctaccatgaccttcggcgaaggctggcacaactaccaccacgtgttcccttgggactacaagacctctgagtggggcaaccgactcaacatgaccgctcgattcatcgacctgttcgctaagatcggctgggcctacgacctgaagtctgtggctcccgacaccgtgaagcgacgagtctggcgaaccggcgacggctctcacgagctgtggggctggggcgacaaggacctgactcctgaggacgccaaggacgtgctgtttgtggacaagatgaactag

SEQ ID NO: 59 - protein, 374 aa, Drosophila ananassae
MPPNSKESTDKCTGLASEVPGTVDDGSSKQTGVLFEADVETNDGDLAVSTVEFKRAEKRKLELVWRNIILFAYVHLAALYGGYLMFTQAKLATTIFAAGLYICGMLGITGGAHRLWAHRSYKAKWPLRLILIVFNTIAFQDAAYHWARDHRVHHKFSETDADPHNATRGFFFSHVGWLLTKKHPDVKAKGKILDLSDLHADPILMFQKKHYFVLMPLACFILPTIIPVLCWNESLTCGWLVATMFRWCFQLNMTWLVNSAAHKFGGRPYDKNMNPSENAYVATMTFGEGWHNYHHVFPWDYKTSEWGNRLNMTARFIDLFAKIGWAYDLKSVAPDTVKRRVWRTGDGSHELWGWGDKDLTPEDAKDVLFVDKMN

SEQ ID NO: 60 - DNA, 1116 bp, Choristoneura parallela
  source 1..1116: mol_type = genomic DNA
atggattttctgaacgagatcgacaattgccccgagcggctgagaaagccagagaagatggcccccaacgtgaccgaggagaatggcgtgctgttcgagtccgatgcagcaaccccagacctggccctggcaaggacacctgtggagcaggccgacgattctccaaggatctacgtgtggcgcaacatcatcctgtttgcctatctgcacctggccgccatctacggcggctatctgtttctgttctccgccaagtggcagaccgatatcttcgcctacctgctgtatgtggcatctctgctgggaatcacagcaggagcacacaggctgtgggcacacaagagctacaaggccaagtggcctctgcgcctgatcctgaccatctttaacacaatcgcctttcaggacagcgccatcgattgggccagggaccaccgcatgcaccacaagtattccgagaccgacgccgatccacacaatgccacacggggcttctttttctctcacatcggatggctgctggtgcggaagcacccagagctgaagagaaagggcaagggcctggacctgtctgatctgtacagcgatcccatcctgagatttcagaagaagtactatatgatcctgatgcctctggcctgtttcatcctgcccaccgtgatccccgtgtatatgtggaacgagacatggagcaatgcctttttcgtggccgccctgtttaggtataccttcatcctgaacgtgacatggctggtgaattccgccgcccacaagtggggcgataagccttacgacaagtccatcaagccatctgagaacatgagcgtgtccctgtttgccttcggcgagggctttcacaattaccaccacaccttcccttgggactataagacagccgagctgggcaaccaccggctgaacttcaccacaaagttcatcaacttcttcgccaagatcggctgggcctatgatatgaagaccgtgtctcaggagatcgtgcagcagcgggtgaagagaacaggcgacggaagccaccacctgtggggatggggcgacaaggatcacgcacaggaggagatcaacgccgccatccgcatcaatccaaaggacgattga

SEQ ID NO: 61 - protein, 371 aa, Choristoneura parallela
MDFLNEIDNCPERLRKPEKMAPNVTEENGVLFESDAATPDLALARTPVEQADDSPRIYVWRNIILFAYLHLAAIYGGYLFLFSAKWQTDIFAYLLYVASLLGITAGAHRLWAHKSYKAKWPLRLILTIFNTIAFQDSAIDWARDHRMHHKYSETDADPHNATRGFFFSHIGWLLVRKHPELKRKGKGLDLSDLYSDPILRFQKKYYMILMPLACFILPTVIPVYMWNETWSNAFFVAALFRYTFILNVTWLVNSAAHKWGDKPYDKSIKPSENMSVSLFAFGEGFHNYHHTFPWDYKTAELGNHRLNFTTKFINFFAKIGWAYDMKTVSQEIVQQRVKRTGDGSHHLWGWGDKDHAQEEINAAIRINPKDD

SEQ ID NO: 62 - DNA, 1389 bp, Ostrinia furnacalis
  source 1..1389: mol_type = genomic DNA
atgtctgccaacaccatggaaaccgacgagcagttcacctacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacgtggccgccaagctgatcttcaagatgtctctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctaccggcgtgcccctcgagactatctacgacaccctggacgccaagaacacccgactgatggacatcttcctggacaagcgacccaacacctacacctactccaaggctctggccgaggtggtcgtcgagaaggaattcgacgagtctgccgccatcgtgcgaccctctatcattgcctcttctattcgagagcccattcctggctggctgtctggctctcacggcttccctcgagtggtcgaggccgcctgcaagggcctgctgctccgatggcacggcgacggcaccgtggccttcgacgtgatccccgtggaccacgtcgccaacctgatcattgccgccgcttgggagtctaacgagcgacgactcatgggcaacaagggcgtgaaggtctacaactgctgctctggcctgcgaaaccctatcgacgtgtctaccgtgatgaacacctgtatcaagtaccgaaagtacttcggcacccgaaccatgtctatcatcacccctcgattcattatgaagaagaactacttcctgtacaagctgctgtacttcacctaccacactatccccgctgctattatcgacggcttcttctggctgaccggacgaacccctatgatgctgaacaccctgcacaagctccgaaagctgtcctctgtgctcgagtactttaccctgcaccagtttctgtttctggactccaacgtgcgaggactgctgcgacgaatggaatctaccgaccgacagaccttcaacttcgacgtcaccgagatcgagtgggagccctacctgcagaacttcgtgcgaggcattgccaacaactacaactattctatgtag

SEQ ID NO: 63 - protein, 462 aa, Ostrinia furnacalis
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLIFKMSLTAAVNINTKPTEQLIAICKKMRRNPIFIYVSSAYSNVNEQIIDEKVYSTGVPLETIYDTLDAKNTRLMDIFLDKRPNTYTYSKALAEVVVEKEFDESAAIVRPSIIASSIREPIPGWLSGSHGFPRVVEAACKGLLLRWHGDGTVAFDVIPVDHVANLIIAAAWESNERRLMGNKGVKVYNCCSGLRNPIDVSTVMNTCIKYRKYFGTRTMSIITPRFIMKKNYFLYKLLYFTYHTIPAAIIDGFFWLTGRTPMMLNTLHKLRKLSSVLEYFTLHQFLFLDSNVRGLLRRMESTDRQTFNFDVTEIEWEPYLQNFVRGIANNYNYSM

SEQ ID NO: 64 - DNA, 1392 bp, Ostrinia zag
  source 1..1392: mol_type = genomic DNA
atgtctgccaacaccgtcgaaaccgacgagcagttcaccgacaacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacctggccgccaagctgctcttcaagatgtctctgaccgccgctgtgaacatcaacaccaagcctaccgagcagctgatcgccatctgcaagaagatgcgacgaaaccccatcttcatctacgtgtcctctgcctactctaacgtgaacaagcagatcatcgacgagaaggtgtactctaccggcgtgcccctcgagactatctacgacaccctgaacaccgagaacacccgaatcaccgacatcctgctggacaagcgacccaacacctacacctactccaaggctctggccgaggtggtcgtcgagaaggaattcgacgagtctgccgccatcgtgcgaccctctatcattgtgtcttctattcgagagcccattcctggctggatgtctggctctcacggcttccctcgagtggtgggagccgcctgcgagggcctgctgctccgatggcacggcgactctaccgtggtgttcgacgtgatccccgtggaccacgtcgccaacctgatcattgccgccgcttgggagtctaacgagcgacgactcatgggcaacaagggcgtgaaggtctacaactgctgctctggcctgcgaaaccctatcgacgtgtctaccatcatgaacacctgtctgaagtaccgaaagtacttcggcacccgaaccctgtctatcttcacccctcgattcattgtgaagaagaactacttcatctacaagctgctgtacttcacctaccacactatccccgctgctattgtggacggcttcttctggctgacccgacgaacccctatgatgctgaacaccctgcacaagctctctaagctgtcctctgtgctcgagtactttaccctgcaccagtttctgtttctggactccaacgtgcgaggactgctgcgacgaatggaatctaccgaccgacagaccttcaacttcgacgtcaccgagatcgagtgggagccctacctgcagaactgtgtgcgaggaatcgccaacaactgcgactattctatgtag

SEQ ID NO: 65 - protein, 463 aa, Ostrinia zag
MSANTVETDEQFTDNNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHLAAKLLFKMSLTAAVNINTKPTEQLIAICKKMRRNPIFIYVSSAYSNVNKQIIDEKVYSTGVPLETIYDTLNTENTRITDILLDKRPNTYTYSKALAEVVVEKEFDESAAIVRPSIIVSSIREPIPGWMSGSHGFPRVVGAACEGLLLRWHGDSTVVFDVIPVDHVANLIIAAAWESNERRLMGNKGVKVYNCCSGLRNPIDVSTIMNTCLKYRKYFGTRTLSIFTPRFIVKKNYFIYKLLYFTYHTIPAAIVDGFFWLTRRTPMMLNTLHKLSKLSSVLEYFTLHQFLFLDSNVRGLLRRMESTDRQTFNFDVTEIEWEPYLQNCVRGIANNCDYSM

SEQ ID NO: 66 - DNA, 1389 bp, Ostrinia zea
  source 1..1389: mol_type = genomic DNA
atggccgccaacaccatcgaaaccgacgagcagttcaccgacaactctcccatcgtgaacttctactctggcaagtctgtgttcgtgaccggcgctaccggcttcgtgggcaccgtgctggtcgagaagctgctgttctcttgcaagggcatcaacaacatctacatcctgatcaagcagaccgaggacctgaccatcgaggcccgaatcctgaactacctgaactctaagcccttccaccgagtgaagaacactaaccccgagctgctgaagaagatcatccccatctgcggcaacctcgaggacaagaacctgggcatctctgactctgacatgaagaccctgctggaagaggtgtctatcgtgttccacgtggccgccaagctgctcttcaagatgaacctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctacccgagtgcccctcgagactgtgtacgacaccctggacatcgagaacacccgaattaccgacatcttcctggacaagcgacccaacacctacacctactccaaggctctggccgaggtggtcgtcgagaaggaattcaacgagtctgccgccatcgtgcgaccctctatcattctgtcttctattcgagagcccattcctggctggctgtctggctctcacggcttccctcgagtggtcggagccgcctgcaagggcctgctgctccgatggcacggcgacggcaccgtggtgtgcgacgtgatccccgtggaccacgtcgccaacctgtctattgccgtggcctgggagtctaacgagcgacgactcatgggcaacaagggcgtgaaggtctacaactgctgctcttctctgcgaaaccctatcgacgtgtctaccgtgctgaacacctgtctgaagtaccgaaagtacttcggcacccgaaccatgtctatcttcacccctcgattcattatgaagaagaactacttcctgtacaagctgctgtacttcacctaccacactatccccgctgctattatcgacggcttcttctggctgaccggacgaacccctatcatgctcaagaccctggacaagctccgaaagctgtcctctgtgctcgagtactttacccaccaccagtttctgtttctggactccaacgtgcgaggactgctgcgacgaatggaaggcaccgaccgacagaccttcaacttcgacgtcaccgagatcgagtgggagccctacctgcagaacttcgtgcgaggcattgccaacaactacgacaactctatgtag

SEQ ID NO: 67 - protein, 462 aa, Ostrinia zea
MAANTIETDEQFTDNSPIVNFYSGKSVFVTGATGFVGTVLVEKLLFSCKGINNIYILIKQTEDLTIEARILNYLNSKPFHRVKNTNPELLKKIIPICGNLEDKNLGISDSDMKTLLEEVSIVFHVAAKLLFKMNLTAAVNINTKPTEQLIAICKKMRRNPIFIYVSSAYSNVNEQIIDEKVYSTRVPLETVYDTLDIENTRITDIFLDKRPNTYTYSKALAEVVVEKEFNESAAIVRPSIILSSIREPIPGWLSGSHGFPRVVGAACKGLLLRWHGDGTVVCDVIPVDHVANLSIAVAWESNERRLMGNKGVKVYNCCSSLRNPIDVSTVLNTCLKYRKYFGTRTMSIFTPRFIMKKNYFLYKLLYFTYHTIPAAIIDGFFWLTGRTPIMLKTLDKLRKLSSVLEYFTHHQFLFLDSNVRGLLRRMEGTDRQTFNFDVTEIEWEPYLQNFVRGIANNYDNSM

SEQ ID NO: 68 - DNA, 1362 bp, Heliothis subflexa
  source 1..1362: mol_type = genomic DNA
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 69 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 70 - DNA, 1371 bp, Helicoverpa assulta
  source 1..1371: mol_type = genomic DNA
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 71 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 72 - DNA, 1350 bp, Yponomeuta rorellus
  source 1..1350: mol_type = genomic DNA
atggtccaattgaaagaagattccgttgctgctttttacgccgaaaagtctattttcattactggtggtactggtttcttgggtaaggttttgattgaaaagttgttgtactcctgcaaggccgttgatcaaatctacgttttgatcagaaagaagaaggaccaaaccccatctgaaagaattgcccaattattggaatccgaattattctccagattgagaaaggatgatccatccgctttgaaaaaggttgttccagttgttggtgatttgaccatgcctaatttgggtttgtctgctgctgttgaagatttgattgtctctaaggttaccgttatcttccatgttgctgctactgttaagttcaacgaaagaatgaagaacgccttggttaacaacgttgaagctactagagaagtcatcaacttgtgccatagattggaaaaggttgatgccttcattcatgtttctaccgcttactctaataccgatcaaaaggtcgttgaagaaagagtttatccaccaccagctccattgtctgaagtttatgcttttgttaagaactacggtgacgacatggatatcatccaaaatttgttgaacggtagaccaaacacttacacctacacaaaagctttggctgaagatatcgtcttgaaagaacatggtggtattccaaccgctattatcagaccatctatcgttttgtccgttttaaaagaacctatcccaggttggttggataattggaatggtccaactggtttgttgcatgcttcttcacaaggtgttcattgctctatgttgggttctggttctaatgttgccgatttgattccagttgatatcgtcactaacttgatgatcgttgttgcttctagatgcagaaagtctaatggtttgaaggtctacaactcttgttctggtactaccaacccaattacttaccaagctttcaccaagatgttcttggattcctgtatttctagaggttggaacaaggttccattccctttgttgatttttgtcaagtgggctttcttgaacagagtcttgaagttcttgttggttatcgtcccattcttcttgatcgatgtctacttgagatttttcggtaagccaaactacatgagaatgatcacctacaccaagaaagctgaagatttgatgaccttcttcacctctcatgaatggcaattcaaggatggtaatgtcagagacttgatcaacatgatgtctccagaagatagaaagatcttctactgcgatcctgaagaaattcaatggaagccatacttcgatgattactgcgttggtgtctttaaatacttgttgaagagaaaggtttaa

SEQ ID NO: 73 - protein, 449 aa, Yponomeuta rorellus
MVQLKEDSVAAFYAEKSIFITGGTGFLGKVLIEKLLYSCKAVDQIYVLIRKKKDQTPSERIAQLLESELFSRLRKDDPSALKKVVPVVGDLTMPNLGLSAAVEDLIVSKVTVIFHVAATVKFNERMKNALVNNVEATREVINLCHRLEKVDAFIHVSTAYSNTDQKVVEERVYPPPAPLSEVYAFVKNYGDDMDIIQNLLNGRPNTYTYTKALAEDIVLKEHGGIPTAIIRPSIVLSVLKEPIPGWLDNWNGPTGLLHASSQGVHCSMLGSGSNVADLIPVDIVTNLMIVVASRCRKSNGLKVYNSCSGTTNPITYQAFTKMFLDSCISRGWNKVPFPLLIFVKWAFLNRVLKFLLVIVPFFLIDVYLRFFGKPNYMRMITYTKKAEDLMTFFTSHEWQFKDGNVRDLINMMSPEDRKIFYCDPEEIQWKPYFDDYCVGVFKYLLKRKV

SEQ ID NO: 74 - DNA, 1377 bp, Agrotis segetum
  source 1..1377: mol_type = genomic DNA
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 75 - protein, 458 aa, Agrotis segetum
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 76 - DNA, 1437 bp, Bicyclus anynana
  source 1..1437: mol_type = genomic DNA
atgtaccgaaacgtgaacaacaactacaagggccactctgtgtacaccaacggcgctggctctcgagtgaagtctctgctgtcctctgccaccgacaacaccaacgagtaccagtctatcgccgagtgctacaagggacagtccgtgttcatcaccggcggcaccggcttcgtcggcaaggtgctgctcgagaagctgctgtactcttgccccggcatcgacaaggtgtacctgctggtgcgagagactcagggcgccaccgctcaccagcgaatgcagaagctcctggaagaacccgccttctcgcgaatcaaggaagagaaccctcaggccttcgagaaggtgatccccatcgtgggcgacatcacccagcctcagctgggcatcatggccgagaacgaggaactgctgattaaggaagtgtctttcgtctaccacgtggccgccaccaccaagttcaacgagactctggacattgccatgaacgtgaacgtggccggaaccggacgagtgctggacctgtctaagcgaatggaaaacatcaaggccttcgtgtacgtgtctaccgcctactctaacaccgaccgagaggtggtggaagaggtgctgtaccccgctcctgtgtctctgaacgaggtgcacaagctgctgaagatcggcatcaccgacgctcaggtgaaggaactgatcaagggacgacccaacacctacaccttcaccaaggctctggctgagaacctggtggccgacaaccacggacacgtgcccgccatcatcgtgcgaccctctatcgtgtcctcgtctaagaaggaacccatcaccggatggatcgactcttggtacggcgccaccttcctggccaccgtgaccatgaagggcttcaaccgagtgttcgtgtcgtcttacgagtacaacctggacttcatccccgtggactacgtgtccaacctgatcatcgtggccgctgctcgatgcaagtgctctgacaaggtggacgtgtacaactcttctacctccggcgaaaaccctctgaagattggcgccttcttcgacgacatcattgcctactcttgcaagcacaagttctacgacatccctctgcctatggcctacctgactcgataccgatgggtcatgttcctgatcaccctgctgctgcagaccctgcctgcctatatcgccgacctgttcctgctgatcgtgggcaagaagccccgatacgtcaagctggcctctaagatctctgccgctcacgaggtcctggactacttcccctctcgaacctggtctatgtctgcccgacagaccaccgctctgttccagtctctgtctccctcggaccgagatcagtttccttgcgaccccaccgacatcgactggaaggagtacatcgtcacctactgccagggaatccgacagttcctgtgcaagtcttaa

SEQ ID NO: 77 - protein, 478 aa, Bicyclus anynana
MYRNVNNNYKGHSVYTNGAGSRVKSLLSSATDNTNEYQSIAECYKGQSVFITGGTGFVGKVLLEKLLYSCPGIDKVYLLVRETQGATAHQRMQKLLEEPAFSRIKEENPQAFEKVIPIVGDITQPQLGIMAENEELLIKEVSFVYHVAATTKFNETLDIAMNVNVAGTGRVLDLSKRMENIKAFVYVSTAYSNTDREVVEEVLYPAPVSLNEVHKLLKIGITDAQVKELIKGRPNTYTFTKALAENLVADNHGHVPAIIVRPSIVSSSKKEPITGWIDSWYGATFLATVTMKGFNRVFVSSYEYNLDFIPVDYVSNLIIVAAARCKCSDKVDVYNSSTSGENPLKIGAFFDDIIAYSCKHKFYDIPLPMAYLTRYRWVMFLITLLLQTLPAYIADLFLLIVGKKPRYVKLASKISAAHEVLDYFPSRTWSMSARQTTALFQSLSPSDRDQFPCDPTDIDWKEYIVTYCQGIRQFLCKS

SEQ ID NO: 78 - DNA, 1362 bp, Heliothis virescens
  source 1..1362: mol_type = genomic DNA
atggttgtcttgacctccaaagaaactaagccatctgttgctgaattttacgctggtaagtctgttttcattactggtggtactggtttcttgggtaaggttttcattgaaaagttgttgtactcctgcccagatatcgttaacatctacatgttgatcagagaaaagaagggtttgtccgtttccgaaagaatcaagcaatttttggatgaccctttgttcaccagattgaaggacaaaagaccagctgatttggaaaagatcgttttgattccaggtgatattaccgctccagatttgggtattactgctgctaacgaaaagatgttgatcgaaaaggtttccgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagtttaccaatacgtcaaagaaggtatctccgaagaagataccgaaaaaatcttgaacggtagaccaaacacttacactttcactaaggctttgaccgaacatttggttgctgaaaatcaagcttacgttccaaccattatcgttagaccatcagttgttgctgccattaaggatgaaccattgaaaggttggttgggtaattggtttggtgctacaggtttgactgtttttactgctaagggtttgaacagagttatctacggtcattccaactacatcgttgatttgatcccagttgattacgttgccaacttggttattgctgctggtgctaaatctaacacctcttctgaattgaaggtctacaactgttgttcctcatcatgtaacccagttaagatcggtactttgatgtctatgtttgctgatgatgccatcaagcaaaagtcttatgctatgccattgccaggttggtacatttttactaagtacaagtggttggtcttgttgttgaccttcttgttccaagttattccagcctacattaccgatttgtcaagacacttggttggtaagagtccaagatatatcaagttgcaatccttggtcaatcaaaccagatcctccattgatttcttcaccaatcattcttgggttatgaaggccgatagagtcagagaattatacgcttctttgtctccagcagataagtacttgtttccatgtgatccagttaacatcaactggacccaatacttgcaagattactgttggggtgttagaaacttcttggaaaaaaagacttaa

SEQ ID NO: 79 - protein, 453 aa, Heliothis virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 80 - protein, 344 aa, synthetic construct
MVPNKGSSVVLTENAEPQFKKLVAPQAGPRKYQFVYLNMMTHGYWHLSAIYGLYLSFTTAKWATLIFALFIFVISKISVTAGSHRLWAHRSYKAKLPLEILLIIMNSIAFQDTAFTWARDHRLHHRHADTDADPHNSTRGFFYSHIGWLFVKKHPEVVARGKTIPIEDLKKNKLLQFQRKYAFPLMGTLCFFMPTFVPVYFWGESVITAWHINLLRYVCNLNAIFSVNSAAHMFGYKPYDKKIASVQNLPVTFVTLGEGYHNYHHTYPWDYRAAELGNNKYNLTTKFIDFFAWIGWAYDLKTVPQEAVEKRMARTGDGTDLWGYGEKKPVKDVKDNSDETYYDF

SEQ ID NO: 81 - protein, 344 aa, synthetic construct
MVPNKGSSVVLTENAEPQFKKLVAPQAGPRKYKFVLLNMMSHSYWHLSAIYGLYLCFTSAKFATMLFALFIFVISDIGVTAGSHRLWAHRSYKVKLPLEILLIIMNSIAYQDTAFTWARDHRLHHRYADTDADPHNSTRGFFYSHIGWLFVRKHHEVIARGKTIPIDDLKKNKVLQFQRKYAYIIMSTLCFFMPTFVPVYFWGESISTAWHVNLLRYVCNLNAIFLVNSASHMFGYKPYDKNIASVQNIPVTFVTLGEGYHNYHHTYPWDYKAAELGNNKFNLTTKFIDFFAWIGWAYDLKTVPQEAVEKRMARTGDGTDLWGYGEKKPVKDVKDNSDETYYDF

SEQ ID NO: 82 - protein, 326 aa, synthetic construct
MVPNKGSSDVLSEHSEPQFTKLVAPQAGPRKYKFVLLNMMSHSYWHLSAIYGLYLCFTSAKFATMLFALFIFVISDIGVTAGSHRLWAHRSYKVKLPLEILLIIMNSIAYQDTAFTWARDHRLHHRYADTDADPHNSTRGFFYSHIGWLFVRKHHEVIARGKTIPIDDLKKNKVLQFQRKYAYIIMSTLCFFMPTFVPVYFWGESISTAWHVNLLRYVCNLNAIFLVNSASHMFGYKPYDKNIASVQNIPVTFVTLGEGYHNYHHTYPWDYKAAELGNNKFNLTTKFIDFFAWIGWAYDLKSVPQEAIAKRCAKTGDGTDMWGRKR

SEQ ID NO: 83 - protein, 326 aa, Amyelois transitella
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 84 - DNA, 1041 bp, Ephestia kuehniella
  source 1..1041: mol_type = genomic DNA
atgacccctaaccctggctctaccgccatgctcgaggactctgagcctcctcagttcgagaagctggtggctccccaggctggaccccgaaagtacgagttcgtgtacctgaacatgatgacccacgcctactggcacctgtcctctatctactctctgtacctgtgcttcacctctgccaagtgggctaccctgctgttcgccctgttcatcttcatcatctctgacatctctgtgaccggcggctctcaccgactgtgggctcaccgatcttacaaggccaagctgcccctcgagatcctgctgatcatcatgaactgtatcgccttccaggacaccgcctacacctgggctcgagatcaccgactccaccaccgatactctgacaccgacgctgaccctcacaacgctacccgaggcttcttctactctcacatcggctggctgttcgtgcgaaagcaccccgaggtggtggcccgaggcaagaccatccagatcgacgacctgaagaagaacaagctgctgcagttccagagaaagtacgctttccctctgatgggcaccttctgtttcttcatgcccaccttcgtgcccgtgtacttctggggcgagtctatcaacaccgcctggcacatcaacctgctgcgatacgtgtgcaacctgaacgccatcttcctggtgaactctgccgctcacatgttcggctacaagccctacgacaagaacattgcctctgtgcagaacctggccgtgaccgtggctaccctcggcgagggctaccacaactaccaccacacttacccctgggactaccgagccgccgagctgggcaacaacaagttcaacctgactaccaagttcatcgactttttcgcctgggtcggctgggcctacgacctcaagaccgtgcctcaagagtctatccgaaagcgaatggcccgaaccggcgacggcaccgacctctggggctacgagcgaaagaacctggagtacgacgacgaggccaacaacgacttcatcaagaacgacatcgtgcactaa

SEQ ID NO: 85 - protein, 346 aa, Ephestia kuehniella
MTPNPGSTAMLEDSEPPQFEKLVAPQAGPRKYEFVYLNMMTHAYWHLSSIYSLYLCFTSAKWATLLFALFIFIISDISVTGGSHRLWAHRSYKAKLPLEILLIIMNCIAFQDTAYTWARDHRLHHRYSDTDADPHNATRGFFYSHIGWLFVRKHPEVVARGKTIQIDDLKKNKLLQFQRKYAFPLMGTFCFFMPTFVPVYFWGESINTAWHINLLRYVCNLNAIFLVNSAAHMFGYKPYDKNIASVQNLAVTVATLGEGYHNYHHTYPWDYRAAELGNNKFNLTTKFIDFFAWVGWAYDLKTVPQESIRKRMARTGDGTDLWGYERKNLEYDDEANNDFIKNDIVH

SEQ ID NO: 86 - DNA, 1038 bp, Ephestia elutella
  source 1..1038: mol_type = genomic DNA
atgacccctaaccctgagtctatcgccatcctgtctgaggactacgagccctctcagtacgagaagctggtggctccccaggctggaccccgaaagtacaagttcgtgtacctgaacatgatgacccactcttactggcacctgtctgccatctacggcctgtacctgtgcttcacctctgccaagctggctaccctgatcttcgccctggtgctgctgcgaatctcttctctgggcgtgaccggcggctctcaccgactgtgggctcaccgatcttacaaggtgaagctgcccctcgagatcctgctgatcatcatgaactctatcgccttccaggacaccgccttcacctgggctcgagatcaccgactccaccaccgatacgccgacaccgacgctgaccctcacaactctacccgaggcttcttctactctcacatcggctggctgttcgtgaagaagcaccctcaggtcgtcgcccgaggcaagaccattcctatcgacgacctcaagaagaacaagctgctgcagttccagcgaaagtacgctttccccattatcggcaccctgtgtttcttcatgcccaccttcgtgcccgtgtacttctggggcgagaacatcatcaccgcctggcacatcaacctgctgcgatacgtgtgcaacctgaacgccatcttcctggtgaactctgcctctcacatgttcggctacaagccctacgacaagtctattgcctctgtgcagaacatcaccgtgtctctggtgaccctcggcgagggctaccacaactaccaccacacttacccctgggactaccgagccgccgagctgggcaacaaccgattcaacctgactaccaagttcatcgactttttcgcctggatcggctgggcctacgaccttaagaccgtgcctcaagagactatccaaaagcgaatgacccgaaccggcgacggcaccgacctctggggctacgagcgagagcgactcgagtacgagggaaacaacgacctgatcaagaacgacttctaccactag

SEQ ID NO: 87 - protein, 345 aa, Ephestia elutella
MTPNPESIAILSEDYEPSQYEKLVAPQAGPRKYKFVYLNMMTHSYWHLSAIYGLYLCFTSAKLATLIFALVLLRISSLGVTGGSHRLWAHRSYKVKLPLEILLIIMNSIAFQDTAFTWARDHRLHHRYADTDADPHNSTRGFFYSHIGWLFVKKHPQVVARGKTIPIDDLKKNKLLQFQRKYAFPIIGTLCFFMPTFVPVYFWGENIITAWHINLLRYVCNLNAIFLVNSASHMFGYKPYDKSIASVQNITVSLVTLGEGYHNYHHTYPWDYRAAELGNNRFNLTTKFIDFFAWIGWAYDLKTVPQETIQKRMTRTGDGTDLWGYERERLEYEGNNDLIKNDFYH

SEQ ID NO: 88 - protein, 669 aa, Agrotis segetum
MNTMDGSPGRENQKSTRKISLPTVLCTPEPSDDRSDMKVNSFMSSDSKMSLPTNLGTPTSPLRLSSPISLPTSPCVKTSQTSSANSLPNPNPKPVTSSAATNNIVNSGSATGNPRNKCALQPGHSLMDWIRLGNSGKDLTGVGGRIRPVSPAELSTHNTLNDAWLAIRGRVYNITHYLPYHPGGPEELMRGAGMDATQLFDKVHPWVNYDSLLAKCLVGPLRFELPDAEELFDTSNPSPKSDRLREPSKAQELVRKSMENLANCITPVRKKITKGDDNTKGSPPSKIMQSLVQSADLPMSISRRAASSPVKKTEKSPDSPLPLRYDWIQTSTKLTIYVYTGHLANPGGCARITEGFLLIEVATNGWLRTLKIKPEAKLKEPLQLRVFAESGKIEITALKVDPSVWKGCGDVSVGVASHVSSPRSVECRVMEVSRVSHDTSLLSVCPRAGPVVVPLGHHVRVHRRIEGTECIRSYTPVGEGWGPADGTEVFSALRLAVKRYDSGALSPHLTALKVGDLITLSGPYGNFQLQKLKTVKSLYLIAAGTGVTPMLGLIRFMISRSNPRCERTHLLFFNKTEEDILFRENFEEMMKEDDRLKITHVLSEGSSSWSGHRGRIRDELLAETIGNVKCDDKCSHYACLCGPTEFTYAGLDLLKKHGFKDDCIHAFMG

SEQ ID NO: 89 - protein, 578 aa, Bombus terrestris
MKKSLSKNQRKSDDTETKDIRGKMEEIDVKSSSSTNSMTNNLALLPSGVGRMKQLQGAKMVNQTGSSSSGSATGNPRNKTALAPGHSLMDWIRLGNSGVDLTGVGGVPRVVTLSELATHNKQDDAWIAIRGIVFNVTRYMDFHPGGVNELMRGVGKDATKLFENVHAWVNYQSILQKCVVGRLSRGSVSGTTSSPTENPVSSTSNCSSATLNLITSCTTHSVSQENVSDASSLNIKMDWRQTSDSLTLSYQTTRYYPGLCYQLSRINDSKLMYKLVFASGSITHELELTAEIQWPPVCIKNFETREITFTFMKQKQELWKSYGEQMVCREMNRDNRTYREYQVLTNKQLSKLVHLLVVRAKDFLQIVPIGRHVEAMLNVMGTEVSRLYTPVPPCLHPDDMAPNYKSDCLCFMIKKYPNGALSPSITALQIGETLRLSNALGDFTVESCDGYSINHMIAGGTGLTAMLGIIQRALARRSVKTINLLNFNKDEDNMFYVKELEKASADNKLKVTHILSQAGDAWTGRRGTLSDDLLQDLVGTSNPDAFVYTCGPQEFIQLAKNSLQKLNWKPFQMYEFDD

SEQ ID NO: 90 - protein, 678 aa, Lobesia botrana
MADPPAEDNTPSRNGSLKISIPTVLCTPESSENSDILCKTKSLLSSESKLSLPSNLGTPTSPLRLSSPLSLPASPTPFMKVTQPPPEKVTSGQMNSPPSTIVLQSGPKPPLANTGSATGNPRNKCALQPGHSLMDWIRLGNSGKDLTGVGGRMRPVSPDELASHNSQNDAWLAIRGRVYNITHYLPYHPGGPEELMRGAGIDATQLFDKVHPWVNYDSLLAKCLVGPLRLERPDAEELFGPSTPSPKTENRLREPSKAQELVRKSMENLANCITPVRKKISAKSEESVKGSPPSKIMQSLIQSSDLPVSISRRAAANPVKTSEKTDVAPSPLRFDWIQTSTKLTLSIYTGPLVNPGVCARIIEGILFVEVATNGWVRTLKLVPEAKLQDSLQLRVFAESGKIEVVAQKVEPGVWKGCGDAVSGAASHVPTPRSVVCRVSRVTRVSHDTVLLSLATPAQALVAPLGHHVRVHRTISDVECVRSYTPVGEGWTSDGERDSAIHLAVKGYETGALSPHLVALQPDDEVTVSGPYGNFELQKLRGVKEICFVAAGTGITPMLGLLKFILPRSNPRCERVHLLFFNKTEEDILFRQNFEEIAKEDDRFSVTHVLSDASSSWSGRRGRITSDLLTQVVGKDSLKCREQCVHFACVCGPTDFTHTTIDLLKKLHMKDSCIHAFMG

SEQ ID NO: 91 - RNA, 23 nt, synthetic construct
  source 1..23: mol_type = other RNA
actttttgcagtactaaccgcag

(19) United States
(12) Patent Application Publication
(10) Pub. No.: US 2024/0279693 A1
(43) Pub. Date: Aug. 22, 2024

(54) METHODS AND YEAST CELLS FOR PRODUCTION OF DESATURATED COMPOUNDS

(71) Applicants: FMC Agricultural Solutions A/S, Harboøre (DK); BIOPHERO APS, Copenhagen Ø (DK)

(72) Inventors: Carina Holkenbrink, Harboøre (DK); Irina Borodina, Harboøre (DK)

(21) Appl. No.: 18/570,268

(22) PCT Filed: Jul. 1, 2022
(86) PCT No.: PCT/EP2022/068272

§ 371 Date: Dec. 14, 2023

(30) Foreign Application Priority Data
Jul. 2, 2021 (EP) 21183447.8
Mar. 9, 2022 (EP) 22161107.2

(51) Int. Cl. C12P 7/6427; C12N 9/02

(52) U.S. Cl. CPC C12P 7/6427; C12N 9/0008; C12N 9/0071; C12Y 102/0105; C12Y 114/19; C12Y 114/19006

(57) ABSTRACT

The present invention relates to yeast cells capable of producing Δ(12) desaturated fatty acyl-CoAs and optionally fatty alcohols, said yeast cells expressing heterologous Δ(12) desaturases capable of introducing a double bond at position (12), i. e. a double bond between the carbon at position (12) and the carbon at position (13), in a saturated or desaturated fatty acyl-CoA having a carbon chain length of at least (13).

### TECHNICAL FIELD

The present invention relates to yeast cells capable of producing Δ12 desaturated fatty acyl-CoAs and optionally desaturated fatty alcohols, said yeast cells expressing heterologous Δ12 desaturases. Said compounds are precursors of sex pheromone components of several insects, such as Plodia interpunctella, Spodoptera exigua, Cadra cautella, Spodoptera eridania, and others.

### BACKGROUND

Integrated Pest Management (IPM) is expected to play a major role for both increasing the crop yield and for minimizing environmental impact and enabling organic food production. IPM employs alternative pest control methods, such as mating disruption using pheromones, mass trapping using pheromones, beneficial insects, etc.

Pheromones constitute a group of diverse chemicals that insects (like other organisms) use to communicate with individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides. The biological production of pheromones for use pest control is advantageous over chemical synthesis in respect to price, specificity, and environmental impact.

Pheromones of interest are (Z9, E12)-tetradecadien-1-ol, (Z9, E12)-tetradecadienal and (Z9, E12)-tetradecadienyl acetate. These compounds are either alone or in a mixture pheromones of a number of Lepidoptera moths, such as Plodia interpunctella, Spodoptera exigua, Cadra cautella, Spodoptera eridania and others. These moths are pests of significant economic importance in storage and horticultural crops.

Xia et al. (2019) discloses a yeast cell expressing a desaturase from Spodoptera exigua named SexiDes5. The major product of the SexiDes5 desaturase is (Z11)-hexadecanoic acid, while it was also found to produce small amounts of (Z9, E12)-tetradecanoic acid besides other mono- and double-desaturated fatty acids.

There remains a need for microbial cell factories which can produce Δ12 desaturated compounds with high titers.

### SUMMARY

The invention is as defined in the claims.

The present disclosure provides yeast cells capable of producing Δ12 desaturated fatty acyl-CoAs and optionally Δ12 desaturated fatty alcohols, said yeast cells expressing a heterologous Δ12 desaturase. In particular, the present disclosure provides yeast cells capable of producing (Z9, E12)-tetradecadienoic acid or (Z9, E12)-tetradecadien-1-ol, the precursors of the pheromones (Z9, E12)-tetradecadienal and (Z9, E12)-tetradecadien-1-ol acetate. (Z9, E12)-tetradecadien-1-ol, (Z9, E12)-tetradecadienal and (Z9, E12)-tetradecadien-1-ol acetate are sex pheromone components of Plodia interpunctella. Further, (Z9, E12)-tetradecadien-1-ol and/or (Z9, E12)-tetradecadien-1-ol acetate are pheromone components of Spodoptera exigua, Cadra cautella, Spodoptera eridania, and others. Cadra cautella is also known and referred to as Ephestia cautella. (Z9, E12)-tetradecadien-1-ol acetate is also known and referred to as (Z9, E12)-tetradecadien-1-yl acetate.

Thus, provided herein is a yeast cell capable of producing a desaturated fatty acyl-coenzyme A (fatty acyl-CoA) having n′ double bond(s), wherein at least one of said double bond(s) is at position 12,
wherein said yeast cell expresses a heterologous Δ12 fatty acyl-CoA desaturase, said desaturase being capable of introducing a double bond at position 12 in a saturated or desaturated fatty acyl-CoA, preferably a desaturated fatty acyl-CoA, having a carbon chain length of at least 13 and having n double bond(s),
wherein n and n′ are integers,
wherein 0≤n≤3 and wherein 1≤n′≤4.

Also provided herein is a method for producing a desaturated fatty acyl-CoA having n′ double bond(s), wherein at least one of said double bond(s) is at position 12, in a yeast cell, said method comprising the steps of:
i) providing a yeast cell as disclosed herein,
ii) incubating said yeast cell in a medium under conditions allowing expression of a heterologous Δ12 desaturase, said desaturase being capable of introducing a double bond at position 12 in a saturated or desaturated fatty acyl-CoA, preferably a desaturated fatty-acyl-CoA, having a carbon chain length of at least 13 and having n double bonds,
   wherein n and n′ are integers,
   wherein 0≤n≤3 and wherein 1≤n′≤4,
   thereby producing a desaturated fatty acyl-CoA having n′ double bond(s), wherein at least one of said double bond(s) is at position 12.

Further provided herein is a pheromone composition obtainable by a method comprising the following steps:
i) producing (Z9, E12)-tetradecadien-1-ol and/or an E12-fatty alcohol by the method as disclosed herein;
ii) recovering the (Z9, E12)-tetradecadien-1-ol and/or E12-fatty alcohol;
iii) converting the E12-fatty alcohol and/or (Z9, E12)-tetradecadien-1-ol into an E12-fatty alcohol acetate and/or (Z9, E12)-tetradecadien-1-ol acetate, respectively;
iv) converting the E12-fatty alcohol and/or (Z9, E12)-tetradecadien-1-ol into an E12-fatty aldehyde and/or (Z9, E12)-tetradecadienal, respectively;
v) formulating said E12-fatty alcohol, (Z9, E12)-tetradecadien-1-ol, E12-fatty alcohol acetate, (Z9, E12)-tetradecadien-1-ol acetate, E12-fatty aldehyde, and/or (Z9, E12)-tetradecadienal as a pheromone composition.

Further provided herein is a pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein the pheromone composition comprises at least 20% biobased carbon, such as at least 30%, such as at least 40%, such as at least 50%, such as at least 60%, such as at least 70%, such as at least 80%, such as at least 85%, such as at least 90%, such as at least 95% biobased carbon.

Also provided herein is a pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein the pheromone compound comprises at least 20% biobased carbon, such as at least 30%, such as at least 40%, such as at least 50%, such as at least 60%, such as at least 70%, such as at least 80%, such as at least 85%, such as at least 90%, such as at least 95% biobased carbon.

Further provided herein is a pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein the pheromone composition has a radioactive ¹⁴C level of at least 20%, such as at least 30%, such as at least 40%, such as at least 50%, such as at least 60%, such as at least 70%, such as at least 80%, such as at least 85%, such as at least 90%, such as at least 95%.

Also provided herein is a pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein the pheromone compound has a radioactive ¹⁴C level of at least 20%, such as at least 30%, such as at least 40%, such as at least 50%, such as at least 60%, such as at least 70%, such as at least 80%, such as at least 85%, such as at least 90%, such as at least 95%.

Also provided herein is the use of a Plodia interpunctella desaturase in a method for introducing a double bond at position 12 in a saturated or desaturated fatty acyl-CoA, optionally wherein the Plodia interpunctella desaturase is Pid12 (SEQ ID NO: 2) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

Also provided herein is the use of a Cadra cautella desaturase in a method for introducing a double bond at position 12 in a saturated or desaturated fatty acyl-CoA, optionally wherein the Cadra cautella desaturase is EcauDes12 (SEQ ID NO: 55) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

Further provided herein is the use of an Ephestia kuehniella desaturase in a method for introducing a double bond at position 12 in a saturated or desaturated fatty acyl-CoA, optionally wherein the Ephestia kuehniella desaturase is Eku_d12 (SEQ ID NO: 85) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

### DESCRIPTION OF DRAWINGS

FIG. 1. Pathway for production of (Z9, E12)-tetradecadien-1-ol (Z9, E12-14:OH) in yeast and enzymatic or chemical conversion to (Z9, E12)-tetradecadien-1-ol acetate (Z9, E12-14:OAc) or (Z9, E12)-tetradecadienal (Z9, E12-14:Ald). Abbreviations: 14:CoA, tetradecanoyl-CoA; Z9-14:CoA, (Z9)-tetradecenoyl-CoA; Z9,E12-14:CoA, (Z9, E12)-tetradecadienoyl-CoA.

FIG. 2. GC chromatograms of (A) Z9, E12-14:OH analytical standard, (B) extracts of control strain ST10628, (C) extracts of strain ST10870 expressing Pid12 and the mass spectra of the (D) analytical standard and (E) extract of ST10870 at retention time 10.2 min. Arbitrary units.

FIG. 3. (A) GC chromatograms of starting material A and product B of Example 12 and (B) mass spectra of starting material A and product B of Example 12.

FIG. 4. (A) GC chromatograms of extracts of control strain ST10748, extracts of strain ST12596 expressing Eku_d12, and Z9, E12-14:Me analytical standard, and the mass spectra of (B) the extract of ST12596 and (C) the analytical standard of Z9,E12-14:Me at retention time 10.39 min. Arbitrary units.

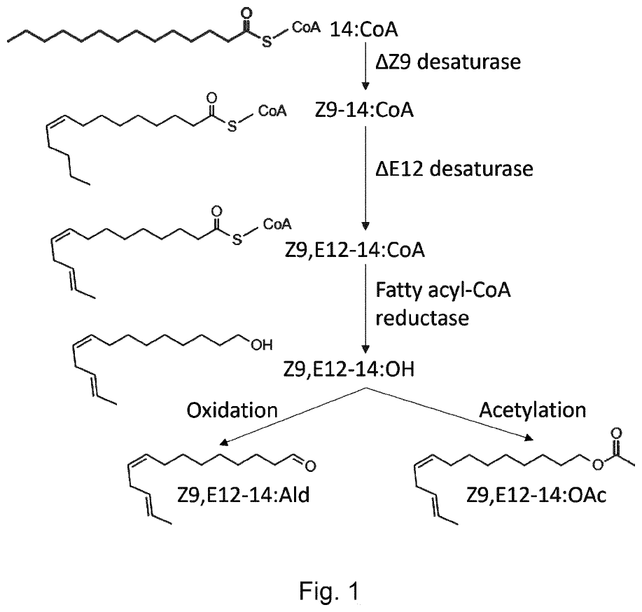

### DETAILED DESCRIPTION

### Definitions

Heterologous Δ12 desaturase: the term “heterologous Δ12 desaturase” as used herein refers to a desaturase, which is different from “further heterologous desaturase”. The heterologous desaturase is capable of introducing a double bond at position 12 in a saturated or desaturated fatty acyl-coenzyme A (fatty acyl-CoA).

A further heterologous desaturase: the term “a further heterologous desaturase” as used in herein refers to a desaturase which is different from “a heterologous desaturase”. The further heterologous desaturase is capable of introducing a double bond at any other position than position 12 in a saturated or desaturated fatty acyl-CoA. The further heterologous desaturase may be native to the same organism as the heterologous Δ12 desaturase, or it may be native to a different organism than the heterologous Δ12 desaturase, and catalyses a different reaction than the heterologous Δ12 desaturase.

Biopesticide: the term “biopesticide” is a contraction of ‘biological pesticide’ and refers to several types of pest management intervention: through predatory, parasitic, or chemical relationships. In the EU, biopesticides have been defined as “a form of pesticide based on micro-organisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present invention relates more particularly to biopesticides comprising natural products or naturally occurring substances. In the present context, these are manufactured by cultivating and concentrating naturally occurring organisms and/or their metabolites including bacteria and other microbes, fungi, nematodes, proteins, etc. These compounds are considered to be important components of integrated pest management (IPM) programmes, and have received much practical attention as substitutes to synthetic chemical plant protection products (PPPs). The Manual of Biocontrol Agents (2009: formerly the Biopesticide Manual) gives a review of the available biological insecticide (and other biology-based control) products.

Desaturated: the term “desaturated” will be herein used interchangeably with the term “unsaturated” and refers to a compound containing one or more double or triple carbon-carbon bonds, preferably a double carbon-carbon bond. The following nomenclature is used herein throughout: a Δi desaturated compound, where i is an integer, refers to a compound having a double or triple carbon-carbon bond between the carbon atom at position i of the carbon chain, and the carbon atom at position i+1 of the carbon chain. The carbon chain length is thus at least equal to i+1. For example, a Δ12 desaturated compound refers to a compound having a double or triple carbon-carbon bond between carbon 12 and carbon 13, and is herein referred to as a carbon chain having a carbon-carbon bond at position 12. Said Δ12 desaturated compound can have a carbon chain length of 13 or more. The double or triple bond can be in an E configuration or in a Z configuration. Thus, herein, an Ei or a Zi desaturated compound will refer to a compound having a double carbon-carbon bond in an E configuration or in a Z configuration, respectively, between carbon i and carbon i+1 of the carbon chain, wherein said desaturated compound has a total length at least equal to i+1. For example, an E12 desaturated fatty alcohol has a desaturation at position 12 (i.e. a double bond between carbon atom 12 and carbon atom 13) in an E configuration, and has a carbon chain length of 13 or more.

Derived from: the term “derived from” when referring to a polypeptide or a polynucleotide derived from an organism means that said polypeptide or polynucleotide is native to said organism, i.e. that it is naturally found in said organism.

E12-fatty acyl-CoA having at least one double bond at position 12: the term “E12-fatty acyl-CoA having at least one double bond at position 12” as used herein, means that the E12-fatty acyl-CoA has one double bond at position 12. In some embodiments of the invention, the E12-fatty acyl-CoA only has the double bond at position 12. In other embodiments of the invention, the E12-fatty acyl-CoA has additional double bonds beside the double bond at position 12. The additional double bonds can be at any position other than position 12. One example hereof is an E12-fatty acyl-CoA having a double bond at position 12 and an additional double bond at any other position, such as at position 9.

Fatty acid: the term “fatty acid” refers herein to a carboxylic acid having a long aliphatic chain, i.e. an aliphatic chain between 13 and 28 carbon atoms, such as 13, 14, 15, 16, 17, 18, 19, 20, 21, 22, 23, 24, 25, 26, 27 or 28 carbon atoms. Most naturally occurring fatty acids are linear, i.e. unbranched. They can be saturated, or desaturated.

Fatty acyl-CoA: the term will herein be used interchangeably with “fatty acyl-CoA ester”, and refers to compounds of general formula R—CO-SCoA, where R is a fatty carbon chain having a carbon chain length of 13 to 28 carbon atoms, such as 13, 14, 15, 16, 17, 18, 19, 20, 21, 22, 23, 24, 25, 26, 27 or 28 carbon atoms. The fatty carbon chain is joined to the —SH group of CoA by a thioester bond. Fatty acyl-CoAs can be saturated or desaturated, depending on whether the fatty acid which it is derived from is saturated or desaturated.

Fatty alcohol: the term “fatty alcohol” refers herein to an alcohol derived from a fatty acyl-CoA, having a carbon chain length of 13 to 28 carbon atoms, such as 13, 14, 15, 16, 17, 18, 19, 20, 21, 22, 23, 24, 25, 26, 27 or 28 carbon atoms. Fatty alcohols can be saturated or desaturated.

Fatty alcohol acetate: the term refers to an acetate having a fatty carbon chain, i.e. an aliphatic chain between 13 and 28 carbon atoms, such as 13, 14, 15, 16, 17, 18, 19, 20, 21, 22, 23, 24, 25, 26, 27 or 28 carbon atoms. Fatty acyl acetates can be saturated or desaturated.

Fatty aldehyde: the term refers herein to an aldehyde derived from a fatty acyl-CoA, having a carbon chain length of 13 to 28 carbon atoms, such as 13, 14, 15, 16, 17, 18, 19, 20, 21, 22, 23, 24, 25, 26, 27 or 28 carbon atoms. Fatty aldehydes can be saturated or desaturated.

Functional variant: the term refers herein to functional variants of an enzyme, which retain at least some of the activity of the parent enzyme. Thus, a functional variant of an acyl-CoA oxidase, a desaturase, an alcohol-forming fatty acyl-CoA reductase, an alcohol dehydrogenase, an aldehyde-forming fatty acyl-CoA reductase, an acetyltransferase, or an NAD(P)H cytochrome b5 oxidoreductase (Ncb5or) can catalyse the same conversion as the acyl-CoA oxidase, the desaturase, the alcohol-forming fatty acyl-CoA reductase, the alcohol dehydrogenase, the aldehyde-forming fatty acyl-CoA reductase, or the acetyltransferase, respectively, from which they are derived, although the efficiency of reaction may be different, e.g. the efficiency is decreased or increased compared to the parent enzyme or the substrate specificity is modified.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous Δ9 desaturase” when applied to Saccharomyces cerevisiae refers to a Δ9 desaturase which is not naturally present in a wild type S. cerevisiae cell, e.g. a Δ9 desaturase derived from Drosophila melanogaster.

Identity/similarity: the terms identity and similarity, with respect to a polynucleotide (or polypeptide), are defined herein as the percentage of nucleic acids (or amino acids) in the candidate sequence that are identical or similar, respectively, to the residues of a corresponding native nucleic acids (or amino acids), after aligning the sequences and introducing gaps, if necessary, to achieve the maximum percent identity/similarity, and considering any conservative substitutions according to the NCIUB rules (https://iubmb.qmul.ac.uk/misc/naseq.html; NC-IUB, Eur J Biochem (1985)) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity or similarity. Methods and computer programs for the alignments are well known in the art. Generally, a given similarity between two sequences implies that the identity between these sequences is at least equal to the similarity; for example, if two sequences are 70% similar to one another, they cannot be less than 70% identical to one another—but could be sharing 80% identity.

Increased activity: the term “increased activity” herein refers to an increase in activity of a given peptide, such as a protein or an enzyme. The increase in activity can be measured using methods known in the art, such as for example using enzyme assays to measure the increase in activity of an enzyme. In some cases, the increase in activity results in higher production of the compound or compounds which the enzyme is generating, i.e. the product. Thus, increased activity of an enzyme may be measured by measuring the amount, such as the concentration, of said product. If an enzyme has increased activity, the concentration of product will be higher compared the concentration of product generated in similar or identical conditions by the same enzyme which does not have increased activity, e.g. the parent enzyme or unmodified enzyme. If the enzyme with increased activity is expressed inside a cell, the product can be measured as the product titer, i.e. the amount of product said cell has produced, and can be compared to the titer or amount of the same product obtained in similar or identical conditions from a cell expressing the parent or unmodified enzyme but otherwise having an identical or similar genotype as the cell expressing the enzyme with increased activity.

n double bonds: the term “n double bonds” used herein refer to the number of double bonds present in a fatty acyl-CoA. A fatty acyl-CoA having n double bonds has 0-3 double bond(s). Thus, a fatty acyl-CoA having n double bonds can be a saturated fatty acyl-CoA having 0 double bonds or a desaturated fatty acyl-CoA having one or more double bonds, such as 1, 2 or 3 double bonds. The n double bonds may be at any position other than position 12.

n′ double bonds: the term “n′ double bonds” as used herein refers to the number of double bonds present in a fatty acyl-CoA. A fatty acyl-CoA having n′ double bonds has 1-4 double bond(s). Thus a fatty acyl-CoA having n′ double bonds can be a desaturated fatty acyl-CoA having one or more double bonds, such as 1, 2, 3 or 4 double bonds, whereof one of the double bonds is at position 12.

Native: the term “native” when referring to a polypeptide, such as a protein or an enzyme, or to a polynucleotide, shall herein be construed to refer to a polypeptide or a polynucleotide which is naturally present in a wild type cell.

Pest: as used herein, the term ‘pest’ shall refer to an organism, in particular an animal such as an insect, detrimental to humans or human concerns, in particular in the context of agriculture or livestock production. A pest is any living organism which is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

Pheromone: pheromones are naturally occurring compounds. Lepidopteran pheromones are designated by an unbranched aliphatic chain (between 9 and 18 carbons, such as 9, 10, 11, 12, 13, 14, 15, 16, 17 or 18 carbon atoms) ending in an alcohol, aldehyde or acetate functional group and containing up to 3 double bonds in the aliphatic backbone, such as 1, 2 or 3 double bonds. Thus, desaturated fatty alcohols, desaturated fatty aldehydes and desaturated fatty alcohol acetates are typically comprised in pheromones. Pheromone compositions may be produced chemically or biochemically, for example as described herein. Pheromones thus comprise desaturated fatty alcohols, desaturated fatty aldehydes and/or desaturated fatty alcohol acetates, such as can be obtained by the methods and cells described herein.

“PidZ11” and “Pid12”: The 012 desaturase as set forth in SEQ ID NO: 2 was initially given the internal name “PidZ11” before it was discovered that the enzyme has Δ12 activity. Hence, a more appropriate name might be “Pid12”. The terms PidZ11” and “Pid12” are used interchangeable herein.

Reduced activity: the term “reduced activity” may herein refer to a total or a partial loss of activity of a given peptide, such as a protein or an enzyme. In some cases, peptides are encoded by essential genes, which cannot be deleted. In these cases, activity of the peptide can be reduced by methods known in the art, such as down-regulation of transcription or translation, inhibition of the peptide. In other cases, the peptide is encoded by a non-essential gene, and the activity may be reduced or it may be completely lost, e.g. as a consequence of a deletion of the gene encoding the peptide. In order to reduce, whether partially or totally, the activity of a given peptide, methods known in the art include not only mutations in the genes encoding said peptide, but also mutation of genes encoding regulatory factors involved in transcription or translation of the gene encoding said peptide, e.g. mutation of transcription factor genes or of transcription repressor genes resulting in increased or decreased expression of said transcription factors or repressors, which in turn reduce transcription levels from the gene encoding the peptide; truncation or mutation of the native promoter of the gene, for example to remove transcription factor binding sites or to render them inaccessible to said transcription factors; replacement of the native promoter with a weaker promoter, leading to reduced transcription of the coding sequence encoding the peptide; truncation or mutation of the native terminator of the gene, or replacement of the native terminator of the gene with another terminator sequence; mutation of the Kozak sequence. Other methods involve regulation at the RNA level, and include RNA interference systems such as Dicer or Argonaute, RNA silencing methods, introduction of CRISPR/Cas systems resulting in targeted RNA degradation. Regulation at the protein level is also envisaged, e.g. by using inhibitors or protein degradation sequences. The listed methods may be inducible, i.e. they may be activated in a transient manner as known in the art.

Saturated: the term “saturated” refers to a compound which is devoid of double or triple carbon-carbon bonds.

Specificity: the specificity of an enzyme towards a given substrate is the preference exhibited by this enzyme to catalyse a reaction starting from said substrate. In the present disclosure, a desaturase and/or a fatty acyl-CoA reductase having a higher specificity towards tetradecanoyl-CoA (myristoyl-CoA) than towards hexadecanoyl-CoA (palmitoyl-CoA) preferably catalyse a reaction with tetradecanoyl-CoA than with hexadecanoyl-CoA as a substrate. Methods to determine the specificity of a desaturase or a fatty acyl-CoA reductase are known in the art. For example, specificity of a given desaturase in a given cell expressing it can be determined by incubating said cell in a solution comprising methyl myristate for up to 48 hours, followed by extraction and esterification of the products with methanol. The profiles of the resulting fatty acid methyl esters can then be determined by GC-MS. Desaturases with higher specificity towards myristoyl-CoA and low specificity towards palmitoyl-CoA, for example, will result in higher concentration of (Z)9-C14:Me than (Z)9-C16:Me. For example, specificity of a given reductase in a given cell can be determined by incubating cells that express said reductase in a solution comprising methyl ester of (Z)9-myristate for up to 48 hours, followed by extraction and analysis of the resulting fatty alcohols by GC-MS. Reductases with higher specificity towards (Z)9-C14:CoA and low specificity towards (Z)9-C16:CoA will result in higher concentration of (Z)9-C14:OH than (Z)9-C16:OH.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

### Production of E12 Desaturated Compounds

The present inventors have discovered that expression of heterologous Δ12 desaturases in yeast cells results in the production of fatty acyl-CoA having a double bond at position 12. The heterologous Δ12 desaturases disclosed herein are capable of introducing a double bond at position 12, and optionally a double bond at a position other than position 12, in desaturated or saturated fatty acyl-CoAs having a carbon chain length of at least 13. The starting compound in which the desaturase may introduce the double bond at position 12 may be a saturated compound, or a desaturated compound already comprising a double bond at a position which is not position 12. Preferably, the starting compound is a desaturated fatty acyl-CoA comprising a double bond at position 9. The Δ12 desaturase may be able to introduce additional double bonds besides the double bond at position 12.

Thus, provided herein is a yeast cell capable of producing a desaturated fatty acyl-CoA having n′ double bond(s), wherein at least one of said double bond(s) is at position 12, wherein said yeast cell expresses a Δ12 heterologous fatty acyl-CoA desaturase, said desaturase being capable of introducing a double bond at position 12 in a saturated or desaturated fatty acyl-CoA having a carbon chain length of at least 13 and having n double bond(s),
wherein n and n′ are integers,
wherein 0≤n≤3 and wherein 1≤n′≤4.

Also provided herein is a yeast cell expressing a heterologous Δ12 desaturase, said desaturase being capable of introducing a double bond at position 12 in a saturated or desaturated fatty acyl-CoA having a carbon chain length of at least 13 and having n double bond(s) thereby producing a desaturated fatty acyl-CoA having n′ double bond(s), wherein at least one of said double bond(s) is at position 12,
wherein n and n′ are integers,
wherein 0≤n≤3 and wherein 1≤n′≤4.

In preferred embodiments, the saturated or desaturated fatty acyl-CoA having a carbon chain length of at least 13 and having n double bond(s) is a desaturated fatty acyl-CoA having a carbon chain length of 14, such as (Z9)-tetradecenoyl-CoA.

Further provided herein is a yeast cell capable of producing (Z9, E12)-tetradecadienoyl-CoA, said yeast cell expressing
i) at least one further heterologous desaturase capable of introducing a first double bond at position 9 in tetradecanoyl-CoA, thereby converting said tetradecanoyl-CoA to (Z9)-tetradecenoyl-CoA; and
ii) at least one heterologous Δ12 desaturase capable of introducing a second double bond at position 12 in (Z9)-tetradecenoyl-CoA, thereby converting (Z9)-tetradecenoyl-CoA into (Z9, E12)-tetradecadienoyl-CoA.

Also provided herein is a yeast cell capable of producing a desaturated fatty acyl-CoA, said yeast cell expressing a Pid12 (SEQ ID NO: 2) desaturase, an EcauDes12 (SEQ ID NO: 55) desaturase, an Ee_d12 (SEQ ID NO: 87) desaturase or an Eku_d12 (SEQ ID NO: 85) desaturase capable of introducing a trans-double bond at position 12 in a fatty acyl-CoA, thereby converting the fatty acyl-CoA into a desaturated E12-fatty acyl-CoA. The EcauDes12 desaturase is also known as Ecau_D4 (Ding et al., 2021).

### Desaturase

In the present invention, the terms ‘fatty acyl-CoA desaturase’, ‘desaturase’, ‘fatty acyl desaturase’ and ‘FAD’ will be used interchangeably. The term generally refers to an enzyme capable of introducing at least one double bond in E/Z confirmations in an acyl-CoA having a chain length of 8, 9, 10, 11, 12, 13, 14, 15, 16, 17, 18, 19, 20, 21 or 22 carbon atoms. The double bond may be introduced in any position. For example, a desaturase introducing a double bond in position 12 is termed Δ12 desaturase. In some embodiments, the desaturases disclosed herein are capable of introducing double bonds at several positions of a fatty acyl-CoA, such as for example at position 12 and at position 9.

Desaturases catalyse the reaction:

$$Fatty acyl - CoA + 2 ferrocytochrome b 5 + O ( 2 ) + 2 H ( + ) <= > \text{} desaturated fatty acyl - CoA + 2 ferricytochrome b 5 + 2 H ( 2 ) O$$

Provided herein are yeast cells expressing heterologous Δ12 desaturases and optionally other heterologous desaturases.

### Δ12 Desaturase

Disclosed herein is a Δ12 desaturase capable of introducing a double bond at position 12 of a fatty acyl-CoA having a carbon chain length of 13 or more, which when expressed in a yeast cell allows said yeast cell to produce a desaturated fatty acyl-CoA having a double bond in position 12. The heterologous Δ12 desaturase may be capable of introducing a double bond at position 12 in a desaturated or saturated fatty acyl-CoA. In other words, the Δ12 desaturase may be capable of introducing a double bond at position 12 of a fatty acyl-CoA having no double bond, or one or more double bonds at one or more positions other than position 12, for example at one or more positions selected from the group consisting of position 8, 9, 10, 11, 13, 14, 15, 16, 17, 18, 19, 20 and 21. In one embodiment, the Δ12 desaturase is capable of introducing a double bond at position 12 of a fatty acyl-CoA having no double bond or having at least one double bond at position 9. In preferred embodiments, the heterologous Δ12 desaturase is capable of introducing a double bond at position 12 of a desaturated fatty acyl-CoA, such as a fatty acyl-CoA having a double bond at position 9.

In some embodiments, the Δ12 desaturase is capable of introducing at least one double bond at position 12 and is also capable of introducing at least one additional double bond at an additional position, such as at any other position than position 12, of a fatty acyl-CoA. In other words, the Δ12 desaturase may be capable of introducing a double bond at position 12 and at least one more position selected from the group consisting of position 8, 9, 10, 11, 13, 14, 15, 16, 17, 18, 19, 20 and 21. In one embodiment, the Δ12 desaturase is capable of introducing a double bond at position 12 and at position 9 of a fatty acyl-CoA.

In one embodiment, the heterologous Δ12 desaturase is native to an organism of a genus selected from the group consisting of Cadra, Plodia, Ephestia, Maliarpha, and Amorbia. In one embodiment, the heterologous Δ12 desaturase is native to an organism of a species selected from the group consisting of Cadra cautella, Ephestia elutella, Ephestia kuehniella, Plodia interpunctella, Maliarpha separatella, and Amorbia cuneana. In a preferred embodiment, the heterologous Δ12 desaturase is a Plodia desaturase, such as a Plodia interpunctella desaturase. Cadra cautella is also known and referred to as Ephestia cautella.

In one embodiment, the Δ12 desaturase is a Plodia Δ12 desaturase. In one embodiment, the desaturase is a Plodia interpunctella Δ12 desaturase, such as the Δ12 desaturase as set forth in SEQ ID NO: 2 (Pid12). In some embodiments, the Δ12 desaturase is a functional variant of a Plodia Δ12 desaturase, a functional variant of a Plodia interpunctella Δ12 desaturase, or a functional variant of the Δ12 desaturase as set forth in SEQ ID NO: 2 (Pid12), having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the Pid12 Δ12 desaturase as set forth in SEQ ID NO: 2. Without being bound by theory, the Pid12 Δ12 desaturase introduces a single double bond in a fatty acyl-CoA having a carbon chain length of at least 13, wherein said single double bond is at position 12.

The gene encoding the heterologous Δ12 desaturase may be codon-optimized for the yeast cell expressing the desaturase, as is known in the art. Methods to determine whether the desaturase is expressed in the cell are known to the person of skill in the art, and include for instance detection of a given product from a given substrate, as detailed herein above and as illustrated in the examples below.

Further provided herein is the use of a Plodia interpunctella desaturase in a method for introducing at least one double bond at position 12 in a saturated or desaturated fatty acyl-CoA. In some embodiments, the Plodia interpunctella desaturase introduces a single double bond in the fatty acyl-CoA, which is at position 12. In some embodiments, the Plodia interpunctella desaturase is Pid12 (SEQ ID NO: 2) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

In one embodiment, the method is performed in vitro, for example by providing a desaturase as described above, in particular a Pid12 desaturase as set forth in SEQ ID NO: 2, or a functional variant thereof having at least 70% similarity or identity thereto, and contacting said desaturase with a saturated or desaturated fatty acyl-CoA. The desaturase may be purified from a cell expressing said desaturase as is known in the art. In one embodiment, the method is performed in vivo, preferably in a yeast cell as defined herein in the section “Yeast cell”.

In one embodiment, the Δ12 desaturase is a Cadra Δ12 desaturase. In one embodiment, the desaturase is a Cadra cautella Δ12 desaturase, such as the Δ12 desaturase as set forth in SEQ ID NO: 55 (EcauDes12). In some embodiments, the Δ12 desaturase is a functional variant of a Cadra Δ12 desaturase, a functional variant of a Cadra cautella Δ12 desaturase, or a functional variant of the Δ12 desaturase as set forth in SEQ ID NO: 55 (EcauDes12), having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the EcauDes12 Δ12 desaturase as set forth in SEQ ID NO: 55. Without being bound by theory, the EcauDes12 Δ12 desaturase introduces a single double bond in a fatty acyl-CoA having a carbon chain length of at least 13, wherein said single double bond is at position 12.

Further provided herein is the use of a Cadra cautella desaturase in a method for introducing at least one double bond at position 12 in a saturated or desaturated fatty acyl-CoA. In some embodiments, the Cadra cautella desaturase introduces a single double bond in the fatty acyl-CoA, which is at position 12. In some embodiments, the Cadra cautella desaturase is SEQ ID NO: 55 (EcauDes12) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

In one embodiment, the method is performed in vitro, for example by providing a desaturase as described above, in particular an EcauDes12 desaturase as set forth in SEQ ID NO: 55, or a functional variant thereof having at least 70% similarity or identity thereto, and contacting said desaturase with a saturated or desaturated fatty acyl-CoA. The desaturase may be purified from a cell expressing said desaturase as is known in the art. In one embodiment, the method is performed in vivo, preferably in a yeast cell as defined herein in the section “Yeast cell”.

In one embodiment, the Δ12 desaturase is an Ephestia Δ12 desaturase. In one embodiment, the desaturase is an Ephestia kuehniella Δ12 desaturase, such as the Δ12 desaturase as set forth in SEQ ID NO: 85 (Eku_d12). In one embodiment, the desaturase is an Ephestia elutella Δ12 desaturase, such as the Δ12 desaturase as set forth in SEQ ID NO: 87 (Ee_d12). In some embodiments, the Δ12 desaturase is a functional variant of an Ephestia Δ12 desaturase, a functional variant of an Ephestia kuehniella Δ12 desaturase, a functional variant of an Ephestia elutella Δ12 desaturase, or a functional variant of the Δ12 desaturase as set forth in SEQ ID NO: 85 (Eku_d12) or SEQ ID NO: 87 (Ee_d12), having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the Eku_d12 Δ12 desaturase as set forth in SEQ ID NO: 85 or the Ee_d12 Δ12 desaturase as set forth in SEQ ID NO: 87. Without being bound by theory, the Eku_d12 Δ12 desaturase and the Ee_d12 Δ12 desaturase introduces a single double bond in a fatty acyl-CoA having a carbon chain length of at least 13, wherein said single double bond is at position 12.

Further provided herein is the use of an Ephestia kuehniella desaturase in a method for introducing at least one double bond at position 12 in a saturated or desaturated fatty acyl-CoA. In some embodiments, the Ephestia kuehniella desaturase introduces a single double bond in the fatty acyl-CoA, which is at position 12. In some embodiments, the Ephestia kuehniella desaturase is Eku_d12 (SEQ ID NO: 85) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

Further provided herein is the use of an Ephestia elutella desaturase in a method for introducing at least one double bond at position 12 in a saturated or desaturated fatty acyl-CoA. In some embodiments, the Ephestia elutella desaturase introduces a single double bond in the fatty acyl-CoA, which is at position 12. In some embodiments, the Ephestia elutella desaturase is Eku_d12 (SEQ ID NO: 87) or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

In one embodiment, the method is performed in vitro, for example by providing a desaturase as described above, in particular an Eku_d12 desaturase as set forth in SEQ ID NO: 85 or an Ee_d12 desaturase as set forth in SEQ ID NO: 87, or a functional variant thereof having at least 70% similarity or identity thereto, and contacting said desaturase with a saturated or desaturated fatty acyl-CoA. The desaturase may be purified from a cell expressing said desaturase as is known in the art. In one embodiment, the method is performed in vivo, preferably in a yeast cell as defined herein in the section “Yeast cell”.

The present yeast cells may express at least one heterologous Δ12 desaturase. In some embodiments, the cell expresses one heterologous Δ12 desaturase. It may however be desirable to express several heterologous Δ12 desaturases, such as at least two heterologous Δ12 desaturases, which may be identical or different. Alternatively, it may be desirable to express several copies of the nucleic acid encoding the at least one heterologous Δ12 desaturase, such as at least two copies, such as at least three copies, or more. In other embodiments, the cell expresses at least two heterologous Δ12 desaturases, for example three heterologous Δ12 desaturases.

In some embodiments, the Δ12 desaturase is a functional variant of a Δ12 desaturase which comprises amino acid sequences from at least two different proteins, such as two different desaturases, such as three different desaturases. Such functional variant may for example be a fusion protein, such as a mosaic or chimeric protein, of a Δ12 desaturase. Preferably, at least one of the amino acid sequences comprised in such functional variant of a Δ12 desaturase is derived from a Δ12 desaturase. Thus, in some embodiments, the Δ12 desaturase disclosed herein is a functional variant of a Δ12 desaturase comprising or consisting of an amino acid sequence from a Δ12 desaturase and at least one amino acid sequence, such as two amino acid sequences, or more, from at least one other desaturase, such as a Δ9 desaturase. In other embodiments, the functional variant of the Δ12 desaturase comprises or consists of an amino acid sequence from a Δ12 desaturase and at least two amino acid sequences from at least one other desaturase, such as a Δ9 desaturase, a Δ11 desaturase, two Δ9 desaturases, two Δ11 desaturases, or more. In further other embodiments, the Δ12 desaturase disclosed herein is a functional variant of a Δ12 desaturase comprising an amino acid sequence derived from a Δ12 desaturase and an amino acid sequence derived from another Δ12 desaturase, such that the functional variant of the Δ12 desaturase comprises or consists of amino acid sequences from two different Δ12 desaturases.

In some embodiments, the Δ12 desaturase and the at least one other desaturase, such as the Δ9, Δ11 and/or Δ12 desaturase, are derived from the same species. In other words, the functional variant of the Δ12 desaturase comprises or consists of amino acid sequences from a Δ12 desaturase and at least one other desaturase which are native to the same species, such as an amino acid sequence from a Δ12 desaturase and an amino acid sequence from a Δ11 desaturase, wherein said Δ12 desaturase and Δ11 desaturase are both derived from Plodia interpunctella. In other embodiments, the Δ12 desaturase and the at least one other desaturase are derived from two different species, three different species, or more different species. In other words, the functional variant of the Δ12 desaturase comprises or consists of amino acid sequences from a Δ12 desaturase and at least one another desaturase which are native to at least two different species, such as an amino acid sequence from a Plodia interpunctella Δ12 desaturase and an amino acid sequence from a Cadra cautella Δ12 desaturase.

In some embodiments, the functional variant of the Δ12 desaturase comprises or consists of an amino acid sequence from one desaturase, wherein at least one of the termini of said one desaturase has been replaced, such as exchanged with a termini of another desaturase, such as the corresponding termini of another desaturase. The corresponding termini of the N-terminus of said one desaturase herein refers to the N-terminus of said another desaturase, and the same terminology applies to the C-terminus. The skilled person will understand that the termini of many enzymes, such as that of desaturases, may be changed with respect to their native sequences, while the function of said enzymes are preserved, i.e. without the enzymes loose its activity. In contrast, changing the residues comprising the catalytic site and/or residues of importance to the catalytic activity of an enzyme may not be possible without disturbing or changing the function and/or catalytic activity of said enzyme. With regards to the termini, the skilled person will understand that it may be important to maintain the flexibility, hydrophobicity and/or hydrophilicity of the termini in order to preserve the features and/or function of the enzyme. For example, if the enzyme is anchored, also known as embedded and/or inserted, such as to a membrane, via at least one of its termini, if may be important to maintain the same or a similar hydrophobicity of its terminus.

Thus, in some embodiments, the functional variant of the Δ12 desaturase comprises or consists of an amino acid sequence from a Δ12 desaturase that is stripped from its native N- and/or C-terminus, and fused to the N- and/or C-terminus of another desaturase, such as another Δ12 desaturase, Δ9 desaturase or Δ11 desaturase. In some embodiments, the functional variant of the Δ12 desaturase comprises or consists of a part of the amino acid sequence of a Δ12 desaturase, wherein 60 N-terminus amino acid residues of said Δ12 desaturase have been deleted or substituted by an amino acid sequence from at least one other desaturase, such as wherein 50 N-terminus amino acid residues of said Δ12 desaturase have been deleted or substituted by an amino acid sequence from at least one other desaturase, such as wherein 40 N-terminus amino acid residues of said Δ12 desaturase have been deleted or substituted by an amino acid sequence from at least one other desaturase, such as wherein 35 N-terminus amino acid residues of said Δ12 desaturase have been deleted or substituted by an amino acid sequence from at least one other desaturase, such as wherein 30 N-terminus amino acid residues of said Δ12 desaturase have been deleted or substituted by an amino acid sequence from at least one other desaturase, such as wherein 25 N-terminus amino acid residues of said Δ12 desaturase have been deleted or substituted by an amino acid sequence from at least one other desaturase, such as wherein 20 N-terminus amino acid residues of said Δ12 desaturase have been deleted or substituted by an amino acid sequence from at least one other desaturase, such as wherein 15 N-terminus amino acid residues of said Δ12 desaturase have been deleted or substituted by an amino acid sequence from at least one other desaturase, such as wherein 10 N-terminus amino acid residues of said Δ12 desaturase have been deleted or substituted by an amino acid sequence from at least one other desaturase, such as wherein 5 N-terminus amino acid residues of said Δ12 desaturase have been deleted or substituted by an amino acid sequence from at least one other desaturase, such as wherein between 1 and 55 N-terminus amino acid residues of said Δ12 desaturase have been deleted or substituted by an amino acid sequence from at least one other desaturase, such as wherein between 5 and 45 N-terminus amino acid residues of said Δ12 desaturase have been deleted or substituted by an amino acid sequence from at least one other desaturase, such as wherein between 10 and 40 N-terminus amino acid residues of said Δ12 desaturase have been deleted or substituted by an amino acid sequence from at least one other desaturase, such as wherein between 15 and 35 N-terminus amino acid residues of said Δ12 desaturase have been deleted or substituted by an amino acid sequence from at least one other desaturase, such as wherein between 20 and 35 N-terminus amino acid residues of said Δ12 desaturase have been deleted or substituted by an amino acid sequence from at least one other desaturase, such as wherein between 25 and 35 N-terminus amino acid residues of said Δ12 desaturase have been deleted or substituted by an amino acid sequence from at least one other desaturase,

and/or
wherein 50 C-terminus amino acid residues of said Δ12 desaturase have been deleted or substituted by an amino acid sequence from at least one other desaturase, such as wherein 40 C-terminus amino acid residues of said Δ12 desaturase have been deleted or substituted by an amino acid sequence from at least one other desaturase, such as wherein 35 C-terminus amino acid residues of said Δ12 desaturase have been deleted or substituted by an amino acid sequence from at least one other desaturase, such as wherein 30 C-terminus amino acid residues of said Δ12 desaturase have been deleted or substituted by an amino acid sequence from at least one other desaturase, such as wherein 25 C-terminus amino acid residues of said Δ12 desaturase have been deleted or substituted by an amino acid sequence from at least one other desaturase, such as wherein 20 C-terminus amino acid residues of said Δ12 desaturase have been deleted or substituted by an amino acid sequence from at least one other desaturase, such as wherein 15 C-terminus amino acid residues of said Δ12 desaturase have been deleted or substituted by an amino acid sequence from at least one other desaturase, such as wherein 10 C-terminus amino acid residues of said Δ12 desaturase have been deleted or substituted by an amino acid sequence from at least one other desaturase, such as wherein between 1 and 55 C-terminus amino acid residues of said Δ12 desaturase have been deleted or substituted by an amino acid sequence from at least one other desaturase, such as wherein between 5 and 45 C-terminus amino acid residues of said Δ12 desaturase have been deleted or substituted by an amino acid sequence from at least one other desaturase, such as wherein between 10 and 40 C-terminus amino acid residues of said Δ12 desaturase have been deleted or substituted by an amino acid sequence from at least one other desaturase, such as wherein between 15 and 35 C-terminus amino acid residues of said Δ12 desaturase have been deleted or substituted by an amino acid sequence from at least one other desaturase, such as wherein between 20 and 35 C-terminus amino acid residues of said Δ12 desaturase have been deleted or substituted by an amino acid sequence from at least one other desaturase, such as wherein between 25 and 35 C-terminus amino acid residues of said Δ12 desaturase have been deleted or substituted by an amino acid sequence from at least one other desaturase. In preferred embodiments, said Δ12 desaturase is selected from the Δ12 desaturases set forth in SEQ ID NO: 2 (Pid12), SEQ ID NO: 55 (EcauDes12), SEQ ID NO: 85 (Eku_d12) or SEQ ID NO: 87 (Ee_d12).

Preferably, in the functional variant of the Δ12 desaturase, the C- or N-terminus residues which are deleted are substituted with an equal or near equal amount of amino acid residues from the other desaturase, such that for example in a functional variant of a Δ12 desaturase where 30 N-terminus amino acids have been deleted, said 30 amino acids have been substituted with in the range of 20 to 40 N-terminus amino acid residues from another desaturase, such as in the range of 25 to 35 N-terminus amino acid residues from another desaturase, such as in the range of 28 to 32 N-terminus amino acid residues from another desaturase, such as from another Δ11 desaturase. Thus, for example in a functional variant of a Δ12 desaturase where 30 C-terminus amino acids have been deleted, said 30 amino acids have been substituted with in the range of 20 to 40 C-terminus amino acid residues from another desaturase, such as in the range of 25 to 35 C-terminus amino acid residues from another desaturase, such as in the range of 28 to 32 C-terminus amino acid residues from another desaturase, such as from another Δ11 desaturase.

The N-terminus residues are herein defined as the first residues of the amino acid sequence of a protein, such that deleting or replacing X amino acid residues of the N-terminus of a protein refers to deleting or replacing the X first amino acids of said protein. In other words, deleting or replacing 30 amino acid residues of the N-terminus of a protein of 300 amino acids refers to deleting or replacing amino acid number 1 to 30 of said protein.

The C-terminus residues are herein defined as the last residues of the amino acid sequence of a protein, such that deleting or replacing X amino acid residues of the C-terminus of a protein refers to deleting or replacing the X last amino acids of said protein. In other words, deleting or replacing 30 amino acid residues of the C-terminus of a protein of 300 amino acids refers to deleting or replacing amino acid number 271 to 300 of said protein.

In some embodiments, the functional variant of the Δ12 desaturase comprises or consists of an amino acid sequence from a desaturase, such as SEQ ID NO: 2 (Pid12), SEQ ID NO: 55 (EcauDes12), SEQ ID NO: 85 (Eku_d12) or SEQ ID NO: 87 (Ee_d12), wherein at least one amino acid residue has been deleted, inserted or substituted with another amino acid residue. The deletion, insertion, or substitution may be found in the C-terminus or N-terminus part of the sequence.

In some embodiments, at least one of the N-terminus amino acid residues 1 to 50, such as 1 to 40, such as 1 to 30, such as 1 to 20, of the sequence, such as of a sequence selected from SEQ ID NO: 2 (Pid12), SEQ ID NO: 55 (EcauDes12), SEQ ID NO: 85 (Eku_d12) and SEQ ID NO: 87 (Ee_d12), have been deleted, inserted or substituted with another amino acid residue.

In some embodiments, at least one of the C-terminus amino acid residues 250 to 400, such as 260 to 390, such as 270 to 380, such as 280 to 370, such as 290 to 350, such as 300 to 350, of the sequence, such as of a sequence selected from SEQ ID NO: 2 (Pid12), SEQ ID NO: 55 (EcauDes12), SEQ ID NO: 85 (Eku_d12) and SEQ ID NO: 87 (Ee_d12), have been deleted, inserted or substituted with another amino acid residue. In some embodiments, at least one of amino acid residues 290 to 400, such as 290 to 390, such as 290 to 380, such as 290 to 370, such as 290 to 350, ofthe sequence, such as SEQ ID NO: 2 (Pid12), SEQ ID NO: 55 (EcauDes12), SEQ ID NO: 85 (Eku_d12) and SEQ ID NO: 87 (Ee_d12), have been deleted, inserted or substituted with another amino acid residue. In some embodiments, at least one of amino acid residues 302 to 348 of the sequence, such as SEQ ID NO: 2 (Pid12), SEQ ID NO: 55 (EcauDes12), SEQ ID NO: 85 (Eku_d12) and SEQ ID NO: 87 (Ee_d12), have been deleted, inserted or substituted with another amino acid residue.

In some embodiments, the functional variant of the Δ12 desaturase comprises or consists of an amino acid sequence from a Plodia interpunctella Δ12 desaturase, and an amino acid sequence from another Plodia interpunctella desaturase. In one embodiment, the functional variant of the Δ12 desaturase comprises or consists of a part of the amino acid sequence of the Plodia interpunctella desaturase Pid12 set forth in SEQ ID NO: 2, and part of the amino acid sequence of the Plodia interpunctella desaturase Desat65 set forth in SEQ ID NO: 47. In some embodiments, the functional variant of the Δ12 desaturase consists of a variant of Pid12 (SEQ ID NO: 2), wherein amino acid residues 1-30 of the N-terminus and/or amino acid residues 300-335 of the C-terminus have been substituted with amino acid residues 1-29 of the C-terminus and amino acid residues 299-344 of the N-terminus of Desat65 (SEQ ID NO: 47), respectively. Thus, in some embodiments, the functional variant of the Δ12 desaturase consists of the sequence set forth in SEQ ID NO: 80.

In some embodiments, the functional variant of the Δ12 desaturase comprises or consists of an amino acid sequence from a Cadra cautella Δ12 desaturase, and an amino acid from a Plodia interpunctella desaturase. In one embodiment, the functional variant of the Δ12 desaturase comprises or consists of a part of the amino acid sequence of the Cadra cautella desaturase EcauDes12 as set forth in SEQ ID NO: 55, and part of the amino acid sequence of the Plodia interpunctella desaturase Desat65 as set forth in SEQ ID NO: 47. In some embodiments, the functional variant of the Δ12 desaturase consists of a variant of EcauDes12 (SEQ ID NO: 55), wherein amino acid residues 1-30 of the N-terminus and amino acid residues 300-338 of the C-terminus have been substituted with amino acid residues 1-29 of the N-terminus and amino acid residues 299-344 of the C-terminus of Desat65 (SEQ ID NO: 47), respectively. Thus, in some embodiments, the functional variant of the Δ12 desaturase consists of the sequence set forth in SEQ ID NO: 81.

In some embodiments, the functional variant of the Δ12 desaturase comprises or consists of an amino acid sequence from a Cadra cautella Δ12 desaturase, and an amino acid from an Amyelois transitella Δ11 desaturase. In one embodiment, the functional variant of the Δ12 desaturase comprises or consists of part of the amino acid sequence of the Cadra cautella desaturase EcauDes12 as set forth in SEQ ID NO: 55, and part of the amino acid sequence of the Amyelois transitella Δ11 desaturase Desat16 as set forth in SEQ ID NO: 83. In some embodiments, the functional variant of the Δ12 desaturase consists of a variant of EcauDes12 (SEQ ID NO: 55), wherein amino acid residues 1-30 of the N-terminus and amino acid residues 300-338 of the C-terminus have been substituted with amino acid residues 1-20 of the N-terminus and amino acid residues 302-326 of the C-terminus of Desat16 (SEQ ID NO: 83), respectively. Thus, in some embodiments, the functional variant of the Δ12 desaturase consists of the sequence set forth in SEQ ID NO: 82.

### Further Desaturase

The yeast cell provided herein may, in addition to the heterologous Δ12 desaturase, express at least one further desaturase which is not a Δ12 desaturase. In some embodiments, the further desaturase is a homologous desaturase, such as a homologous Δ9 desaturase or a homologous Δ11 desaturase. In other words, the yeast cell expresses at least one native desaturase which is not a Δ12 desaturase. For example, the yeast cell may be a Yarrowia lipolytica yeast cell expressing the homologous Δ9 desaturase Ole1. In preferred embodiments, however, the further desaturase is a heterologous desaturase. In other words, the yeast cell expresses at least one further heterologous desaturase which is not a Δ12 desaturase.

In one embodiment, the further desaturase is a heterologous desaturase which is capable of introducing at least one double bond at any position which is not position 12 in a saturated or desaturated fatty acyl-CoA having a carbon chain length of at least 13. In other words, the further desaturase may be capable of introducing at least one double bond at a position selected from the group consisting of position 8, 9, 10, 11, 13, 14, 15, 16, 17, 18, 19, 20 and 21 in a fatty acyl-CoA. In a preferred embodiment, said fatty acyl-CoA is a saturated fatty acyl-CoA, for example a tetradecanoyl-CoA. In another embodiment, said fatty acyl-CoA is a desaturated fatty acyl-CoA having a double bond at position 12. In yet another embodiment, said fatty acyl-CoA is a desaturated fatty acyl-CoA having a double bond at position 12 and at a position other than position 12, such as for example at position 12 and at position 9.

Thus, in one embodiment, the yeast cell is capable of expressing at least one further heterologous desaturase which is a heterologous Δ5 desaturase. In another embodiment, the yeast cell is capable of expressing at least one further heterologous desaturase which is a heterologous Δ6 desaturase. In another embodiment, the yeast cell is capable of expressing at least one further heterologous desaturase which is a heterologous Δ7 desaturase. In another embodiment, the yeast cell is capable of expressing at least one further heterologous desaturase which is a heterologous M8 desaturase. In another embodiment, the yeast cell is capable of expressing at least one further heterologous desaturase which is a heterologous Δ9 desaturase. In another embodiment, the yeast cell is capable of expressing at least one further heterologous desaturase which is a heterologous Δ10 desaturase. In another embodiment, the yeast cell is capable of expressing at least one further heterologous desaturase which is a heterologous Δ11 desaturase. In another embodiment, the yeast cell is capable of expressing at least one further heterologous desaturase which is a heterologous Δ13 desaturase. In another embodiment, the yeast cell is capable of expressing at least one further heterologous desaturase which is a heterologous Δ14 desaturase. In another embodiment, the yeast cell is capable of expressing at least one further heterologous desaturase which is a heterologous Δ15 desaturase. In another embodiment, the yeast cell is capable of expressing at least one further heterologous desaturase which is a heterologous Δ16 desaturase. In another embodiment, the yeast cell is capable of expressing at least one further heterologous desaturase which is a heterologous Δ17 desaturase. In another embodiment, the yeast cell is capable of expressing at least one further heterologous desaturase which is a heterologous Δ18 desaturase. In another embodiment, the yeast cell is capable of expressing at least one further heterologous desaturase which is a heterologous Δ19 desaturase. In another embodiment, the yeast cell is capable of expressing at least one further heterologous desaturase which is a heterologous Δ20 desaturase. In another embodiment, the yeast cell is capable of expressing at least one further heterologous desaturase which is a heterologous Δ21 desaturase. In preferred embodiments, the further desaturase is a Δ9 desaturase or a Δ11 desaturase. Thus, the further desaturase may be capable of introducing a double bond in position 9 or position 11, respectively.

The further heterologous desaturase may be native to any type of organism which is different from the yeast cell in which it is expressed according to the present methods. In some embodiments, the heterologous desaturase is native to a plant, such as Ricinus communis or Pelargonium hortorum. In another embodiment, the heterologous desaturase is native to an insect, such as of the Diptera, the Coleoptera, or the Lepidoptera order, such as of the genus Agrotis, Argyrotaenia, Amyelois, Chauliognathus, Choristoneura, Cydia, Dendrophilus, Drosophila, Epiphyas, Grapholita, Helicoverpa, Lampronia, Lobesia, Manducta, Ostrinia, Pectinophora, Plutella, Thalassiosira, Thaumetopoea, Tribolium, Trichoplusia or Spodoptera, such as Agrotis segetum, Argyrotaenia velutiana, Amyelois transitella, Chauliognathus lugubris, Choristoneura parallela, Choristoneura rosaceana, Cydia pomonella, Dendrophilus punctatus, Drosophila ananassae, Drosophila grimshawi, Drosophila melanogaster, Drosophila mojavensis, Drosophila pseudoobscura, Drosophila virilis, Drosophila yakuba, Epiphyas postvittana, Grapholita molesta, Helicoverpa assulta, Helicoverpa zea, Lampronia capitella, Lobesia botrana, Manducta sexta, Ostrinia furnacalis, Ostrinia nubilalis, Pectinophora gossypiella, Plutella xylostella, Spodoptera exigua, Spodoptera littoralis, Spodoptera litura, Thalassiosira pseudonana, Thaumetopoea pityocampa, Tribolium castaneum or Trichoplusia ni.

In a preferred embodiment, the further heterologous desaturase is a Drosophila desaturase, such as a Drosophila melanogaster desaturase, a Drosophila virilis desaturase, a Drosophila grimshawi desaturase, a Drosophila yakuba desaturase, a Drosophila mojavensis desaturase, a Drosophila pseudoobscura desaturase, or a Drosophila ananassae desaturase.

In one embodiment, the further heterologous desaturase is a Drosophila desaturase. In one embodiment, the desaturase is a Drosophila virilis desaturase, such as the desaturase as set forth in SEQ ID NO: 4 (Desat61). In one embodiment, the desaturase is a Drosophila melanogaster desaturase, such as the desaturase as set forth in SEQ ID NO: 6 (Desat24). In one embodiment, the desaturase is a Drosophila grimshawi desaturase, such as the desaturase set forth in SEQ ID NO: 8 (Desat59). In one embodiment, the desaturase is a Drosophila yakuba desaturase, such as the desaturase as set forth in SEQ ID NO: 57 (Desat56). In one embodiment, the desaturase is a Drosophila ananassae desaturase, such as the desaturase as set forth in SEQ ID NO: 59 (Desat60). In some embodiments, the desaturase is a functional variant of a Drosophila desaturase, a functional variant of a Drosophila virilis desaturase, a functional variant of a Drosophila melanogaster desaturase, a functional variant of a Drosophila grimshawi desaturase, a functional variant of the desaturase as set forth in SEQ ID NO: 6 (Desat24), a functional variant of the desaturase as set forth in SEQ ID NO: 4 (Desat61), a functional variant of the desaturase as set forth in SEQ ID NO: 59 (Desat60), a functional variant of the desaturase as set forth in SEQ ID NO: 57 (Desat56), or a functional variant of the desaturase as set forth in SEQ ID NO: 8 (Desat59), having at least 60% identity or similarity thereto.

In one embodiment, the further heterologous desaturase is an Amyelois desaturase. In one embodiment, the desaturase is an Amyelois transitella desaturase, such as the desaturase as set forth in SEQ ID NO: 10 (Desat17) or SEQ ID NO: 12 (Desat18). In some embodiments, the desaturase is a functional variant of an Amyelois desaturase, a functional variant of an Amyelois transitella desaturase, a functional variant of the desaturase as set forth in SEQ ID NO: 10 (Desat17), or a functional variant of the desaturase as set forth in SEQ ID NO: 12 (Desat18), having at least 60% identity or similarity thereto.

In one embodiment, the further heterologous desaturase is a Choristoneura desaturase. In one embodiment, the desaturase is a Choristoneura parallela desaturase, such as the desaturase as set forth in SEQ ID NO: 61 (Desat74). In some embodiments, the desaturase is a functional variant of a Choristoneura desaturase, a functional variant of a Choristoneura parallela desaturase, or a functional variant of the desaturase as set forth in SEQ ID NO: 61 (Desat74), having at least 60% identity or similarity thereto.

In one embodiment, the further heterologous desaturase is a Spodoptera desaturase. In one embodiment, the desaturase is a Spodoptera litura desaturase, such as the desaturase as set forth in SEQ ID NO: 18 (Desat26). In some embodiments, the desaturase is a functional variant of a Spodoptera desaturase, a functional variant of a Spodoptera litura desaturase, or a functional variant of the desaturase as set forth in SEQ ID NO: 18 (Desat26), having at least 60% identity or similarity thereto.

In one embodiment, the further heterologous desaturase is a Saccharomyces desaturase. In one embodiment, the desaturase is a Saccharomyces cerevisiae desaturase, such as the desaturase as set forth in SEQ ID NO: 14 (Desat42). In some embodiments, the desaturase is a functional variant of a Saccharomyces desaturase, a functional variant of a Saccharomyces cerevisiae desaturase, or a functional variant of the desaturase as set forth in SEQ ID NO: 14 (Desat42), having at least 60% identity or similarity thereto.

In one embodiment, the further heterologous desaturase is a Yarrowia desaturase. In one embodiment, the desaturase is a Yarrowia lipolytica desaturase, such as the desaturase as set forth in SEQ ID NO: 16 (Desat69). In some embodiments, the desaturase is a functional variant of a Yarrowia desaturase, a functional variant of a Yarrowia lipolytica desaturase, or a functional variant of the desaturase as set forth in SEQ ID NO: 16 (Desat69), having at least 60% identity or similarity thereto.

The term “variant thereof having at least 60% identity or similarity” in relation to a given enzyme shall be understood to refer to variants having 60% identity or similarity or more to said enzyme, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity or similarity to the enzyme, or more.

The yeast cell to be modified may express a native desaturase, which may have a negative impact on the production of desaturated fatty acyl-CoA and/or desaturated fatty alcohol. Accordingly, if the cell to be modified expresses such a native desaturase, the organism may be modified so that activity of the native desaturase is reduced or absent.

To ensure lack or at least reduction of activity of a native desaturase, methods known in the art can be employed. The gene encoding the native desaturase may be deleted or partly deleted in order to ensure that the native desaturase is not expressed. Alternatively, the gene may be mutated so that the native desaturase is expressed but lacks activity, e.g. by mutation of the catalytic site of the enzyme. Alternatively, translation of mRNA to an active protein may be prevented by methods such as silencing RNA or siRNA. Alternatively, the cell may be incubated in a medium comprising an inhibitor which inhibits activity of the native desaturase. A compound inhibiting transcription of the gene encoding the native desaturase may also be provided so that transcription is inactivated when said compound is present. Other methods known in the art may be employed.

Inactivation of the native desaturase may thus be permanent or long-term, i.e. the modified cell exhibits reduced or no activity of the native desaturase in a stable manner, or it may be transient, i.e. the modified cell may exhibit activity of the native desaturase for periods of time, but this activity can be suppressed or reduced for other periods of time.

In some embodiments, the native yeast desaturase is replaced with a heterologous desaturase. The heterologous desaturase may for example be a desaturase which does not generate undesired by-products, or generates less undesired by-products compared to the native yeast desaturase when tested under the same conditions. Such undesired by-products may for example be (Z9)-hexadecenoyl-CoA (Z9-16:CoA) and/or (Z9)-hexadecenol (Z9-16:OH). I has been found that replacing the Yarrowia lipolytica OLE1 desaturase with a heterologous desaturase from Puccinia graminis or Arxula adeninivorans leads to reduced production of the by-product Z9-16:OH (Tsakraklides et al., 2018).

The heterologous desaturases replacing the native yeast desaturase may be specific to C18 compounds, such as octadecanoyl-CoA, and make octadecenoyl-CoA as the main product (Tsakraklides et al., 2018).

### Δ12 Desaturase and Further Desaturase

Any of the above Δ12 desaturases can be expressed together with any of the above further desaturases.

Thus, in some embodiments, the cell expresses:
a Plodia interpunctella Δ12 desaturase, for example Pid12 as set forth in SEQ ID NO: 2, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16;
   or functional variants thereof having at least 60% identity or similarity thereto.

In some embodiments, the cell expresses:
a Cadra cautella Δ12 desaturase, for example EcauDes12 as set forth in SEQ ID NO: 55, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16;
   or functional variants thereof having at least 60% identity or similarity thereto.

In some embodiments, the cell expresses:
an Ephestia kuehniella Δ12 desaturase, for example Eku_d12 as set forth in SEQ ID NO: 85, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16;
   or functional variants thereof having at least 60% identity or similarity thereto.

In some embodiments, the cell expresses:
an Ephestia elutella Δ12 desaturase, for example Ee_d12 as set forth in SEQ ID NO: 87, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16;
   or functional variants thereof having at least 60% identity or similarity thereto.

It will be understood that a functional variant of a desaturase having at least 60% identity or similarity to a given desaturase as above may have at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the desaturase.

### Increased C14 Specificity

Many desirable pheromone compounds have a carbon chain length of 14. It may thus be of interest to direct the reaction towards the production of C14 compounds. In some embodiments, the yeast cell disclosed herein expresses a desaturase having a higher specificity towards tetradecanoyl-CoA than towards hexadecanoyl-CoA and/or a fatty acyl-CoA reductase having a higher specificity towards desaturated tetradecanoyl-CoA (i.e. tetradecenoyl-CoA) than towards desaturated hexadecanoyl-CoA (i.e. hexadecanoyl-CoA). In other words, the desaturase is more specific for substrates having a carbon chain length of 14 than for substrates having a chain length of 16. Examples of yeast cells expressing such desaturases are disclosed in WO 2018/109167, in particular in the section entitled “Desaturase”.

Expression of such desaturases (and of any of the reductases described herein) in the yeast cell increases the fraction of total desaturated fatty alcohols having a carbon chain length of 14, particularly compared to the fraction of total desaturated fatty alcohols having a carbon chain length of 16. Desaturases which have the required specificity are in particular desaturases native to Drosophila, Spodoptera, Choristneura species, such as desaturases native to Drosophila melanogaster, Drosophila grimshawi, Drosophila virilis, Spodoptera litura, Choristoneura parallela or Choristoneura rosaceana, or variants thereof having at least 60% identity thereto.

In some embodiments, the desaturase is selected from the group consisting of:
i) a Δ9 desaturase having at least 60% identity to the Δ9 desaturase from Drosophila melanogaster as set forth in SEQ ID NO: 6;
ii) a desaturase having at least 60% identity to the Δ9 desaturase from Drosophila grimshawi as set forth in SEQ ID NO: 8;
iii) a desaturase having at least 60% identity to the Δ9 desaturase from Drosophila virilis as set forth in SEQ ID NO: 4;
iv) a Δ9 desaturase having at least 60% identity to the Δ9 desaturase from Spodoptera litura as set forth in SEQ ID NO: 18;
   and functional variants thereof having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity thereto.

These desaturases have been found to preferentially catalyse desaturation of C14 substrates when expressed in a yeast cell.

In such cells, the ratio of desaturated tetradecanoyl-CoA to desaturated hexadecanoyl-CoA is of at least 0.1, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

When the heterologous desaturase is expressed with a fatty acyl-CoA reductase, as described in detail below, the yeast cell produces desaturated fatty alcohols.

In such embodiments, the titre of desaturated fatty alcohols derived from the desaturated tetradecanoyl-CoA is of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, the titre of desaturated fatty alcohols derived from desaturated tetradecanoyl-CoA having a chain length of 14 is at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, desaturated fatty alcohols are yielded comprising at least 1% of a desaturated fatty alcohol having a chain length of 14, such as at least 1.5%, such as at least 2%, such as at least 2.5%, such as at least 3%, such as at least 3.5%, such as at least 4%, such as at least 4.5%, such as at least 5%, such as at least 7.5%, such as at least 10%, or more. In some embodiments, the yeast cell produces desaturated fatty alcohols having a range of carbon chain lengths, including desaturated fatty alcohols having a carbon chain length of 14 and desaturated fatty alcohols having a carbon chain length of 16. In such embodiments, the proportion of desaturated fatty alcohols having a carbon chain length of 14 relative to the sum of desaturated fatty alcohols having a carbon chain length of 14 and desaturated fatty alcohols having a carbon chain length of 16 is at least 5%, such as at least 7.5%, such as at least 10%, such as at least 15%, such as at least 20%, or more.

How to test whether a given desaturase has the required specificity can be done as described herein.

### Fatty Acyl-CoA Reductase

Further provided herein is a yeast cell expressing a fatty acyl-CoA reductase (FAR), said yeast cell being capable of converting at least a part of the (E12)-fatty acyl-CoA or (Z9, E12)-fatty acyl-CoA into the corresponding fatty alcohol. For example, the yeast cell may be capable of converting at least part of (Z9, E12)-tetradecadienoyl-CoA into (Z9, E12)-tetradecadien-1-ol.

The terms ‘fatty acyl-CoA reductase’ and ‘FAR’ will be used herein interchangeably. The term ‘heterologous FAR’ refers to a FAR which is not naturally expressed by the organism, such as by the yeast cell.

FARs catalyze the two-step reaction:

$$Acyl - CoA + 2 NADPH <= > CoA + alcohol + 2 NADP ( + )$$

wherein in a first step, the fatty acyl-CoA is reduced to a fatty aldehyde, before the fatty aldehyde is further reduced into a fatty alcohol in a second step. The fatty acyl-CoA may be a desaturated or a saturated fatty acyl-CoA.

The FARs capable of catalyzing such reaction are alcohol-forming fatty acyl-CoA reductases with an EC number 1.2.1.84.

In one embodiment, the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, and Yponomeuta, such as Agrotis ipsilon, Agrotis segetum, Amyelois transitella, Bicyclus anynana, Bombus lapidarius, Chilo suppressalis, Cydia pomonella, Helicoverpa armigera, Helicoverpa assulta, Heliothis subflexa, Heliothis virescens, Lobesia botrana, Ostrinia furnacalis, Ostrinia nubilalis, Ostrinia zag, Ostrinia zea, Plodia interpunctella, Plutella xylostella, Spodoptera exigua, Spodoptera frugiperda, Spodoptera littoralis, Spodoptera litura, Trichoplusia ni, Tyta alba, and Yponomeuta rorellus, especially Spodoptera exigua, Helicoverpa armigera, Spodoptera litura and Plodia interpunctella.

In one embodiment, the FAR is selected from the group consisting of FAR1 (SEQ ID NO: 20), FAR16 (SEQ ID NO: 22), FAR17 (SEQ ID NO: 24), FAR19 (SEQ ID NO: 26), FAR28 (SEQ ID NO: 28), FAR32 (SEQ ID NO: 30), FAR38 (SEQ ID NO: 32), FAR44 (SEQ ID NO: 63), FAR48 (SEQ ID NO: 65), FAR49 (SEQ ID NO: 67); FAR38 (SEQ ID NO: 32), FAR4 (SEQ ID NO: 69), FAR6 (SEQ ID NO: 71), FAR8 (SEQ ID NO: 73), FAR12 (SEQ ID NO: 75), FAR11 (SEQ ID NO: 77) or FAR5 (SEQ ID NO: 79), or functional variants thereof having at least 60% identity or similarity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity thereto.

In one embodiment, the heterologous FAR is an Agrotis FAR. In one embodiment, the FAR is an Agrotis segetum FAR, such as the FAR as set forth in SEQ ID NO: 75 (FAR12). In some embodiments, the FAR is a functional variant of an Agrotis FAR, or a functional variant of an Agrotis segetum FAR, such as a functional variant of the FAR set forth in SEQ ID NO: 75 (FAR12), having at least 60% identity thereto.

In one embodiment, the heterologous FAR is a Bicyclus FAR. In one embodiment, the FAR is a Bicyclus anynana FAR, such as the FAR as set forth in SEQ ID NO: 77 (FAR11). In some embodiments, the FAR is a functional variant of a Bicyclus FAR, or a functional variant of a Bicyclus anynana FAR, such as a functional variant of the FAR set forth in SEQ ID NO: 77 (FAR11), having at least 60% identity thereto.

In one embodiment, the heterologous FAR is a Helicoverpa FAR. In one embodiment, the FAR is a Helicoverpa armigera FAR, such as the FAR as set forth in SEQ ID NO: 20 (FAR1). In some embodiments, the FAR is a functional variant of a Helicoverpa FAR, or a functional variant of a Helicoverpa armigera FAR, such as a functional variant of the FAR set forth in SEQ ID NO: 20 (FAR1), having at least 60% identity thereto.

In one embodiment, the heterologous FAR is a Helicoverpa FAR. In one embodiment, the FAR is a Helicoverpa assulta FAR, such as the FAR as set forth in SEQ ID NO: 71 (FAR6). In some embodiments, the FAR is a functional variant of a Helicoverpa FAR, or a functional variant of a Helicoverpa assulta FAR, such as a functional variant of the FAR set forth in SEQ ID NO: 71 (FAR6), having at least 60% identity thereto.

In one embodiment, the heterologous FAR is a Heliothis FAR. In one embodiment, the FAR is a Heliothis subflexa FAR, such as the FAR as set forth in SEQ ID NO: 69 (FAR4). In some embodiments, the FAR is a functional variant of a Heliothis FAR, or a functional variant of a Heliothis subflexa FAR, such as a functional variant of the FAR set forth in SEQ ID NO: 69 (FAR4), having at least 60% identity thereto.

In one embodiment, the heterologous FAR is a Heliothis FAR. In one embodiment, the FAR is a Heliothis virescens FAR, such as the FAR as set forth in SEQ ID NO: 79 (FAR5). In some embodiments, the FAR is a functional variant of a Heliothis FAR, or a functional variant of a Heliothis virescens FAR, such as a functional variant of the FAR set forth in SEQ ID NO: 79 (FAR5), having at least 60% identity thereto.

In one embodiment, the heterologous FAR is an Ostrinia FAR. In one embodiment, the FAR is an Ostrinia furnacalis FAR, such as the FAR as set forth in SEQ ID NO: 63 (FAR44). In some embodiments, the FAR is a functional variant of an Ostrinia FAR, or a functional variant of an Ostrinia furnacalis FAR, such as a functional variant of the FAR set forth in SEQ ID NO: 63 (FAR44), having at least 60% identity thereto.

In one embodiment, the heterologous FAR is an Ostrinia FAR. In one embodiment, the FAR is an Ostrinia zag FAR, such as the FAR as set forth in SEQ ID NO: 65 (FAR48). In some embodiments, the FAR is a functional variant of an Ostrinia FAR, or a functional variant of an Ostrinia zag FAR, such as a functional variant of the FAR set forth in SEQ ID NO: 65 (FAR48), having at least 60% identity thereto.

In one embodiment, the heterologous FAR is an Ostrinia FAR. In one embodiment, the FAR is an Ostrinia zea FAR, such as the FAR as set forth in SEQ ID NO: 67 (FAR49). In some embodiments, the FAR is a functional variant of an Ostrinia FAR, or a functional variant of an Ostrinia zea FAR, such as a functional variant of the FAR set forth in SEQ ID NO: 67 (FAR49), having at least 60% identity thereto.

In one embodiment, the heterologous FAR is a Plodia FAR. In one embodiment, the FAR is a Plodia interpunctella FAR, such as the FAR as set forth in SEQ ID NO: 28 (FAR28) or SEQ ID NO: 30 (FAR32). In some embodiments, the FAR is a functional variant of a Plodia FAR, or a functional variant of a Plodia interpunctella FAR, such as a functional variant of the FAR set forth in SEQ ID NO: 28 (FAR28) or SEQ ID NO: 30 (FAR32), having at least 60% identity thereto.

In one embodiment, the heterologous FAR is a Spodoptera FAR. In one embodiment, the FAR is a Spodoptera exigua FAR, such as the FAR as set forth in SEQ ID NO: 22 (FAR16) or SEQ ID NO: 24 (FAR17). In one embodiment, the FAR is a Spodoptera litura FAR, such as the FAR as set forth in SEQ ID NO: 26 (FAR19). In some embodiments, the FAR is a functional variant of a Spodoptera FAR, a functional variant of a Spodoptera exigua FAR, a functional variant of a Spodoptera litura FAR, a functional variant of the FAR set forth in SEQ ID NO: 22 (FAR16), SEQ ID NO: 24 (FAR17) or a functional variant of the FAR set forth in SEQ ID NO: 26 (FAR19), having at least 60% identity thereto.

In one embodiment, the heterologous FAR is a Trichoplusia FAR. In one embodiment, the FAR is a Trichoplusia ni FAR, such as the FAR as set forth in SEQ ID NO: 32 (FAR38). In some embodiments, the FAR is a functional variant of a Trichoplusia FAR, or a functional variant of a Trichoplusia ni FAR, such as a functional variant of the FAR set forth in SEQ ID NO: 32 (FAR38), having at least 60% identity thereto.

In one embodiment, the heterologous FAR is an Yponomeuta FAR. In one embodiment, the FAR is an Yponomeuta rorellus FAR, such as the FAR as set forth in SEQ ID NO: 73 (FAR8). In some embodiments, the FAR is a functional variant of an Yponomeuta FAR, or a functional variant of an Yponomeuta rorellus FAR, such as a functional variant of the FAR set forth in SEQ ID NO: 73 (FAR8), having at least 60% identity thereto.

The term “variant thereof having at least 60% identity or similarity” in relation to a given enzyme shall be understood to refer to variants having 60% identity or similarity or more to said enzyme, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity or similarity to the enzyme, or more.

The present cells may express at least one heterologous FAR. In some embodiments, the cell expresses one heterologous FAR. It may however be desirable to express several heterologous FARs, such as at least two heterologous FARs, which may be identical or different. Alternatively, it may be desirable to express several copies of the nucleic acid encoding the at least one heterologous FAR, such as at least two copies, at least three copies or more. In other embodiments, the cell expresses at least two heterologous FARs, for example three heterologous FARs.

For example, the cell may express two copies of FAR1 or a variant thereof; or one copy of FAR1 and one copy of FAR16; or two copies of FAR1, one copy of FAR16 and one copy of FAR19.

### Desaturases and FARs

Any of the above FARs can be expressed together with any desaturase, in particular any of the desaturases described herein.

In some embodiments, the cell expresses:
a Plodia interpunctella Δ12 desaturase, for example Pid12 as set forth in SEQ ID NO: 2, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16; and/or
a FAR selected from: an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73;
   or functional variants thereof having at least 60% identity or similarity thereto.

In some embodiments, the cell expresses:
a Cadra cautella Δ12 desaturase, for example EcauDes12 as set forth in SEQ ID NO: 55, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16; and/or
a FAR selected from: an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73;
   or functional variants thereof having at least 60% identity or similarity thereto.

In some embodiments, the cell expresses:
an Ephestia kuehniella Δ12 desaturase, for example Eku_d12 as set forth in SEQ ID NO: 85, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16; and/or
a FAR selected from: an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73;
   or functional variants thereof having at least 60% identity or similarity thereto.

In some embodiments, the cell expresses:
an Ephestia elutella Δ12 desaturase, for example Ee_d12 as set forth in SEQ ID NO: 87, and
a further desaturase selected from: a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16; and/or
a FAR selected from: an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73;
   or functional variants thereof having at least 60% identity or similarity thereto.

It will be understood that a functional variant of a FAR or a desaturase having at least 60% identity or similarity to a given FAR or desaturase as above may have at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity or similarity to the FAR or the desaturase.

The yeast cell expressing said Δ12 desaturase, further desaturase, and FAR is capable of producing (Z9, E12)-tetradecadien-1-ol.

### Yeast Cell

The present invention provides a yeast cell which has been modified or engineered to produce desaturated compounds, in particular desaturated fatty acyl-CoAs and desaturated fatty alcohols having at least one double bond at position 12. Some of these are components of pheromones. The yeast cell disclosed herein thus provides an improved platform for environment-friendly pheromone production.

Accordingly, provided herein is a yeast cell capable of producing a desaturated fatty acyl-CoA having n′ double bond(s), wherein at least one of said double bond(s) is at position 12,

wherein said yeast cell expresses a heterologous Δ12 desaturase, said desaturase being capable of introducing a double bond at position 12 in a saturated or desaturated fatty acyl-CoA, preferably in a desaturated fatty acyl-CoA, having a carbon chain length of at least 13 and having n double bond(s),
wherein n and n′ are integers,
wherein 0≤n≤3 and wherein 1≤n′≤4.

In preferred embodiments, the saturated or desaturated fatty acyl-CoA used as a substrate and the desaturated fatty acyl-CoA having n′ double bonds have the same carbon chain length.

The heterologous Δ12 desaturase may be as disclosed herein in the section “Δ12 desaturase”. For example, the Δ12 desaturase may be a Plodia Δ12 desaturase, such as a Plodia interpunctella Δ12 desaturase, such as the Δ12 desaturase as set forth in SEQ ID NO: 2 (Pid12), or a functional variant thereof having at least 60% similarity or identity thereto. For example, the Δ12 desaturase may be a Cadra Δ12 desaturase, such as a Cadra cautella Δ12 desaturase, such as the Δ12 desaturase as set forth in SEQ ID NO: 55 (EcauDes12), or a functional variant thereof having at least 60% similarity or identity thereto. For example, the Δ12 desaturase may be an Ephestia Δ12 desaturase, such as an Ephestia kuehniella Δ12 desaturase or an Ephestia elutella Δ12 desaturase, such as the Δ12 desaturase as set forth in SEQ ID NO: 85 (Eku_d12) or SEQ ID NO: 87 (Ee_d12), or a functional variant thereof having at least 60% similarity or identity thereto.

In one embodiment, the yeast cell disclosed herein expresses a further desaturase that may be as disclosed herein in the section “Further desaturase”. For example, the further desaturase may be a desaturase capable of introducing a double bond in a saturated or desaturated fatty acid at a position other than position 12. In some embodiments, the desaturase is a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16, or functional variants thereof having at least 60% similarity or identity thereto.

The yeast cell expressing a heterologous Δ12 desaturase and a further desaturase is capable of producing a double desaturated fatty acyl-CoA with a double bond at position 12 and a double bond at one other position. In one embodiment, the yeast cell expressing a heterologous Δ12 desaturase and a further heterologous desaturase is capable of producing a fatty acyl-CoA with a double bond at position 12 and at position 9, such as for example (Z9, E12)-tetradecadienoyl-CoA.

In one embodiment, the yeast cell disclosed herein further expresses a FAR that may be as disclosed herein in the section “Fatty acyl-CoA reductase”. For example, the FAR may be an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73; or functional variants thereof having at least 60% similarity or identity thereto.

The yeast cell expressing a heterologous Δ12 desaturase, a further desaturase, and a FAR is capable of producing double desaturated fatty alcohols, such as for example (Z9, E12)-tetradecadien-1-ol. In other words, the yeast cell expressing a heterologous Δ12 desaturase, a further desaturase, and a FAR is capable of converting at least a part of the (Z9, E12)-tetradecadienoyl-CoA produced by the heterologous Δ12 desaturase and the further heterologous desaturase into (Z9, E12)-tetradecadien-1-ol by the action of the FAR.

In some embodiments, the genes encoding the heterologous Δ12 desaturase, the further heterologous desaturase or the FAR have been codon optimized for said yeast cell.

In one embodiment, at least one of the genes encoding the heterologous Δ12 desaturase, the further heterologous desaturase or the FAR is present in a high copy number in the yeast cell.

In one embodiment, at least one of the genes encoding the heterologous Δ12 desaturase, the further heterologous desaturase or the FAR is under the control of an inducible promoter.

In one embodiment, at least one of the genes encoding the heterologous Δ12 desaturase, the further heterologous desaturase or the FAR are each independently comprised within the genome of the cell or within a vector comprised within the yeast cell.

In one embodiment, the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi. Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.

In some embodiments, the yeast cell comprises a nucleic acid or a system of nucleic acids, as described in the section “Nucleic acid”.

The yeast cell according to the present invention may be comprised in a fermentation broth, a fermentation system, and/or a catalytic system. In other words, a fermentation broth, a fermentation system and/or a catalytic system may comprise the yeast cell according to the present invention.

In one embodiment, the yeast cell further expresses a heterologous NAD(P)H cytochrome b5 oxidoreductase (Ncb5or). The term ‘NAD(P)H cytochrome b5 oxidoreductase’ and ‘Ncb5or’ will be used herein interchangeably. The term ‘heterologous Ncb5or’ refers to an Ncb5or which is not naturally expressed by the organism, such as by the cell.

Ncb5or is an oxidoreductase acting on NADH or NADPH, with a heme protein as acceptor. It contains functional domains similar to cytochrome b5, cytochrome b5 reductase and CHORD-SGT1 (Deng, et al., 2010). Ncb5ors catalyze the reaction:

$$2 {Fe}^{3 +} + NAD ( P ) H <= > 2 {Fe}^{2 +} + H ++ NAD ( P ) +$$

Ncb5ors capable of catalyzing such reaction have EC number 1.6.2.2.

Expression of one or more Ncb5ors in a cell expressing a desaturase and/or a FAR has a positive effect on the activity of the desaturase and/or FAR, as it results in an increase in titer of desaturated fatty acyl-CoA and/or desaturated fatty alcohol. Thus, a yeast cell expressing a desaturase and/or a FAR and one or more Ncb5ors is capable of producing said compounds with a higher titer compared to a yeast cell expressing the same desaturase and/or FAR but no heterologous Ncb5or when cultivated in the same conditions.

The Ncb5or may be native to a plant, an insect or a mammal, such as Homo sapiens. In one embodiment, the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa. In one embodiment, the Ncb5or is native to an insect selected from Amyelois transitella, Agrotis segetum, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.

In one embodiment, the Ncb5or is selected from the group of Ncb5ors set forth in the table “Ncb5ors” in the section “Sequence overview” in patent application PCT/EP2022/062641 entitled “Improved methods and cells for increasing enzyme activity and production of insect pheromones”, filed by the same applicant on 10 May 2022.

Thus, in one embodiment, the Ncb5or is selected from the group of Ncb5ors set forth in SEQ ID NO: 37, SEQ ID NO: 38, SEQ ID NO: 39, SEQ ID NO: 40, SEQ ID NO: 41, SEQ ID NO: 88, SEQ ID NO: 89 and SEQ ID NO: 90 or functional variants having at least 70% identity or similarity thereto, such as at least 75% identity, such as at least 80% identity, such as at least 85% identity, such as at least 90% identity, such as at least 95% identity or similarity thereto.

In some embodiments, the yeast cell has reduced activity of one or proteins as disclosed in WO 2018/109163 and in European patent 3555268, in particular in the section entitled “Reduced activity of Hfd1, Hfd4, Pex10, Fao1, GPAT or a homologue thereof”. For example, the yeast cell may have a mutation resulting in reduced activity (i.e. downregulation) of Pex10, Hfd1, Hfd4, Fao1 and/or GPAT. Preferably, the yeast cell has at least one mutation resulting in reduced activity of at least Fao1 and one or more of Hfd1, Hfd4, Pex10 and/or GPAT. Such mutations may increase the production of desaturated fatty alcohol and/or desaturated fatty alcohol acetate in a yeast cell expressing a heterologous desaturase and a heterologous fatty acyl-CoA reductase.

Thus, in one embodiment, the yeast cell has a mutation leading to partial or total loss of activity of one or more of Hfd1, Hfd4, GPAT, Fao1, Pex10, such as having at least a mutation leading to partial or total loss of activity of Fao1 and one or more of Hfd1, Hfd4, Pex10 or GPAT.

In one embodiment, the yeast cell has a mutation in PEX10 (accession number: XP_501311.1) and at least one of HFD1 (accession number: XP_505802.1), HFD4 (accession number: XP_500380.1X), FAO1 (accession number: XP_500864.1) and/or GPAT (accession number: XP_501275.1), or a homologue thereof having at least 60% identity thereto.

In one embodiment, the yeast cell has a mutation in FAO1 (accession number: XP_500864.1) and at least one of HFD1 (accession number: XP_505802.1), HFD4 (accession number: XP_500380.1X), PEX10 (accession number: XP_501311.1) and/or GPAT (accession number: XP_501275.1), or a homologue thereof having at least 60% identity thereto.

In one embodiment, HFD1 (accession number: XP_505802.1), HFD4 (accession number: XP_500380.1X), PEX10 (accession number: XP_501311.1) and/or FAO1 (accession number: XP_500864.1) or homologue thereof having at least 60% identify thereto is deleted or mutated, resulting in partial loss of total loss of activity of Hfd1, Hfd4, Pex10, and/or Fao1, and/or GPAT or a homologue thereof having at least 60% identity thereto is mutated, resulting in reduced activity of GPAT.

In one embodiment, the yeast cell comprises a mutation in at least one POX gene, such as a POX gene selected from the group consisting of POX1 (accession number: 074934.1), POX2 (accession number: XP_505264.1), POX3 (accession number: XP_503244.1), POX4 (accession number: XP_504475.1), POX5 (accession number: XP_502199.1), and POX6 (accession number: XP_503632.1).

In one embodiment, the yeast cell comprises a deletion or mutation in POX1 (accession number: 074934.1), POX2 (accession number: XP_505264.1), POX3 (accession number: XP_503244.1), POX4 (accession number: XP_504475.1), POX5 (accession number: XP_502199.1), and/or POX6 (accession number: XP_503632.1) or a homologue thereof having at least 60% identify thereto, resulting in partial loss of total loss of activity of Pox1, Pox2, Pox3, Pox4, Pox5 and/or Pox6.

The yeast cell may be further engineered to express any type of protein. Expression of such other proteins may improve the production, such as the titer, of the compounds disclosed herein. In one embodiment, the yeast cell expresses:
i) a heterologous cytochrome b5;
ii) a heterologous cytochrome b5 reductase;
iii) a hemoglobin;
iv) a heterologous thioesterase gene; and/or
v) of a fusion protein of a fatty acyl synthase and of a thioesterase.

The native genes of the yeast cell may also be engineered. In one embodiment, the yeast cell has an inactivation or modification of:
i) native elongase(s) resulting in total or partial loss of activity;
ii) thioesterases(s) resulting in total or partial loss of activity; and/or
iii) activity of native fatty aldehyde dehydrogenase(s), fatty alcohol oxidase(s), peroxisome biogenesis factor and/or fatty acyl synthase(s).

In some embodiments, the yeast cell is further modified so that the availability of fatty acyls having a chain length of 14 is increased or further increased. For instance, the fatty acid synthase complex may be engineered so that formation of tetradecanoyl-CoA is increased. The fatty acid synthase complex (EC 2.3.1.86) consists of two subunits, Fas1 (beta subunit) and Fas2 (alpha subunit). The alpha subunit comprises a ketoacyl synthase domain (a “binding pocket”) which is hypothesized to be involved in determining the length of the synthesized fatty acids.

Accordingly, in order to direct the metabolic flux towards production of desaturated fatty alcohols, acetates or aldehydes having a chain length of 14 carbons (C14), the yeast cell may further express a fatty acyl synthase variant having a modified ketone synthase domain. Without being bound by theory, it is hypothesized that the modified ketone synthase domain results in a modified binding pocket, which thus more readily accommodates medium length substrates such as C14 substrates, thereby producing a higher proportion of C14 products.

In one embodiment, the yeast cell is a yeast cell as described herein, wherein the cell further expresses a modified fatty acid synthase complex. In one embodiment, the fatty acid synthase complex is modified by mutating the gene encoding the alpha subunit of the complex. In some embodiments, the mutation is in the gene encoding FAS2. In one embodiment, the yeast cell is a Yarrowia lipolytica yeast cell and the mutation may result in modification of one or more of residue 1220 (I1220), residue 1217 (M1217) or residue 1226 (M1226) of Yarrowia lipolytica FAS2, resulting in a variant FAS2. The skilled person will know how to design such mutations.

Preferably, the mutation results in an I1220F variant, an I1220W variant, an I1220Y variant or an I1220H variant. In a specific embodiment, the mutation results in an I1220F variant. In some embodiments, the mutation results in an M1217F variant, an M1217W variant, an M1217Y variant or an M1217H variant. In other embodiments, the mutation results in an M1226F variant, an M1226W variant, an M1226Y variant or an M1226H variant. Yeast cells with more than one of the above mutations are also contemplated, such as two mutations or three mutations at residue I1220, M1217 or M1226.

The fatty acyl-CoAs produced by the cell may be further converted by the cell to the corresponding fatty acids. Said fatty acids may be free fatty acids or part of triacylglycerides (lipids).

In one embodiment, the yeast cell is capable of producing an E12-fatty acid and/or (Z9, E12)-tetradecadienoic acid with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the yeast cell is capable of producing an E12-fatty alcohol and/or (Z9, E12)-tetradecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

The yeast cell disclosed herein expressing a heterologous Δ12 desaturase, optionally a further heterologous desaturase, and further optionally a heterologous FAR is capable of producing desaturated fatty acyl-CoAs and/or desaturated fatty alcohols with a carbon chain length of at least 13 having a double bond at position 12 and optionally other double bond(s) at position(s) other than position 12.

Thus, provided herein are desaturated fatty acyl-CoAs and desaturated fatty alcohols obtainable according to the methods presented herein.

Also provided herein is a yeast cell capable of producing a pheromone compound according the invention, wherein the yeast cell expresses a heterologous Δ12 fatty acyl-CoA desaturase.

In some embodiments, the yeast cell expresses a further heterologous desaturase.

In some embodiments, the yeast cell further expresses at least one alcohol-forming fatty acyl-CoA reductase (FAR).

Further provided herein is the use of a desaturated fatty acyl-CoA and/or a desaturated fatty alcohol obtainable according to the methods presented herein. Such compounds can be used for example for monitoring the presence of pest or disrupting the presence of pest; for example they can be formulated as a pheromone composition.

### Desaturated Fatty Acyl-CoAs

In one embodiment, the yeast cell expresses a heterologous Δ12 desaturase, and optionally a further heterologous desaturase, said yeast cell being capable of producing a desaturated fatty acyl-CoA having a double bond at position 12, and optionally a double bond at a position other than position 12.

Thus, the yeast cell disclosed herein is capable of producing a desaturated fatty acyl-CoA having n′ double bond(s), wherein at least one of said double bond(s) is at position 12,
wherein said yeast cell expresses a heterologous Δ12 desaturase, said desaturase being capable of introducing a double bond at position 12 in a saturated or desaturated fatty acyl-CoA, preferably a desaturated fatty acyl-COA, having a carbon chain length of at least 13 and having n double bond(s),
wherein n and n′ are integers,
wherein 0≤n≤3 and wherein 1≤n′≤4.

In preferred embodiments, the saturated or desaturated fatty acyl-CoA used as a substrate and the desaturated fatty acyl-CoA having n′ double bonds have the same carbon chain length.

In one embodiment the yeast cell expresses a further desaturase as disclosed herein in the section “Further desaturase”.

In one embodiment, n=0. In other words, the yeast cell is capable of introducing up to 4 double bonds, such as 1, 2, 3, or 4 double bonds, into a saturated fatty acyl-CoA having no double bonds. Thus, said yeast cell is capable of producing a desaturated fatty acyl-CoA having 1, 2, 3, or 4 double bonds. In a preferred embodiment, the yeast cell is capable of introducing a double bond into tetradecanoyl-CoA, thereby converting said tetradecanoyl-CoA to (E12)-tetradecenoyl-CoA.

In one embodiment, n=1. In other words, the yeast is capable of introducing up to 3 double bonds, such as 1, 2 or 3 double bonds into a desaturated fatty acyl-CoA having one double bond at a position other than position 12, such as at position 8, 9, 10, 11, 13, 14, 15, 16, 17, 18, 19, 20, 21 or 22. Thus, said yeast cell is capable of producing a desaturated fatty acyl-CoA having 2, 3 or 4 double bonds, preferably a desaturated fatty acyl-CoA having 2 double bonds. In one embodiment, the yeast cell is capable of introducing a double bond into a desaturated fatty acyl-CoA having a double bond at position 9. In a preferred embodiment, the yeast cell is capable of introducing a double bond into (Z9)-tetradecenoyl-CoA, thereby converting said (Z9)-tetradecenoyl-CoA to (Z9, E12)-tetradecadienoyl-CoA.

In one embodiment, n=2. In other words, the yeast cell is capable of introducing up to 2 double bonds, such as 1 or 2 double bonds, into a desaturated fatty acyl-CoA having two double bonds at a position other than position 12, such as a double bond in at least two of positions 8, 9, 10, 11, 13, 14, 15, 16, 17, 18, 19, 20, 21 or 22. Thus, said yeast cell is capable of producing a desaturated fatty acyl-CoA having 3 or 4 double bonds.

In one embodiment, n=3. In other words, the yeast is capable of introducing one double bond into a desaturated fatty acyl-CoA having three double bonds at a position other than position 12, such as one double bond at least three of positions 8, 9, 10, 11, 13, 14, 15, 16, 17, 18, 19, 20, 21 or 22. Thus, said yeast cell is capable of producing a desaturated fatty acyl-CoA having 4 double bonds.

In one embodiment, n′=1 and n=0. In other words, the yeast cell is capable of converting a saturated fatty acyl-CoA with no double bond into a desaturated fatty acyl-CoA with one double bond at position 12.

In one embodiment, n′=2 and n=0. In other words, the yeast cell is capable of converting a saturated fatty acyl-CoA into a desaturated fatty acyl-CoA with two double bonds, wherein one double bond is at position 12.

In one embodiment, n′=3 and n=0. In other words, the yeast cell is capable of converting a saturated fatty acyl-CoA into a desaturated fatty acyl-CoA with three double bonds, wherein one double bond is at position 12.

In one embodiment, n′=4 and n=0. In other words, the yeast cell is capable of converting a saturated fatty acyl-CoA into a desaturated fatty acyl-CoA with four double bonds, wherein one double bond is at position 12.

In a preferred embodiment, n′=2 and n=1. In other words, the yeast cell is capable of converting a desaturated fatty acyl-CoA with one double bond into a desaturated fatty acyl-CoA with two double bonds, wherein one double bond is at position 12.

In one embodiment, n′=3 and n=1. In other words, the yeast cell is capable of converting a desaturated fatty acyl-CoA with one double bond into a desaturated fatty acyl-CoA with three double bonds, wherein one double bond is at position 12.

In one embodiment, n′=4 and n=1. In other words, the yeast cell is capable of converting a desaturated fatty acyl-CoA with one double bond into a desaturated fatty acyl-CoA with four double bonds, wherein one double bond is at position 12.

In one embodiment, n′=3 and n=2. In other words, the yeast cell is capable of converting a desaturated fatty acyl-CoA with two double bonds into a desaturated fatty acyl-CoA with three double bonds, wherein one double bond is at position 12.

In one embodiment, n′=4 and n=2. In other words, the yeast cell is capable of converting a desaturated fatty acyl-CoA with two double bonds into a desaturated fatty acyl-CoA with four double bonds, wherein one double bond is at position 12.

In one embodiment, n′=4 and n=3. In other words, the yeast cell is capable of converting a desaturated fatty acyl-CoA with three double bonds into a desaturated fatty acyl-CoA with four double bonds, wherein one double bond is at position 12.

In one embodiment, the saturated or desaturated fatty acyl-CoA having a carbon chain length of at least 13 and having n double bonds, has a carbon chain length of at least 13, such as at least 14, such as at least 15, such as at least 16, such as at least 17, such as at least 18, such as at least 19, such as at least 20, such as at least 21, such as at least 22. In a preferred embodiment, the saturated or desaturated fatty acyl-CoA having a carbon chain length of at least 13 and having n double bonds, has a carbon chain length of 13, 14, 15, 16, 17 or 18.

In one embodiment, the saturated or desaturated fatty acyl-CoA having a carbon chain length of at least 13 and having n double bonds has a length of 14 and is tetradecanoyl-CoA or (Z9)-tetradecenoyl-CoA. In a preferred embodiment, the saturated or desaturated fatty acyl-CoA having a carbon chain length of at least 13 and having n double bonds has a length of 14 and is (Z9)-tetradecenoyl-CoA.

In one embodiment, the desaturated fatty acyl-CoA having n′ double bond(s) has a carbon chain length of at least 13, such as at least 14, such as at least 15, such as at least 16, such as at least 17, such as at least 18, such as at least 19, such as at least 20, such as at least 21, such as at least 22. In one embodiment, the desaturated fatty acyl-CoA having n′ double bond(s) has a carbon chain length of at most 18, such as at most 17, such as at most 16, such as at most 15, such as at most 14, such as at most 13, such as at most 12. In a preferred embodiment, the desaturated fatty acyl-CoA having n′ double bond(s) has a carbon chain length of 13, 14, 15, 16, 17 or 18.

In one embodiment, the desaturated fatty acyl-CoA having n′ double bond(s) has one double bond, and is (E12)-tetradecenoyl-CoA.

In one embodiment, the desaturated fatty acyl-CoA having n′ double bond(s) has two double bonds, and is (Z9, E12)-tetradecadienoyl-CoA.

Thus, also provided herein is (E12)-tetradecenoyl-CoA and/or (Z9, E12)-tetradecadienoyl-CoA obtainable according to the methods presented herein.

### Desaturated Fatty Alcohols

In one embodiment, the yeast cell expressing a Δ12 desaturase, and optionally a further desaturase, also expresses a FAR as presented herein in the section “Fatty acyl-CoA reductase”. Said yeast cell may be capable of producing a desaturated fatty alcohol having a double bond at position 12, and optionally a double bond at a position other than position 12, such as at position 9.

Said FAR, which is necessary for producing the desaturated fatty alcohol, can be any FAR described herein in the section “Fatty acyl-CoA reductase”, and said desaturated fatty alcohol can be any desaturated fatty alcohol corresponding to the fatty acyl-CoAs described herein in the section “Desaturated fatty acyl-CoAs”.

In one embodiment, the desaturated fatty alcohol is an (E12)-fatty alcohol, such as (E12)-tetradecen-1-ol. In one embodiment, the desaturated fatty alcohol is (Z9, E12)-tetradecadien-1-ol.

Thus, further provided herein is an (E12)-fatty alcohol, such as (E12)-tetradecen-1-ol, and/or (Z9, E12)-tetradecadien-1-ol obtainable by the methods presented herein.

### Method for Production of a Desaturated Fatty Acyl-CoA

Provided herein is a method for producing a desaturated fatty acyl-CoA having n′ double bond(s), wherein at least one of said double bond(s) is at position 12 in a yeast cell, said method comprising the steps of:
i) providing a yeast cell as defined herein,
ii) incubating said yeast cell in a medium under conditions allowing expression of a heterologous Δ12 desaturase, said desaturase being capable of introducing a double bond at position 12 in a saturated or desaturated fatty acyl-CoA, preferably a desaturated fatty acyl-CoA, having a carbon chain length of at least 13 and having n double bonds,
   wherein n and n′ are integers,
   wherein 0≤n≤3 and wherein 1≤n′≤4,
   thereby producing a desaturated fatty acyl-CoA having n′ double bond(s), wherein at least one of said double bond(s) is at position 12.

In preferred embodiments, the saturated or desaturated fatty acyl-CoA used as a substrate and the desaturated fatty acyl-CoA having n′ double bonds have the same carbon chain length.

In one embodiment, the heterologous Δ12 desaturase is as defined herein in the section “Δ12 desaturase”. For example, the Δ12 desaturase may be a Plodia Δ12 desaturase, such as a Plodia interpunctella Δ12 desaturase, such as the Δ12 desaturase as set forth in SEQ ID NO: 2 (Pid12) or a functional variant thereof having at least 60% identity or similarity thereto. For example, the Δ12 desaturase may be a Cadra Δ12 desaturase, such as a Cadra cautella Δ12 desaturase, such as the Δ12 desaturase as set forth in SEQ ID NO: 55 (EcauDes12), or a functional variant thereof having at least 60% similarity or identity thereto. For example, the Δ12 desaturase may be an Ephestia Δ12 desaturase, such as an Ephestia kuehniella Δ12 desaturase or an Ephestia elutella Δ12 desaturase, such as the Δ12 desaturase as set forth in SEQ ID NO: 85 (Eku_d12) or SEQ ID NO: 87 (Ee_d12), or a functional variant thereof having at least 60% similarity or identity thereto.

In one embodiment, the further heterologous desaturase is as defined herein in the section “Further desaturase”. For example, the further desaturase may be a desaturase capable of introducing a double bond in a saturated or desaturated fatty acid at a position other than position 12. In some embodiments, the further desaturase is a desaturase native to the yeast cell, such as a homologous desaturase. In some embodiments, the further desaturase is a desaturase which is not native to the yeast cell, such as a heterologous desaturase. In some embodiments, the desaturase is a Drosophila desaturase, such as a Drosophila virilis desaturase, for example Desat61 as set forth in SEQ ID NO: 4, or such as a Drosophila melanogaster desaturase, for example Desat24 as set forth in SEQ ID NO: 6, or such as a Drosophila grimshawi desaturase, for example Desat59 as set forth in SEQ ID NO: 8, or such as a Drosophila yakuba desaturase, for example Desat56 as set forth in SEQ ID NO: 57, or such as a Drosophila ananassae desaturase, for example Desat60 as set forth in SEQ ID NO: 59; an Amyelois desaturase, such as an Amyelois transitella desaturase, for example Desat17 as set forth in SEQ ID NO: 10 or Desat18 as set forth in SEQ ID NO: 12; a Choristoneura desaturase, such as a Choristoneura parallela desaturase, for example Desat74 as set forth in SEQ ID NO: 61; a Spodoptera desaturase, such as a Spodoptera litura desaturase, for example Desat26 as set forth in SEQ ID NO: 18; a Saccharomyces desaturase, such as a Saccharomyces cerevisiae desaturase, for example Desat42 as set forth in SEQ ID NO: 14; a Yarrowia desaturase, such as a Yarrowia lipolytica desaturase, for example Desat69 as set forth in SEQ ID NO: 16, or functional variants thereof having at least 60% similarity or identity thereto.

The saturated or desaturated fatty acyl-CoA having a carbon chain length of at least 13 and having n double bond(s) may be as defined herein in the section “Desaturated fatty acyl-CoA”. In one embodiment, the saturated or desaturated fatty acyl-CoA having a carbon chain length of at least 13 and having n double bonds, is tetradecanoyl-CoA or (Z9)-tetradecenoyl-CoA.

The desaturated fatty acyl-CoA having n′ double bond(s) may be as defined herein in the section “Desaturated fatty acyl-CoA”. In one embodiment, the desaturated fatty acyl-CoA having n′ double bond(s) is (E12)-tetradecenoyl-CoA and/or (Z9, E12)-tetradecadienoyl-CoA.

In one embodiment, the method yields a desaturated fatty acyl-CoA having n′ double bond(s), such as (E12)-tetradecenoyl-CoA and/or (Z9, E12)-tetradecadienoyl-CoA, at a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the yeast cell further expresses at least one alcohol-forming fatty acyl-CoA reductase (FAR) capable of converting at least a part of (E12)-tetradecenoyl-CoA and/or (Z9, E12)-tetradecadienoyl-CoA into (E12)-tetradecen-1-ol and/or (Z9, E12)-tetradecadien-1-ol, respectively.

The FAR may be as defined herein in the section “Fatty acyl-CoA reductase”. For example, the FAR may be an Agrotis FAR, such as an Agrotis segetum FAR, for example FAR12 as set forth in SEQ ID NO: 75; a Bicyclus FAR, such as a Bicyclus anynana FAR, for example FAR11 as set forth in SEQ ID NO: 77; a Helicoverpa FAR, such as a Helicoverpa armigera FAR or a Helicoverpa assulta FAR, for example FAR1 as set forth in SEQ ID NO: 20 or FAR6 as set forth in SEQ ID NO: 71; a Heliothis FAR, such as a Heliothis subflexa FAR or a Heliothis virescens FAR, for example FAR4 as set forth in SEQ ID NO: 69 or FAR5 as set forth in SEQ ID NO: 79; an Ostrinia FAR, such as an Ostrinia furnacalis FAR, an Ostrinia zag FAR or an Ostrinia zea FAR, for example FAR44 as set forth in SEQ ID NO: 63, FAR48 as set forth in SEQ ID NO: 65 or FAR49 as set forth in SEQ ID NO: 67; a Plodia FAR, such as a Plodia interpunctella FAR, for example FAR28 as set forth in SEQ ID NO: 28 or FAR32 as set forth in SEQ ID NO: 30; a Spodoptera FAR, such as a Spodoptera exigua FAR, for example FAR16 as set forth in SEQ ID NO: 22, or FAR17 as set forth in SEQ ID NO: 24, or such as a Spodoptera litura FAR, for example FAR19 as set forth in SEQ ID NO: 26; a Trichoplusia FAR, such as a Trichoplusia ni FAR, for example FAR38 as set forth in SEQ ID NO: 32; an Yponomeuta FAR, such as an Yponomeuta rorellus FAR, for example FAR8 as set forth in SEQ ID NO: 73; or functional variants thereof having at least 60% similarity or identity thereto.

In one embodiment, the method yields (Z9, E12)-tetradecadien-1-ol at a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 10 mg/L, such as at least 15 mg/L, such as at least 20 mg/L, such as at least 25 mg/L, such as at least 30 mg/L, such as at least 35 mg/L, such as at least 50 mg/L, such as at least 60 mg/L, such as at least 70 mg/L, such as at least 80 mg/L, such as at least 90 mg/L, such as at least 100 mg/L, such as at least 200 mg/L, such as at least 300 mg/L, such as at least 400 mg/L, such as at least 500 mg/L, such as at least 750 mg/L, such as at least 1 g/L, such as at least 2 g/L, such as at least 5 g/L, such as at least 10 g/L, such as at least 20 g/L, such as at least 50 g/L, such as at least 100 g/L, or more.

In one embodiment, the method further comprises the step of recovering the E12-fatty alcohol and/or the (Z9, E12)-tetradecadien-1-ol.

In one embodiment, the E12-fatty alcohol and/or the (Z9, E12)-tetradecadien-1-ol is further modified into an E12-fatty alcohol acetate and/or (Z9, E12)-tetradecadien-1-ol acetate, respectively.

In one embodiment, the conversion of the E12-fatty alcohol and/or the (Z9, E12)-tetradecadien-1-ol to an E12-fatty alcohol acetate and/or (Z9, E12)-tetradecadien-1-ol acetate is performed in vitro.

In one embodiment the conversion of the E12-fatty alcohol and/or the (Z9, E12)-tetradecadien-1-ol to an E12-fatty alcohol acetate and/or (Z9, E12)-tetradecadien-1-ol acetate is performed in vivo by further expressing in the cell an acetyltransferase capable of converting the E12-fatty alcohol and/or the (Z9, E12)-tetradecadien-1-ol to an E12-fatty alcohol acetate and/or (Z9, E12)-tetradecadien-1-ol acetate, respectively.

In one embodiment the method further comprises a step of converting the E12-fatty alcohol and/or the (Z9, E12)-tetradecadien-1-ol to an (E12)-fatty aldehyde and/or (Z9, E12)-tetradecadienal. In one embodiment, the conversion to an aldehyde is a chemical or an enzymatic conversion.

Compounds of the present disclosure may be oxidized as described herein, by methods known to the skilled person or as described in European patent application EP21190097.2 filed on 6 Aug. 2021 by same applicant. In one embodiment, the E12-fatty alcohol and/or the (Z9, E12)-tetradecadien-1-ol is chemically oxidized into an (E12)-fatty aldehyde and/or (Z9, E12)-tetradecadienal.

Compounds of the present disclosure may be acetylated as described herein or by methods known to the skilled person. In one embodiment, the E12-fatty alcohol and/or the (Z9, E12)-tetradecadien-1-ol is chemically acetylated into an E12-fatty alcohol acetate and/or (Z9, E12)-tetradecadien-1-ol acetate.

In one embodiment, the method further comprises the step of formulating the E12-fatty alcohol, such as the (E12)-tetradecen-1-ol, the (Z9, E12)-tetradecadien-1-ol, the E12-fatty alcohol acetate, the (Z9, E12)-tetradecadien-1-ol acetate, the (E12)-fatty aldehyde and/or the (Z9, E12)-tetradecadienal in a pheromone composition as defined herein in the section “Pheromone composition”.

Thus, provided herein is a method for producing (Z9, E12)-tetradecadien-1-ol, said method comprising the steps of:
i) providing a yeast cell as disclosed herein,
ii) incubating said yeast cell in a medium under conditions allowing expression of:
   a. a further desaturase, said desaturase being capable of introducing a double bond at position 9 in tetradecanoyl-CoA, thereby converting at least part of tetradecanoyl-CoA to (Z9)-tetradecenoyl-CoA;
   b. a heterologous Δ12 desaturase, said desaturase being capable of introducing a double bond at position 12 in (Z9)-tetradecenoyl-CoA, thereby converting at least part of (Z9)-tetradecenoyl-CoA to (Z9, E12)-tetradecadienoyl-CoA;
   c. a FAR, said FAR being capable of converting at least part of (Z9, E12)-tetradecadienoyl-CoA into (Z9, E12)-tetradecadien-1-ol;
iii) optionally, recovering the (Z9, E12)-tetradecadien-1-ol;
iv) optionally, converting the (Z9, E12)-tetradecadien-1-ol into (Z9, E12)-tetradecadien-1-ol acetate;
v) optionally, converting the (Z9, E12)-tetradecadien-1-ol into (Z9, E12)-tetradecadienal;
vi) optionally, formulating the (Z9, E12)-tetradecadien-1-ol, the (Z9, E12)-tetradecadien-1-ol acetate, and/or the (Z9, E12)-tetradecadienal into a pheromone composition.

### Nucleic Acid

Provided herein is a nucleic acid or a system of nucleic acids for modifying a yeast cell, said nucleic acid or system comprising at least one polynucleotide encoding a heterologous Δ12 desaturase, said desaturase being capable of introducing a double bond at position 12 in a saturated or desaturated fatty acyl-CoA having a carbon chain length of at least 13 and having n double bonds thereby producing a desaturated fatty acyl-CoA having n′ double bond(s), wherein at least one of said double bond(s) is at position 12,
wherein n and n′ are integers,
wherein 0≤n≤3 and wherein 1≤n′≤4.

In one embodiment, the heterologous Δ12 desaturase is encoded by a nucleic acid having at least 60% identity to the nucleic acid encoding a Plodia interpunctella Δ12 desaturase as set forth in SEQ ID NO: 1, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous Δ12 desaturase is encoded by a nucleic acid having at least 60% identity to the nucleic acid encoding a Cadra cautella Δ12 desaturase as set forth in SEQ ID NO: 54, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous Δ12 desaturase is encoded by a nucleic acid having at least 60% identity to the nucleic acid encoding an Ephestia kuehniella Δ12 desaturase as set forth in SEQ ID NO: 84, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous Δ12 desaturase is encoded by a nucleic acid having at least 60% identity to the nucleic acid encoding an Ephestia elutella Δ12 desaturase as set forth in SEQ ID NO: 86, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the nucleic acid or the system of nucleic acids further comprises a polynucleotide encoding a further heterologous desaturase capable of introducing a double bond at any position which is not position 12 in a saturated or desaturated fatty acyl-CoA having a carbon chain length of at least 13 and having n double bonds.

In some embodiments, the further heterologous desaturase is encoded by a nucleic acid having at least 60% identity to a nucleic acid selected from the group of desaturases set forth in SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 56, SEQ ID NO: 58 and SEQ ID NO: 60, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the further heterologous desaturase is encoded by a nucleic acid having at least 60% identity to the nucleic acid encoding a Drosophila virilis desaturase, as set forth in SEQ ID NO: 3.

In one embodiment, the further heterologous desaturase is encoded by a nucleic acid having at least 60% identity to the nucleic acid encoding a Drosophila melanogaster desaturase, as set forth in SEQ ID NO: 5.

In one embodiment, the further heterologous desaturase is encoded by a nucleic acid having at least 60% identity to the nucleic acid encoding a Drosophila grimshawi desaturase, as set forth in SEQ ID NO: 7.

In one embodiment, the further heterologous desaturase is encoded by a nucleic acid having at least 60% identity to the nucleic acid encoding a Amyelois transitella desaturase, as set forth in SEQ ID NO: 9 or SEQ ID NO: 11.

In one embodiment, the further heterologous desaturase is encoded by a nucleic acid having at least 60% identity to the nucleic acid encoding a Saccharomyces cerevisiae desaturase, as set forth in SEQ ID NO: 13.

In one embodiment, the further heterologous desaturase is encoded by a nucleic acid having at least 60% identity to the nucleic acid encoding a Yarrowia lipolytica desaturase, as set forth in SEQ ID NO: 15.

In one embodiment, the further heterologous desaturase is encoded by a nucleic acid having at least 60% identity to the nucleic acid encoding a Spodoptera litura desaturase, as set forth in SEQ ID NO: 17.

In one embodiment, the further heterologous desaturase is encoded by a nucleic acid having at least 60% identity to the nucleic acid encoding a Drosophila yakuba desaturase, as set forth in SEQ ID NO: 56.

In one embodiment, the further heterologous desaturase is encoded by a nucleic acid having at least 60% identity to the nucleic acid encoding a Drosophila ananassae desaturase, as set forth in SEQ ID NO: 58.

In one embodiment, the further heterologous desaturase is encoded by a nucleic acid having at least 60% identity to the nucleic acid encoding a Choristoneura parallela desaturase, as set forth in SEQ ID NO: 60.

In one embodiment, the nucleic acid or system of nucleic acids further comprises a polynucleotide encoding a FAR capable of converting at least a part of the double desaturated (Z9, E12)-tetradecadienoyl-CoA into (Z9, E12)-tetradecadien-1-ol.

In some embodiments, the FAR is encoded by a nucleic acid having at least 60% identity to a nucleic acid selected from the group of FARs set forth in SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76 and SEQ ID NO: 78, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the FAR is encoded by a nucleic acid having at least 60% identity to the nucleic acid encoding an Agrotis segetum FAR, as set forth in SEQ ID NO: 74.

In one embodiment, the FAR is encoded by a nucleic acid having at least 60% identity to the nucleic acid encoding a Bicyclus anynana FAR, as set forth in SEQ ID NO: 76.

In one embodiment, the FAR is encoded by a nucleic acid having at least 60% identity to the nucleic acid encoding a Helicoverpa armigera FAR, as set forth in SEQ ID NO: 19.

In one embodiment, the FAR is encoded by a nucleic acid having at least 60% identity to the nucleic acid encoding a Helicoverpa assulta FAR, as set forth in SEQ ID NO: 70.

In one embodiment, the FAR is encoded by a nucleic acid having at least 60% identity to the nucleic acid encoding a Heliothis subflexa FAR, as set forth in SEQ ID NO: 68.

In one embodiment, the FAR is encoded by a nucleic acid having at least 60% identity to the nucleic acid encoding a Heliothis virescens FAR, as set forth in SEQ ID NO: 78.

In one embodiment, the FAR is encoded by a nucleic acid having at least 60% identity to the nucleic acid encoding an Ostrinia furnacalis FAR, as set forth in SEQ ID NO: 62.

In one embodiment, the FAR is encoded by a nucleic acid having at least 60% identity to the nucleic acid encoding an Ostrinia zag FAR, as set forth in SEQ ID NO: 64.

In one embodiment, the FAR is encoded by a nucleic acid having at least 60% identity to the nucleic acid encoding an Ostrinia zea FAR, as set forth in SEQ ID NO: 66.

In one embodiment, the FAR is encoded by a nucleic acid having at least 60% identity to the nucleic acid encoding a Plodia interpunctella FAR, as set forth in SEQ ID NO: 27 or SEQ ID NO: 29.

In one embodiment, the FAR is encoded by a nucleic acid having at least 60% identity to the nucleic acid encoding a Spodoptera exigua FAR, as set forth in SEQ ID NO: 21 or SEQ ID NO: 23.

In one embodiment, the FAR is encoded by a nucleic acid having at least 60% identity to the nucleic acid encoding a Spodoptera litura FAR, as set forth in SEQ ID NO: 25.

In one embodiment, the FAR is encoded by a nucleic acid having at least 60% identity to the nucleic acid encoding a Trichoplusia ni FAR, as set forth in SEQ ID NO: 31.

In one embodiment, the FAR is encoded by a nucleic acid having at least 60% identity to the nucleic acid encoding an Yponomeuta rorellus FAR, as set forth in SEQ ID NO: 72.

Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to the given nucleic acid, or more.

The system of nucleic acids can thus comprise a plurality of polynucleotides encoding the enzymes that are to be expressed in the yeast cell, in particular a heterologous Δ12 desaturase as described herein, and optionally a further heterologous desaturase as described herein, and further optionally a FAR and/or additional enzymes such as an Ncb5or.

### Recovery

It may be desirable to recover the products obtained by the methods disclosed herein. Thus, the present methods may comprise a further step of recovering the desaturated fatty acids in free form or as lipids, the desaturated fatty alcohol, the desaturated fatty alcohol acetate, and/or the desaturated fatty aldehyde produced according to the methods presented herein.

In some embodiments, the method comprises a step of recovering the desaturated fatty alcohols. In other embodiments, the method comprises a step of recovering the desaturated fatty alcohol acetates.

Methods for recovering the products obtained by the present invention are known in the art and may comprise an extraction with a hydrophobic solvent such as decane, hexane or a vegetable oil.

The recovered products may be modified further, for example may the desaturated fatty alcohols be converted to the corresponding desaturated fatty aldehydes or fatty acetates as described herein above. In embodiments where desaturated fatty aldehydes and/or fatty acetates are directly produced in the culture medium, e.g. in vivo or by contacting the cells with the relevant enzymes, the desaturated fatty aldehydes and/or fatty acetates may also be recovered.

As described in application WO 2021/078452 filed by same applicant on 22 Sep. 2020 and entitled “Improved methods for production, recovery and secretion of hydrophobic compounds in a fermentation”, when a fermentation system is used to cultivate the cell, in particular a yeast cell, which is capable of producing desaturated fatty alcohols, desaturated fatty alcohol acetates and/or desaturated fatty aldehydes, the addition of an extractant to the culture medium may further increase titers and extracellular secretion. In some embodiments, the medium comprises an extractant in an amount equal to or greater than its cloud concentration in an aqueous solution, wherein the extractant a non-ionic surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone and ethoxylated and propoxylated C₁₆-C₁₈ alcohol-based antifoaming agents and combinations thereof. In some embodiments:
the non-ionic surfactant is an ethoxylated and propoxylated C₁₆-C₁₈ alcohol-based antifoaming agent, such as C₁₆-C₁₈ alkyl alcohol ethoxylate propoxylate (CAS number 68002-96-0), and wherein the culture medium comprises at least 1% vol/vol of C₁₆-C₁₈ alkyl alcohol ethoxylate propoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol C₁₆-C₁₈ alkyl alcohol ethoxylate propoxylate, or more,
the non-ionic surfactant is a polyethylene polypropylene glycol, for example Kollliphor® P407 (CAS number 9003-11-6), and wherein the culture medium comprises at least 10% vol/vol of polyethylene polypropylene glycol such as Kolliphor® P407, such as at least 11% vol/vol, such as at least 12% vol/vol, such as at least 13% vol/vol, such as at least 14% vol/vol, such as at least 15% vol/vol, such as at least 16% vol/vol, such as at least 17% vol/vol, such as at least 18% vol/vol, such as at least 19% vol/vol, such as at least 20% vol/vol, such as at least 25% vol/vol, such as at least 30% vol/vol, such as at least 35% vol/vol of polyethylene polypropylene glycol such as Kolliphor® P407, or more,
the non-ionic surfactant is a mixture of polyether dispersions, such as antifoam 204, and wherein the culture medium comprises at least 1% vol/vol of a mixture of polyether dispersions such as antifoam 204, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol of a mixture of polyether dispersions such as antifoam 204, or more; and/or
the non-ionic surfactant is a non-ionic surfactant comprising polyethylene glycol monostearate such as simethicone, and wherein the culture medium comprises at least 1% vol/vol of polyethylene glycol monostearate or simethicone, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol polyethylene glycol monostearate or simethicone, or more.

In other embodiments, the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.

The recovered products, i.e. the desaturated fatty alcohols, the desaturated fatty alcohol acetates, and/or the desaturated fatty aldehydes, may also be formulated into a pheromone composition, such as described in the section “Pheromone composition”. In one embodiment, said recovered products formulated into a pheromone composition are (Z9, E12)-tetradecadien-1-ol, (Z9, E12)-tetradecadien-1-ol acetate, and/or (Z9, E12)-tetradecadienal. The composition may further comprise one or more additional compounds such as a liquid or solid carrier or substrate. Fatty aldehydes obtained from said fatty alcohols may also be comprised in such compositions.

Fatty acids can be recovered by methods known in the art, e.g. after homogenisation of the leaves and recovery of the lipids by methods known in the art. The recovered lipids are hydrolysed into free fatty acids and esterified to fatty acid alkyl ester, followed by a reduction to either fatty alcohols or fatty aldehydes.

### Kit

Provided herein is a kit of parts for performing the present methods. The kit of parts may comprise a yeast cell “ready to use” as described herein. In one embodiment, the yeast cell is a Yarrowia cell, such as a Yarrowia lipolytica cell. In one embodiment, the yeast cell is a Saccharomyces cell, such as a Saccharomyces cerevisiae cell.

In one embodiment, the kit of parts comprises a nucleic acid or system of nucleic acids encoding the activities of interest to be introduced in the organism, such as the system of nucleic acids described in the section “Nucleic acid” herein. The nucleic acid or system of nucleic acids may be provided as a plurality of nucleic acid constructs, such as a plurality of vectors, wherein each vector encodes one or several of the desired activities.

The kit of parts may optionally comprise the yeast cell to be modified.

The kit of parts may also comprise instructions for use.

In some embodiments, the kit of parts comprises all or a combination of the above.

### Pheromone Composition

The present invention provides compounds, in particular desaturated fatty alcohols, and desaturated fatty alcohol acetates as well as derivatives thereof such as desaturated fatty aldehydes, and their use. In particular, the desaturated compounds obtainable using the present cells and methods are useful as components of pheromone compositions. Such pheromone compositions may be useful for integrated pest management. They can be used as is known in the art for e.g. mating disruption.

Thus, the desaturated fatty alcohols, the desaturated fatty alcohol acetates, and the desaturated fatty aldehydes obtainable by the present methods or using the present yeast cells, may be formulated in a pheromone composition. In one embodiment (E12)-fatty alcohol, (Z9, E12)-tetradecadien-1-ol and/or the corresponding fatty aldehydes and/or fatty acetates are formulated in a pheromone composition

Such pheromone compositions may be used as integrated pest management products, which can be used in a method of monitoring the presence of pest or in a method of disrupting the mating of pest.

Thus, provided herein is a method of monitoring the presence of pest or disrupting the mating of pest, said method comprising the steps of:
i) producing (Z9, E12)-tetradecadien-1-ol and/or an E12-fatty alcohol by the methods described herein;
ii) converting the E12-fatty alcohol and/or (Z9, E12)-tetradecadien-1-ol into an E12-fatty alcohol acetate and/or (Z9, E12)-tetradecadien-1-ol acetate, respectively;
iii) converting the E12-fatty alcohol and/or (Z9, E12)-tetradecadien-1-ol into an E12-fatty aldehyde and/or (Z9, E12)-tetradecadienal, respectively;
iv) formulating said E12-fatty alcohol, (Z9, E12)-tetradecadien-1-ol, E12-fatty alcohol acetate, (Z9, E12)-tetradecadien-1-ol acetate, E12-fatty aldehyde, and/or (Z9, E12)-tetradecadienal as a pheromone composition; and
v) employing said pheromone composition as an integrated pest management composition.

Also provided herein is a pheromone composition obtainable by a method comprising the following steps:
i) producing (Z9, E12)-tetradecadien-1-ol and/or an (E12)-fatty alcohol by the methods described herein;
ii) converting the (E12)-fatty alcohol and/or (Z9, E12)-tetradecadien-1-ol into an (E12)-fatty alcohol acetate and/or (Z9, E12)-tetradecadien-1-ol acetate, respectively;
iii) converting the (E12)-fatty alcohol and/or (Z9, E12)-tetradecadien-1-ol into an (E12)-fatty aldehyde and/or (Z9, E12)-tetradecadienal, respectively;
iv) formulating said (E12)-fatty alcohol, (Z9, E12)-tetradecadien-1-ol, E12-fatty alcohol acetate, (Z9, E12)-tetradecadien-1-ol acetate, (E12)-fatty aldehyde, and/or (Z9, E12)-tetradecadienal as a pheromone composition.

Also provided herein is a pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein the pheromone composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 80% biobased carbon, such as at least 85% biobased carbon, such as at least 90% biobased carbon, such as at least 95% biobased carbon, such 100% biobased carbon.

The relative amounts of the E12-fatty alcohol, the (Z9, E12)-tetradecadien-1-ol, the E12-fatty alcohol acetate, the (Z9, E12)-tetradecadien-1-ol acetate, the E12-fatty aldehyde, and/or the (Z9, E12)-tetradecadienal in the present pheromone compositions may vary depending on the nature of the crop and/or of the pest to be controlled; geographical variations may also exist. Determining the optimal relative amounts may thus require routine optimisation.

The pheromone composition may thus comprise between 1 and 100% E12-fatty alcohol, between 1 and 100% (Z9, E12)-tetradecadien-1-ol, between 1 and 100% E12-fatty alcohol acetate, between 1 and 100% (Z9, E12)-tetradecadien-1-ol acetate, between 1 and 100% E12-fatty aldehyde and/or between 1 and 100% (Z9, E12)-tetradecadienal.

In some embodiments, the ratio of (Z9, E12)-tetradecadien-1-ol to the E12-fatty alcohol, (Z9, E12)-tetradecadien-1-ol acetate to the E12-fatty alcohol acetate and/or (Z9, E12)-tetradecadienal to the E12-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to the E12-fatty alcohol in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol acetate to the E12-fatty alcohol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadienal to the E12-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to a Z9-fatty alcohol, (Z9, E12)-tetradecadien-1-ol acetate to a Z9-fatty alcohol acetate and/or (Z9, E12)-tetradecadienal to a Z9-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to a Z9-fatty alcohol in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol acetate to a Z9-fatty alcohol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadienal to a Z9-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to (Z9, E12)-tetradecadienal and/or (Z9, E12)-tetradecadien-1-ol to (Z9, E12)-tetradecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to (Z9, E12)-tetradecadienal in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the ratio of (Z9, E12)-tetradecadien-1-ol to (Z9, E12)-tetradecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the pheromone composition comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.

In some embodiments, the pheromone composition comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon.

In some embodiments, the pheromone composition comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.

In some embodiments, the pheromone composition comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.

Also provided herein is a pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein the pheromone composition has a radioactive ¹⁴C level of at least 20%.

In some embodiments, the pheromone composition has a radioactive ¹⁴C level of at least 30%, alternatively 40%, alternatively 50%, alternatively 60%, alternatively 70%, alternatively 75%, alternatively 80%, alternatively 85%, alternatively 90%, alternatively 95%.

In some embodiments, the pheromone composition has a radioactive ¹⁴C level of at least 80%, alternatively 85%, alternatively 90%, alternatively 95%, alternatively 96%, alternatively 97%, alternatively 98%, alternatively 99%.

In some embodiments, the pheromone composition has a radioactive ¹⁴C level between 30-100%, alternatively 40-100%, alternatively 50-100%, alternatively 60-100%, alternatively 70-100%, alternatively 75-100%, alternatively 80-100%, alternatively 85-100%, alternatively 90-100%, alternatively 95-100%.

In some embodiments, the pheromone composition has a radioactive ¹⁴C level between 80-100%, alternatively 85-100%, alternatively 90-100%, alternatively 95-100%, alternatively 96-100%, alternatively 97-100%, alternatively 98-100% carbon, alternatively 99-100%.

Also provided herein is a method for producing a pheromone composition according to the invention comprising the following steps:
i) producing (Z9, E12)-tetradecadien-1-ol and/or an E12-fatty alcohol by the method as disclosed herein;
ii) converting the E12-fatty alcohol and/or (Z9, E12)-tetradecadien-1-ol into an E12-fatty alcohol acetate and/or (Z9, E12)-tetradecadien-1-ol acetate, respectively;
iii) converting the E12-fatty alcohol and/or (Z9, E12)-tetradecadien-1-ol into an E12-fatty aldehyde and/or (Z9, E12)-tetradecadienal, respectively;
iv) formulating the E12-fatty alcohol, (Z9, E12)-tetradecadien-1-ol, E12-fatty alcohol acetate, (Z9, E12)-tetradecadien-1-ol acetate, E12-fatty aldehyde, and/or (Z9, E12)-tetradecadienal as a pheromone composition, optionally comprising one or more additional compounds such as a liquid or solid carrier or substrate;
v) optionally determining the biobased carbon content or radioactive ¹⁴C level of the pheromone composition.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. In one embodiment, said compositions are placed in a device, such as a pheromone dispenser, which diffuses the pheromone composition. The dispenser may for example release pheromones at a constant, pre-adjustable, rate. This can result in mating disruption, thereby preventing pest reproduction, or it can be used in combination with a trapping device to entrap the pests. Non-limiting examples of pests against which the present pheromone compositions can be used are: cotton bollworm (Helicoverpa armigera), striped stemborer (Chilo suppressalis), diamond back moth (Plutella xylostella), cabbage moth (Mamestra brassicae), large cabbage-heart caterpillar (Crocidolomia binotalis), European corn stalk borer (Sesamia nonagrioides), currant clearwing (Synanthedon tipuliformis), artichoke plume moth (Platyptilia carduidactylal), Indianmeal moth (Plodia interpunctella), beet armyworm (Spodoptera exigua), almond moth (Cadra cautella), Southern armyworm (Spodoptera eridania) and others. Accordingly, use of the present compositions on a culture can lead to increased crop yield, with substantially no environmental impact.

The relative amounts of fatty alcohols, fatty alcohol acetates and/or fatty aldehydes in the present pheromone compositions may vary depending on the nature of the crop and/or of the pest to be controlled; geographical variations may also exist. Determining the optimal relative amounts may thus require routine optimisation.

Examples of compositions used in pest control can be found in Kehat & Dunkelblum (1993) for H. armigera; in Alfaro et al. (2009) for C. suppressalis; in Eizaguirre et al. (2002) for S. nonagrioides; in Wu et al. (2012) for P. xylostella; in Bari et al. (2003) for P. carduidactyla; in Zhu et al. (1999) for P. interpunctella; in Wakamura (1987) for S. exigua; and in Brady et al. (1971) for C. cautella.

In some embodiments, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be in the form of a solution, a gel, a powder. The pheromone composition may be formulated so that it can be easily dispensed, as is known in the art.

The present mating disruption methods may be employed in fields of transgenic crops.

Also provided herein is a method for reducing or delaying the emergence of resistance to a pesticidal trait; the method may be an integrated resistance management method. Thus are disclosed preemptive and responsive methods to delay the development of resistance in a pest such as an insect, for example any of the insects listed herein, to a transgenic insecticidal crop and/or to chemical insecticide, i.e. a preemptive strategy. Also disclosed are methods to rescue one or more pest's susceptibility to transgenic insecticidal crops and/or chemical insecticides once resistance has developed, i.e. a responsive strategy. In some embodiments, the method comprises the application of a pheromone composition, such as obtained by the methods disclosed herein, to an agricultural area comprising a field population, wherein transgenic crops comprising one or more insecticidal traits such as transgenic insecticidal trais active against one of the insects listed herein, and optionally a refuge comprising crops devoid of insecticidal traits, to disrupt mating of the pest, thereby delaying the emergence of resistance to the insecticidal trait. The present compositions may thus be used in combination with any of the methods described in WO 2017/112887.

Also provided herein is a method for preventing or reducing crop damage from a pest such as an insect as listed herein. Such methods comprise applying mating disruption to a field by applying a pheromone composition as disclosed herein, and disrupting the expression of one or more target genes in one or more pests, thereby reducing or preventing crop damage in the field. Disruption of expression of one or more target genes can be achieved using RNAi, for example as described in WO 2017/205751. The present compositions may thus be used in combination with any of the methods described in WO 2017/205751.

### Pheromone Compounds

Also provided herein is a pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein the pheromone compound comprises at least 20% biobased carbon.

In some embodiments, the pheromone compound comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.

In some embodiments, the pheromone compound comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon.

In some embodiments, the pheromone compound comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.

In some embodiments, the pheromone compound comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.

Also provided herein is a pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein the pheromone compound has a radioactive ¹⁴C level of at least 20%.

In some embodiments, the pheromone compound has a radioactive ¹⁴C level of at least 30%, alternatively 40%, alternatively 50%, alternatively 60%, alternatively 70%, alternatively 75%, alternatively 80%, alternatively 85%, alternatively 90%, alternatively 95%.

In some embodiments, the pheromone compound has a radioactive ¹⁴C level of at least 80%, alternatively 85%, alternatively 90%, alternatively 95%, alternatively 96%, alternatively 97%, alternatively 98%, alternatively 99%.

In some embodiments, the pheromone compound has a radioactive ¹⁴C level between 30-100%, alternatively 40-100%, alternatively 50-100%, alternatively 60-100%, alternatively 70-100%, alternatively 75-100%, alternatively 80-100%, alternatively 85-100%, alternatively 90-100%, alternatively 95-100%.

In some embodiments, the pheromone compound has a radioactive ¹⁴C level between 80-100%, alternatively 85-100%, alternatively 90-100%, alternatively 95-100%, alternatively 96-100%, alternatively 97-100%, alternatively 98-100% carbon, alternatively 99-100%.

Further provided herein is a method for producing a pheromone compound according to the invention, in a yeast cell, said method comprising the steps of:
i) providing a yeast cell expressing a heterologous Δ12 fatty acyl-CoA desaturase,
ii) incubating said yeast cell in a medium under conditions allowing expression of a heterologous Δ12 fatty acyl-CoA desaturase,
   thereby producing the pheromone compound.

In some embodiments, the yeast cell expresses a further heterologous desaturase.

In some embodiments, the yeast cell further expresses at least one alcohol-forming fatty acyl-CoA reductase (FAR).

In some embodiments, the method further comprises a step of determining the biobased carbon content or radioactive ¹⁴C level of the pheromone compound.

### Biobased Products

“Biobased” products may be defined as products wherein:
1. the total carbon content of the product is at least 30%
2. the carbon content of a renewable raw material (biobased) is at least 20%.

Both fossil and renewable raw materials consist mainly of carbon (C). Carbon occurs in several isotopes. Isotope ¹⁴C is radioactive and occurs naturally in all living organisms (plants, animals, etc.) in a fixed relative concentration which is nearly identical to the relative ¹⁴C concentration in the atmosphere. At this concentration, the radioactivity level of ¹⁴C is 100%. Once an organism is no longer living, this concentration, and thus the radioactivity rate, decays with a half-life of approximately 5700 years. The radioactive ¹⁴C level of an unknown substance can therefore help determine how old the carbon contained in the substance is.

“Young” carbon (0 to 10 years) derived from renewable raw materials, such as plants or animals, has a relative isotope ¹⁴C concentration which is nearly identical to the relative ¹⁴C concentration in the atmosphere and the radioactive ¹⁴C level of such young carbon is thus about 100%.

“Old” carbon (millions of years) derived from synthetic or fossil (petrochemical) sources is greatly depleted from isotope ¹⁴C as the age of such synthetic and fossil sources far exceeds the half-life of isotope ¹⁴C which is approximately 5700 years. Hence, carbon derived from synthetic or fossil sources has a relative isotope ¹⁴C concentration around 0% and the radioactive ¹⁴C level of such old carbon is thus about 0%.

In one embodiment the term “radioactive ¹⁴C level” refer to the total radioactive ¹⁴C level of a given substance, product or composition, as defined above.

The isotope ¹⁴C method may be used to determine the concentration of young (renewable) materials in comparison with the concentration of old (fossil) resources. The carbon content of a renewable raw material is referred to as the “biobased carbon content”. The carbon content of a renewable raw material or the “biobased carbon content” may be determined as described below.

When measuring the biobased carbon content, the result may be reported as “% biobased carbon”. This indicates the percentage carbon from “natural” (plant or animal by-product) sources versus “synthetic” or “fossil” (petrochemical) sources. For reference, 100% biobased carbon indicates that a material is entirely sourced from plants or animal by-products and 0% biobased carbon indicates that a material did not contain any carbon from plants or animal by-products. A value in between represents a mixture of natural and fossil sources.

Example: If a product has a radioactive ¹⁴C level of 80%, it means that the product consists of 80% renewable and 20% fossil carbon (C). In other words, the product is 80% bio-based.

The analytical measurement may be cited as “percent modern carbon (pMC)”. This is the percentage of ¹⁴C measured in the sample relative to a modern reference standard (NIST 4990C). The % Biobased Carbon content is calculated from pMC by applying a small adjustment factor for ¹⁴C in carbon dioxide in air today. It is important to note is that all internationally recognized standards using ¹⁴C assume that the plant or biomass feedstocks were obtained from natural environments.

### EXAMPLES

### Example 1—Construction of BioBricks and Plasmids

All heterologous genes were synthesized by GeneArt (Life Technologies) in codon-optimized versions for Yarrowia lipolytica. All the genes were amplified by PCR using Phusion U Hot Start DNA Polymerase (ThermoFisher) to obtain the fragments for cloning into yeast expression vectors. The primers are listed in Table 1 and the resulting DNA fragments (BioBricks) are listed in Table 2. BioBricks labelled with “***Bsal” were obtained by a Bsal restriction digest of the indicated parent plasmid following the manufacturer instructions. The PCR products or restriction digest reaction were separated on a 1%-agarose gel containing Midori Green Advance (Nippon Genetics Europe GmbH). PCR/restriction digest products of the correct size were excised from the gel and purified using the Nucleospin Gel and PCR Clean-up kit (Macherey-Nagel).

Yeast vectors with USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into vectors by USER-cloning as described in (Holkenbrink, et al., 2018; Jensen, et al., EasyClone: method for iterative chromosomal integration of multiple genes in Saccharomyces cerevisiae, 2014). Plasmids marked with “ligation” (Table 3) were obtained by a DNA T4 ligation reaction including the indicated BioBricks following the manufacturer instructions. The USER/T4 ligase reaction was transformed into chemically competent E. coli DHα cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

Strains marked with “**” were constructed as follows. The indicated genes were amplified with gene-specific primers containing a 5′-overhang of “ACTTTTTGCAGTACUAACCGCAG” in the forward primer and a 3′-overhang of “CACGCGAU” in the reverse primer. The first “ATG” of the target gene sequence was omitted. These PCR products were cloned together with BB9454 either into integrative vectors or episomal vectors as described in (Holkenbrink, et al., 2018).

Episomal expression vectors for use in Saccharomyces cerevisiae were cloned and transformed as described in Jensen, 2014. The desaturases Desat59 and Pid12 were expressed under the control of the S. cerevisiae native promoters of genes TEF1 and TDH3, respectively.

TABLE 1
Primers

| | | NCBI accession | Hybridises |
|---|---|---|---|
| Primer ID | Template | number | at positions |
| PR10595 | Yali0C | NC_006069 | 1244252 . . . 1244265 |
| PR10604 | pCfB6630* | — | 3458 . . . 3498 |
| PR10607 | Yali0A | NC_006067 | 483924 . . . 483945 |
| PR10852 | Yali0E | NC_006071 | 784691 . . . 784713 |
| PR11110 | pCfB6681 | — | 2306 . . . 2336 |
| PR11111 | pCfB6681* | — | 5129 . . . 5152 |
| PR14148 | Yali0E | NC_006071 | 784691 . . . 784708 |
| PR15781 | Yali0A | NC_006067 | 2188556 . . . 2188574 |
| PR14279 | Yali0C | NC_006069 | 1244254 . . . 1244265 |
| PR14620 | Yali0A | NC_006067 | 2188565 . . . 2188546 |
| PR15788 | Yali0A | NC_006067 | 456893 . . . 456922 |
| PR15789 | Yali0F | NC_006072 | 1948664 . . . 1948678 |
| PR15930 | Yali0C | NC_006069 | 1243743 . . . 1243762 |
| PR16594 | SEQ ID NO: 19 | — | 6 . . . 21 |
| PR16595 | SEQ ID NO: 19 | — | 1348 . . . 1368 |
| PR18214 | Yali0C | NC_006069 | 1243743 . . . 1243761 |
| PR23166 | Yali0E | NC_006071 | 2086416 . . . 2086402 |
| PR23167 | Yali0E | NC_006071 | 2085907 . . . 2085919 |
| PR23168 | Yali0E | NC_006071 | 2086480 . . . 2086497 |
| PR23169 | Yali0E | NC_006071 | 2087001 . . . 2086986 |
| PR23190 | Yali0E | NC_006071 | 2086449 . . . 2086430 |
| PR23191 | Yali0E | NC_006071 | 2086430 . . . 2086449 |
| PR23880 | SEQ ID NO: 21 | — | 4 . . . 20 |
| PR23882 | SEQ ID NO: 21 | — | 1345 . . . 1365 |
| PR23978 | SEQ ID NO: 25 | — | 4 . . . 17 |
| PR23979 | SEQ ID NO: 25 | — | 1342 . . . 1365 |
| PR24187 | SEQ ID NO: 3 | — | 4 . . . 18 |
| PR24188 | SEQ ID NO: 3 | — | 1131 . . . 1146 |
| PR25093 | | | SED ID NO: 33 |
| PR25094 | | | SEQ ID NO: 34 |
| PR25222 | Yali0F | NC_006072 | 823328 . . . 823348 |
| PR25223 | Yali0F | NC_006072 | 823833 . . . 823815 |
| PR25224 | Yali0F | NC_006072 | 823920 . . . 823938 |
| PR25225 | Yali0F | NC_006072 | 824426 . . . 824408 |
| PR25295 | Yali0F | NC_006072 | 823861 . . . 823842 |
| PR25296 | Yali0F | NC_006072 | 823842 . . . 823861 |
| PR25309 | Yali0C | NC_006069 | 1244237 . . . 1244223 |
| PR25314 | | | SEQ ID NO: 35 |
| PR25381 | | | SEQ ID NO: 36 |
| PR25419 | SEQ ID NO: 1 | — | 4 . . . 20 |
| PR25420 | SEQ ID NO: 1 | — | 993 . . . 1011 |
| PR25512 | SEQ ID NO: 27 | — | 4 . . . 20 |
| PR25513 | SEQ ID NO: 27 | — | 1589 . . . 1608 |
| PR25514 | SEQ ID NO: 42 | — | 4 . . . 20 |
| PR25515 | SEQ ID NO: 42 | — | 1556 . . . 1572 |
| PR25518 | SEQ ID NO: 44 | — | 4 . . . 20 |
| PR25519 | SEQ ID NO: 44 | — | 1518 . . . 1536 |
| PR25526 | SEQ ID NO: 23 | — | 4 . . . 20 |
| PR25527 | SEQ ID NO: 23 | — | 1362 . . . 1380 |
| PR25456 | SEQ ID NO: 46 | — | 4 . . . 19 |
| PR25458 | SEQ ID NO: 46 | — | 1015 . . . 1035 |
| PR25460 | SEQ ID NO: 48 | — | 4 . . . 21 |
| PR25462 | SEQ ID NO: 48 | — | 1943 . . . 1959 |
| PR25457 | SEQ ID NO: 50 | — | 4 . . . 20 |
| PR25459 | SEQ ID NO: 50 | — | 849 . . . 967 |
| PR25461 | SEQ ID NO: 48 | — | 4 . . . 19 |
| PR25463 | SEQ ID NO: 46 | — | 1130 . . . 1146 |
| PR26756 | SEQ ID NO: 54 | — | 4 . . . 23 |
| PR26757 | SEQ ID NO: 54 | — | 998 . . . 1017 |

TABLE 2
DNA fragments (BioBricks) obtained by PCR
using the indicated template and primers

| BioBrick | | | Forward | Reverse |
|---|---|---|---|---|
| ID | Description | Template | primer | primer |
| BB2313* | Up- and downstream genomic | | | |
| | region of Fas2pl1220 | | | |
| BB9611 | Spodoptera exigua fatty acyl | SEQ ID NO: 23 | PR25526 | PR25527 |
| | reductase | | | |
| BB1135 | Vector backbone | pCfB6681* | PR11110 | PR11111 |
| BB1631 | Regions downstream of Y. | pCfB4586* | PR14148 | PR15781 |
| | lipolytica pex20 and lip2 | | | |
| BB1635 | Promoter for gRNA expression | pCfB4589* | PR10607 | PR15788 |
| BB1636 | Terminator region for gRNA | pCfB4589* | PR15789 | PR10604 |
| | expression | | | |
| BB1688 | region upstream of Y. | ST4840** | PR14279 | PR15930 |
| | lipolytica tef1 | | | |
| BB1740 | Helicoverpa armigera fatty | SEQ ID NO: 19 | PR16594 | PR16595 |
| | acyl reductase | | | |
| BB2093 | Y. lipolytica tef1 promoter | ST4840** | PR10595 | PR18214 |
| BB8679 | Genomic region upstream of | ST4840** | PR23167 | PR23166 |
| | integration site | | | |
| BB8680 | Genomic region downstream | ST4840** | PR23168 | PR23169 |
| | of integration site | | | |
| BB8907 | Spodoptera exigua fatty acyl | SEQ ID NO: 21 | PR23880 | PR23882 |
| | reductase | | | |
| BB8929 | Spodoptera litura fatty acyl | SEQ ID NO: 25 | PR23978 | PR23979 |
| | reductase | | | |
| BB8955 | Regions downstream of Y. | pBP8662 | PR10852 | PR14620 |
| | lipolytica pex20 and lip2 | | | |
| BB8971 | Drosophila virilis desaturase | SEQ ID NO: 3 | PR24187 | PR24188 |
| BB9431 | Genomic region upstream of | ST4840** | PR25222 | PR25223 |
| | integration site | | | |
| BB9432 | Genomic region downstream | ST4840** | PR25224 | PR25225 |
| | of integration site | | | |
| BB9450 | Regions downstream of Y. | pBP9002 | PR25093 | PR25314 |
| | lipolytica pex20 and lip2 | | | |
| BB9454 | region upstream of Y. | ST4840** | PR25309 | PR18214 |
| | lipolytica tef1 | | | |
| BB9506 | Regions downstream of Y. | pBP9002 | PR25381 | PR25094 |
| | lipolytica pex20 and lip2 | | | |
| BB9527 | Plodia interpunctella desaturase | SEQ ID NO: 1 | PR25419 | PR25420 |
| BB9588*** | Vector backbone | pBP9683*** | | |
| BB9603*** | region downstream of pex20, | pBP9723*** | | |
| | region upstream of tef1 and | | | |
| | Plodia interpunctella desaturase | | | |
| BB9604 | Plodia interpunctella fatty acyl | SEQ ID NO: 27 | PR25512 | PR25513 |
| | reductase | | | |
| BB9605 | Plodia interpunctella fatty acyl | SEQ ID NO: 42 | PR25514 | PR25515 |
| | reductase | | | |
| BB9607 | Plodia interpunctella fatty acyl | SEQ ID NO: 44 | PR25518 | PR25519 |
| | reductase | | | |
| BB9562 | Plodia interpunctella desaturase | SEQ ID NO: 46 | PR25456 | PR25457 |
| BB9563 | Plodia interpunctella desaturase | SEQ ID NO: 48 | PR25458 | PR25459 |
| BB9564 | Plodia interpunctella desaturase | SEQ ID NO: 50 | PR25460 | PR25461 |
| BB9565 | Plodia interpunctella desaturase | SEQ ID NO: 52 | PR25462 | PR25463 |
| BB10097 | Cadra cautella desaturase | SEQ ID NO: 54 | PR26756 | PR26757 |

*Holkenbrink et al., 2018
**Holkenbrink et al., 2020
***Bsal

TABLE 3
Vectors

| | | Parent | |
|---|---|---|---|
| Plasmid ID | Resistance | plasmid | BioBricks |
| pCfB7088* | | | |
| pBP8575 | Nourseothricin | pCfB3405* | BB1635, BB1636, |
| | | | PR23190, PR23191 |
| pBP8662 | — | | BB1135, BB8679, |
| | | | BB8680, BB1631 |
| pBP9002 | Nourseothricin | pCfB3405* | BB8955 |
| pBP9374 | — | pBP8662 | BB2093, BB8971 |
| pBP9560 | Nourseothricin | pBP9002 | BB1688, BB1740 |
| pBP9683 | — | | BB1135, BB9450, |
| | | | BB9431, BB9432 |
| pBP9684 | Hygromycin | pCfB3431* | BB1635, BB1636, |
| | | | PR25295, PR25296 |
| pBP9698 | Nourseothricin | pCfB3405* | BB9506 |
| pBP9699 | Hygromycin | pCfB3431** | BB9506 |
| pBP9723 | Hygromycin | pBP9699 | BB9454, BB9527 |
| pBP9819*** | — | | BB9588, BB9603 |
| pBP9820 | Nourseothricin | pBP9698 | BB9454, BB8907 |
| pBP9821 | Nourseothricin | pBP9698 | BB9454, BB9611 |
| pBP9822 | Nourseothricin | pBP9698 | BB9454, BB8929 |
| pBP9823 | Nourseothricin | pBP9698 | BB9454, BB9604 |
| pBP9824 | Nourseothricin | pBP9698 | BB9454, BB9605 |
| pBP9826 | Nourseothricin | pBP9698 | BB9454, BB9607 |
| pCfB6630* | | | |
| pCfB7980 | — | pCfB6371** | BB1688, BB1740 |
| pBP8676 | Hygromycin | pCfB3431** | BB1635, BB1636, |
| | | | PR23190, PR23191 |
| pBP9002 | Nourseothricin | pCfB3405* | BB8955 |
| pBP9374 | — | pBP8662 | BB2093, BB8971 |
| pBP9752 | Nourseothricin | pBP9698 | BB9454, BB9562 |
| pBP9753 | Nourseothricin | pBP9698 | BB9454, BB9563 |
| pBP9754 | Nourseothricin | pBP9698 | BB9454, BB9564 |
| pBP9755 | Nourseothricin | pBP9698 | BB9454, BB9565 |
| pBP8662 | — | | BB1135, BB8679, |
| | | | BB8680, BB1631 |
| pBP10481 | — | pBP9683 | BB9454, BB10097 |

*Holkenbrink et al., 2018
**Holkenbrink et al., 2020
***Ligation

### Example 2—Construction of Yeast Strains

Yeast strains were constructed by transformation of DNA vectors as described in Holkenbrink et al., 2018; Jensen et al., 2014. Integrative vectors were linearized with FastDigest NotI prior to transformation. When needed, helper vectors to promote the integration into specific genomic regions were co-transformed with the integrative plasmid or DNA repair fragments listed in Table 3. Strains were selected on yeast peptone dextrose (YPD) agar with appropriate antibiotics selection or on synthetic drop-out medium (Sigma-Adrich) lacking a specific amino acid. Correct genotype was confirmed by colony PCR and when needed by sequencing. The resulting strains are listed in Table 4.

TABLE 4
Yeast strains

| Name | Overexpressed gene | Parent Strain | DNA |
|---|---|---|---|
| ST7982 | — | ST6629** | pCfB7088 BB2313 |
| ST10628 | Desat61 | ST7982 | pBP9374 pBP8575 |
| ST10870 | Desat61 Pid12 | ST10628 | pBP9819 pBP9684 |
| ST10871 | — | ST10870 | pBP9002 |
| ST10872 | Desat61 Pid12 FAR1 | ST10870 | pBP9560 |
| ST10873 | Desat61 Pid12 FAR16 | ST10870 | pBP9820 |
| ST10874 | Desat61 Pid12 FAR17 | ST10870 | pBP9821 |
| ST10875 | Desat61 Pid12 FAR19 | ST10870 | pBP9822 |
| ST10876 | Desat61 Pid12 FAR28 | ST10870 | pBP9823 |
| ST10877 | Desat61 Pid12 FAR29 | ST10870 | pBP9824 |
| ST10879 | Desat61 Pid12 FAR31 | ST10870 | pBP9826 |
| ST8520 | FAR1 | ST7892 | pBP7980 pBP6630 |
| ST10661 | FAR1 Desat61 | ST8520 | pBP9374 pBP8676 |
| ST10629 | Desat61 Pid12 FAR1 | ST10629 | pBP9002 |
| ST11190 | FAR1 Desat61 Desat65 | ST10629 | pBP9752 |
| ST11191 | FAR1 Desat61 Desat66 | ST10629 | pBP9753 |
| ST11192 | FAR1 Desat61 Desat67 | ST10629 | pBP9754 |
| ST11193 | FAR1 Desat61 Desat68 | ST10629 | pBP9755 |
| ST12390 | Desat61 EcauDes12 | ST10628 | pBP10481 |
| | | | pBP9684 |
| ST10872** | FAR1 | ST10870 | |
| ST12294** | FAR44 | ST10870 | |
| ST12295** | FAR48 | ST10870 | |
| ST12296** | FAR49 | ST10870 | |
| ST12299** | FAR38 | ST10870 | |
| ST12271** | FAR4 | ST10870 | |
| ST12273** | FAR6 | ST10870 | |
| ST12274** | FAR8 | ST10870 | |
| ST12277** | FAR12 | ST10870 | |
| ST12279** | FAR11 | ST10870 | |
| ST12272** | FAR5 | ST10870 | |
| ST12614** | Pid12 | ST7982 | |
| ST12630** | Pid12 | ST12614 | |
| ST12631** | Pid12 Desat24 | ST12614 | |
| ST12632** | Pid12 Desat56 | ST12614 | |
| ST12634** | Pid12 Desat60 | ST12614 | |
| ST12635** | Pid12 Desat61 | ST12614 | |
| ST12640** | Pid12 Desat74 | ST12614 | |
| ST12039** | EcauDes12 | ST7982 | |
| ST12256** | EcauDes12 Desat24 | ST12039 | |
| ST12257** | EcauDes12 Desat56 | ST12039 | |
| ST12260** | EcauDes12 Desat60 | ST12039 | |
| ST12261** | EcauDes12 Desat61 | ST12039 | |
| ST12266** | EcauDes12 Desat74 | ST12039 | |
| ST12270** | EcauDes12 | ST12039 | |
| ST10628 | Desat61 | | |
| ST12366 | Desat61 Pid12 | ST10628 | |
| ST12368 | Desat61 Desat80 | ST10628 | |
| ST12367 | Desat61 Ecaudes12 | ST10628 | |
| ST12374 | Desat61 Desat81 | ST10628 | |
| ST12379 | Desat61 Desat82 | ST10628 | |
| CEN.PK102-5B*** | | | |
| ST12587 | Pid12 | CEN.PK102-5B | |
| ST12585 | Desat59 Pid12 | CEN.PK102-5B | |
| ST12596** | Desat61 Eku_d12 | ST7982 | |
| ST10748** | Desat61 | ST7982 | |

*Holkenbrink et al., 2020
**For strain description, see Example 1
***Euroscarf (Germany)

### Example 3—Cultivation of Strains and Analysis of Fatty Alcohols and Fatty Acid Methyl Esters (FAME)

Yarrowia lipolytica strains were inoculated from a YPD agar plate (10 g/L yeast extract, 10 g/L peptone, 20 g/L glucose, 15 g/L agar agar) to an initial OD600 of 0.1-0.2 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 22 h, the plates were centrifuged for 5 min at 4° C. and 3,000×g. The supernatant was discarded and the cells were resuspended in 1.25 mL production medium per well (50 g/L glycerol, 5 g/L yeast extract, 4 g/L KH₂PO₄, 1.5 g/L MgSO₄, 0.2 g/L NaCl, 0.265 g/L CaCl₂·2H₂O, 2 mL/L trace elements solution: 4.5 g/L CaCl₂·2H₂O, 4.5 g/L ZnSO₄·7H2O, 3 g/L FeSO₄·7H₂O, 1 g/L H₃BO₃, 1 g/L MnCl₂·4H₂O, 0.4 g/L N Na₂MoO₄·2H₂O, 0.3 g/L CoCl₂·6H₂O, 0.1 g/L CuSO₄·5H₂O, 0.1 g/L KI, 15 g/L EDTA). The media was supplemented with antibiotics if necessary. The plate was incubated for 28 hours at 28° C., shaken at 300 rpm.

Saccharomyces cerevisiae strains were inoculated from synthetic drop-out agar plates (lacking uracil, leucine and histidine) to an initial OD600 of 0.1-0.2 into 2.5 mL synthetic drop-out medium (lacking uracil, leucine and histidine) supplemented with 2% glucose, 1% tergitol, oleic acid (5.6 ml/L) and methyl myristate (2 ml/L) in 24 well-plates (EnzyScreen).

For analysis of fatty alcohols, 1 ml of cultivation broth was centrifuged and the supernatant was aspirated and discarded. 1 ml of ethyl acetate:ethanol (84:15) and 10 μL of Z10-17:Me (2 mg/mL) as internal standard was added to the cell pellet. The samples were vortexed for 20 sec and incubated for 1 h at room temperature, followed by 5 min of vortexing. 300 μL of H2O was added to each sample. The samples were vortexed and centrifuged for 5 min at 21° C. and 3,000×g. The upper organic phase was analyzed via gas chromatography-mass spectrometry (GC-MS). GC-MS analyses were performed on an Agilent 7820A GC coupled to a mass selective detector Agilent 5977B. The GC was equipped with an DB Fatwax column (30 m×0.25 mm×0.25 μm), and helium was used as carrier gas. The MS was operated in electron impact mode (70 eV), scanning between m/z 30 and 400, and the injector was configured in split mode 20:1 at 220° C. Oven temperature was set to 80° C. for 1 min, then increased at a rate of 20° C./min to 210° C., followed by a hold at 210° C. for 7 min, and then increased at a rate of 20 C/min to 230° C. Compounds were identified by comparison of retention times and mass spectra of the reference compounds. Data were analyzed by the Agilent Masshunter software. The concentrations of fatty alcohols were calculated based on standard calibration curves prepared with reference standards.

For analysis of the fatty acids, 1 mL of each vial was harvested by centrifugation for 5 min at 4° C. and 3,000×g. Each pellet was extracted with 1000 μL 1M HCl in Methanol (anhydrous). The samples were vortexed for 20 sec and placed in the 80° C. water bath for 2 h. The samples were vortexed every 30 min for 10 sec. After cooling down of the samples to room temperature, 1000 μL of 1M NaOH in Methanol (anhydrous), 500 μL of NaCl saturated H2O, 990 μL of hexane and 10 μL of Z10-17:Me (2 mg/mL) as internal standard were added. The samples were vortexed and centrifuged for 5 min at 21° C. and 3,000×g. The upper organic phase was analyzed via GC-MS as described above.

### Example 4—Production of Z9, E12-14:CoA in the Yeast Yarrowia lipolytica

The ΔZ9-desaturase Desat61 from Drosophila virilis was expressed either alone (ST10628) or in combination with Pid12 desaturase from Plodia interpunctella (ST10870) in Yarrowia lipolytica strain ST7982 (Petkevicius et al., 2021) to give correspondingly strains ST10628 and ST10870. The strains were cultivated and fatty acids were extracted as described in Example 3.

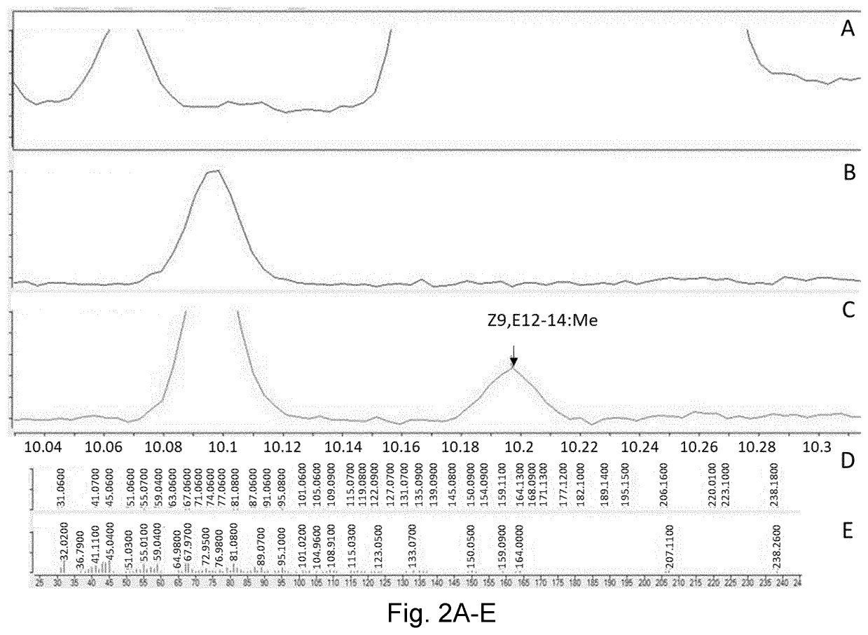

Strain ST10628, expressing the ΔZ9-desaturase Desat61, gave Z9-14:Me (FIG. 2B and Table 4). Strain ST10870, expressing both ΔZ9-desaturase Desat61 and the newly identified Pid12 desaturase, gave both, Z9-14:Me and a double unsaturated C14-fatty acid methyl ester. The double-unsaturated C14 fatty acid methyl ester eluted at the same retention time (10.2 min) as the analytical standard of Z9, E12:Me (FIG. 2A,C) and displayed a similar mass spectrum as the standard (FIG. 2D,E). The titers of Z9-14:Me and Z9, E12-14:Me are listed in Table 5.

TABLE 5
Production of Z9, E12-14:Me in the yeast Yarrowia lipolytica

| | Desaturases | Z9-14:Me | Z9, E12-14:Me |
|---|---|---|---|
| Strain ID | expressed | (mg/L) | (mg/L) |
| ST10628 | Desat61 | 27 | 0 |
| ST10870 | Desat61 Pid12 | 40 | 4 |

### Example 5—Production of Z9, E12-14:OH in Yarrowia lipolytica

The desaturase Desat61 from Drosophila virilis and Pid12 desaturase from Plodia interpunctella were expressed in Y. lipolytica strain ST7982 (Petkevicius et al., 2021) either alone or in combination with fatty acyl reductases from different insect species. The strains were cultivated and fatty alcohols were extracted as described in Example 3.

Strains ST10872 to ST10879, expressing the fatty acyl reductases FAR1 from Helicoverpa armigera, FAR16 from Spodoptera exigua, FAR17 from Spodoptera exigua, FAR19 from Spodoptera litura and FAR28, FAR29 and PiFAR31 from Plodia interpunctella, respectively, produced both Z9-14:OH and the target compound Z9, E12-14:OH (Table 6). The strain ST10875, expressing FAR19 produced the highest titer with 0.88 mg/L of Z9, E12-14:OH. Strains ST10877 and ST10879, expressing FAR29 and FAR31 produced only minor amounts of Z9-14:OH but no Z9, E12-14:OH.

TABLE 6
Production of Z9, E12-14:OH in Yarrowia lipolytica

| | | Z9-14:OH | Z9, E12-14:OH |
|---|---|---|---|
| Strain ID | Genes expressed | (mg/L) | (mg/L) |
| ST10871 | Desat61 Pid12 | 0 ± 0 | 0 ± 0 |
| ST10872 | Desat61 Pid12 FAR1 | 91 ± 13 | 0.65 ± 0.1 |
| ST10873 | Desat61 Pid12 FAR16 | 119 ± 9 | 0.76 ± 0.04 |
| ST10874 | Desat61 Pid12 FAR17 | 6 ± 2 | 0.04 ± 0.01 |
| ST10875 | Desat61 Pid12 FAR19 | 127 ± 8 | 0.88 ± 0.04 |
| ST10876 | Desat61 Pid12 FAR28 | 3.3 ± 1.5 | 0.03 ± 0.01 |
| ST10877 | Desat61 Pid12 FAR29 | 1.7 ± 0.4 | 0 ± 0 |
| ST10879 | Desat61 Pid12 FAR31 | 0.4 ± 0.5 | 0 ± 0 |

### Example 6—Expression of P. Interpunctella Desaturases in Yarrowia lipolytica

Strains ST10629 and ST11190 to ST11193 express the ΔZ9 desaturase Desat6l, the fatty acyl reductase FAR1 and various desaturase genes from Plodia interpunctella. Strain ST10661 serves as a control strain only expressing Desat6l and FAR1.

All strains tested produced Z9-14:OH but only the strain expressing Pid12, ST10629, produced Z9, E12-14:OH at a titer of 0.6 mg/l.

TABLE 6
Production of Z9, E12-14:OH in Yarrowia lipolytica

| | | Z9-14:OH | Z9, E12-14:OH |
|---|---|---|---|
| Strain ID | Genes expressed | (mg/L) | (mg/L) |
| ST10661 | FAR1 Desat61 | 182 ± 26 | 0 ± 0 |
| ST10629 | Desat61 Pid12 FAR1 | 102 ± 6 | 0.6 ± 0.1 |
| ST11190 | FAR1 Desat61 Desat65 | 70 ± 60 | 0 ± 0 |
| ST11191 | FAR1 Desat61 Desat66 | 134 ± 5 | 0 ± 0 |
| ST11192 | FAR1 Desat61 Desat67 | 193 ± 43 | 0 ± 0 |
| ST11193 | FAR1 Desat61 Desat68 | 154 ± 43 | 0 ± 0 |

### Example 7—Production of Z9, E12-14:CoA and Z9, E12-14:OH in the Yeast Saccharomyces cerevisiae

The ΔZ9 desaturase Desat59 or Desat6l or another ΔZ9 desaturase, the Pid12 desaturase and a fatty acyl reductase can be cloned into Saccharomyces cerevisiae gene expression vectors and transformed into S. cerevisiae as described in Jensen et al., 2014. Strain cultivation and sample extraction can be performed as in Example 3. Strains expressing a ΔZ9 desaturase and Pid12 desaturase from Plodia interpunctella produce Z9, E12-14:Me, while strains additionally expressing a fatty acyl reductase gene produce Z9, E12-14:OH.

### Example 8—Production of Z9, E12-14:OH in Yarrowia lipolytica Using Other Fatty Acyl-CoA Reductases

The desaturase Desat61 from Drosophila virilis and Pid12 desaturase from Plodia interpunctella were expressed in Y. lipolytica strain ST7982 (Petkevicius et al., 2021), either alone or in combination with fatty acyl reductase FAR32 from Plodia interpunctella. The strains were cultivated and fatty alcohols were extracted as described in Example 3.

### Example 9—Measurement of Biobased Carbon Content

The biobased carbon content of pheromone compounds according to the invention is measured by using the analytical measurement that may be cited as “percent modern carbon (pMC)”. This is the percentage of isotope ¹⁴C measured in the sample relative to a modern reference standard (NIST 4990C). The % biobased carbon content is calculated from pMC by applying a small adjustment factor for isotope ¹⁴C in carbon dioxide in air today.

The pheromone compounds according to the invention have a biobased carbon content above 80%.

### Example 10—Production of Z9, E12-14:CoA in Yarrowia lipolytica

The ΔZ9-desaturase Desat61 from Drosophila virilis is expressed either alone (ST10628) or in combination with EcauDes12 desaturase from Cadra cautella (ST12930) in Yarrowia lipolytica strain ST7982 (Petkevicius et al., 2021) to give correspondingly strains ST10628 and ST12930. The strains are cultivated and fatty acids are extracted as described in Example 3.

The derivatised extract of strain ST10628 contained Z9-14:Me, while the derivatised extract of strain ST12930 contained both Z9-14:Me and Z9,E12-14:Me.

### Example 11—Production of Z9, E12-14:CoA in the Yeast Saccharomyces cerevisiae

The ΔZ9 desaturase Desat59 was either expressed alone or in combination with Pid12 in S. cerevisiae as described in Jensen et al., 2014. Strain cultivation and sample extraction were performed as in Example 3. The derivatised extract of strain ST12585 co-expressing Desat59 and Pid12 contained 0.05±0.00 mg/L of Z9,E12-14:Me while the extract of strain ST12587 solely expressing Desat59 did not contain any Z9,E12-14:Me.

### Example 12—Preparation of Z9, E12-14:OAc by Acetylation of Z9, E12-14:OH

The acetylation reaction was carried out in a glass round bottom flask equipped with a magnetic stirrer. The reaction flask was loaded with 2 g of Z9, E12-14:OH (91% purity; 9.5 mmol) and 1.06 g acetic anhydride (99% purity, 10.5 mmol, 1.1 equivalents). The reaction mixture was heated to 80° C. under constant mixing. After 2 hours GC analysis showed full alcohol consumption.

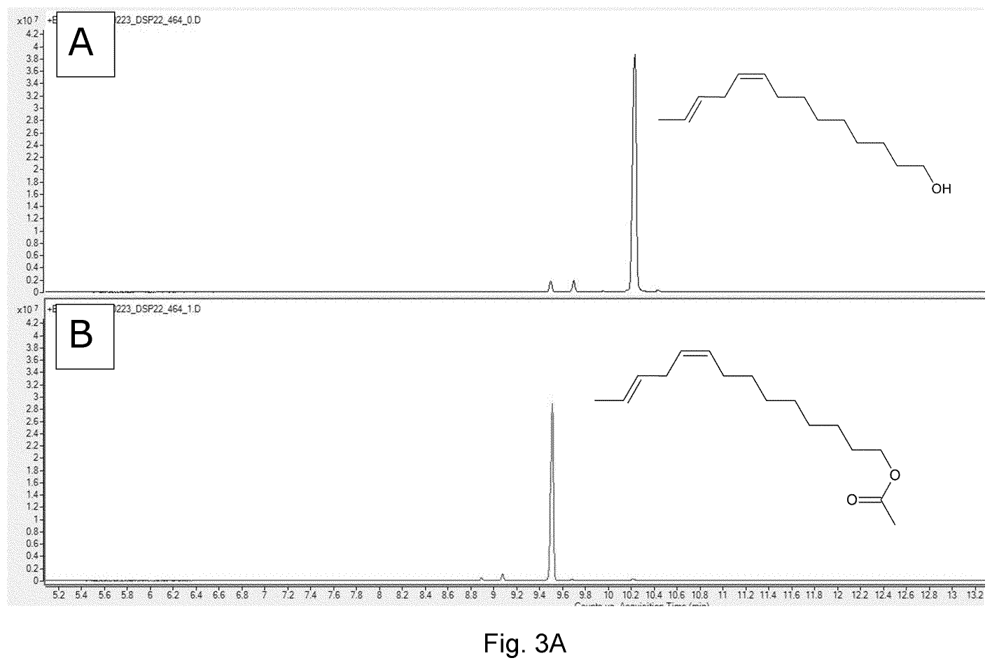

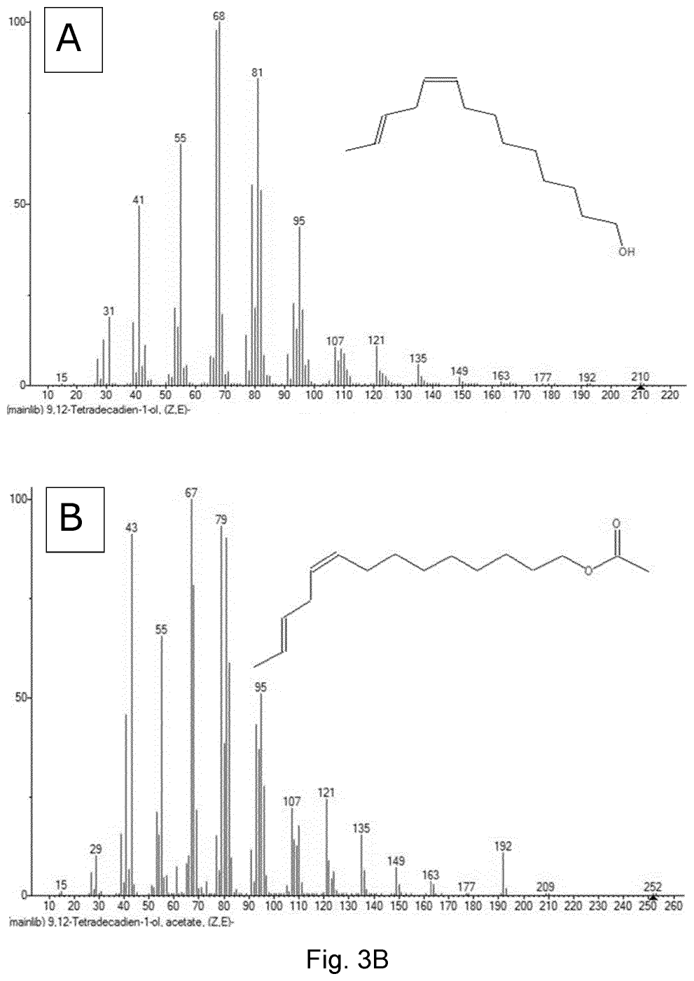

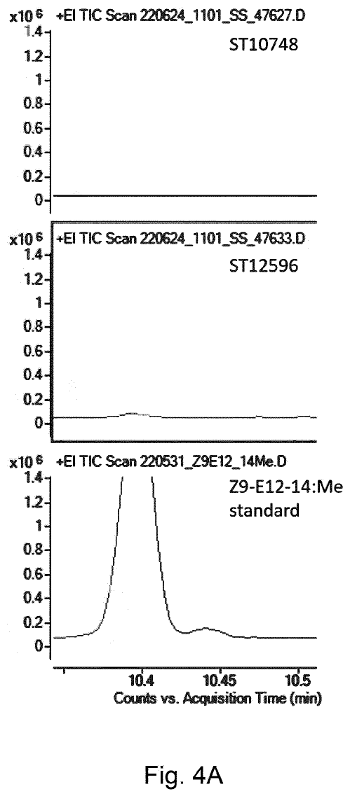

The acetic acid byproduct as well as the unreacted acetic anhydride were evaporated at reduced pressure yielding 2.28 g of a colorless oil. GC-MS analysis confirmed the structure of the product as Z9, E12-14:OAc pure at 92% (95% yield) (FIGS. 3 A and B).

### Example 13—Production of Z9,E12-14:OH in Yarrowia lipolytica Using Alternative FAR

The ΔZ9 desaturase Desat61 and Δ12 desaturase Pid12 were co-expressed with various fatty acyl-CoA reductases originating from various organisms individually. The strains were cultivated and fatty alcohols were extracted as described in Example 3, with the exception that the cells were incubated for 47 hours in YPG medium instead of 22 hours.

Extracts of strains ST10872, ST12294-ST12296, ST12299, ST12271-ST12274, ST12777 and ST12779 contained >0.01 mg/L of Z9,E12-14:OH (as indicated with “+” in the table below).

| Strain ID | FAR | Z9, E12-14:OH (mg/L) |
|---|---|---|
| ST10872 | FAR1 | + |
| ST12294 | FAR44 | + |
| ST12295 | FAR48 | + |
| ST12296 | FAR49 | + |
| ST12299 | FAR38 | + |
| ST12271 | FAR4 | + |
| ST12273 | FAR6 | + |
| ST12274 | FAR8 | + |
| ST12277 | FAR12 | + |
| ST12279 | FAR11 | + |
| ST12272 | FAR5 | + |

### Example 14—Production of Z9,E12-14:Acid in Yarrowia lipolytica Using Alternative Δ9 Desaturases

The Δ12 desaturases Pid12 and EcauDes12 were individually co-expressed with various Δ9 desaturases originating from various organisms. The strains were cultivated and FAME were extracted as described in Example 3, with the exception that the cells were incubated for 47 hours in YPG medium instead of 22 hours.

The lowest titer of Z9,E12-14:Me (>0 mg/L indicated with “+” in the table below) was detected in the extract of strain ST12630, which solely expressed Pid12. In this strain the Z9-14:CoA, the substrate for the Δ12 desaturation reaction, is produced as a by-product by the yeast native desaturase OLE1.

Strains ST12631, ST12632, ST12634, ST12635, ST12640, ST12256, ST12257, ST12260, ST12261 and ST12266, expressed additionally to the Δ12 desaturases heterologous Δ9 desaturases. Derivatized extracts of these strains contained higher levels of Z9,E12-14:Me (≥0.02 mg/L as indicated with “++” in the table below) than extracts of strain ST12630.

| | delta12 | delta9 | | |
|---|---|---|---|---|
| Strain ID | desaturase | desaturase | Z9-14:Me | Z9, E12-14:Me |
| ST12630 | Pid12 | Native Ole1 | + | + |
| ST12631 | Pid12 | Desat24 | ++ | ++ |
| ST12632 | Pid12 | Desat56 | ++ | ++ |
| ST12634 | Pid12 | Desat60 | ++ | ++ |
| ST12635 | Pid12 | Desat61 | ++ | ++ |
| ST12640 | Pid12 | Desat74 | ++ | ++ |
| ST12256 | EcauDes12 | Desat24 | ++ | ++ |
| ST12257 | EcauDes12 | Desat56 | ++ | ++ |
| ST12260 | EcauDes12 | Desat60 | ++ | ++ |
| ST12261 | EcauDes12 | Desat61 | ++ | ++ |
| ST12266 | EcauDes12 | Desat74 | ++ | ++ |

### Example 15—Production of Z9, E12-14:Acid in Yarrowia lipolytica Using Alternative Δ12 Desaturases

Fusion-protein variants of Pid12 and EcauDes12 were designed and tested. In these variants the N- and C-termini of the Δ12 desaturases Pid12 and EcauDes12 were replaced with the N- and C-termini of either the Plodia interpunctella desaturase Desat65 or the Amyelois transitella desaturase Desat16. The sequence identities of the fusion proteins can be seen in the table below. The strains were cultivated and FAME were extracted as described in Example 3, with the exception that the cells were incubated for 47 hours in YPG medium instead of 22 hours.

Strain ST12368 expressing a variant of Pid12 in which the N- and C-terminus (N-terminus amino acid 1-30, C-terminus amino acid 300-335) is replaced with those of Desat65 (N-terminus amino acid 1-29, C-terminus amino acid 299-344) produced similar amounts of Z9,E12-14:Me (>0.02 mg/L as indicated with “+” in the table below) to strains ST12366 and ST12367 expressing the unmodified Pid12 and EcauDes12, respectively. The same was observed for strains ST12374 and ST12379 which express variants of EcauDes12 in which the N- and C-terminus (N-terminus amino acid 1-30, C-terminus amino acid 300-338) are replaced with those of desaturase Desat65 (N-terminus amino acid 1-29, C-terminus amino acid 299-344) and Desat16 (N-terminus amino acid 1-20; C-terminus amino acid: 302-326), respectively.

Sequence identity percentage between the proteins shown in the table.

| | | 1 | 2 | 3 | 4 | 5 |
|---|---|---|---|---|---|---|
| EcauDes12 | 1 | | 90.46 | 89.97 | 79.65 | 80.92 |
| EcauDes12_N-term + | 2 | 90.46 | | 90.99 | 78.61 | 90.14 |
| Desat65_C-term | | | | | | |
| EcauDes12_N-term + | 3 | 89.97 | 90.99 | | 80.12 | 81.16 |
| Desat16_C-term | | | | | | |
| Pid12 | 4 | 79.65 | 78.61 | 80.12 | | 87.86 |
| Pid12_N-term + | 5 | 80.92 | 90.14 | 81.16 | 87.86 | |
| Desat65_C-term | | | | | | |

| Strain ID | delta12 desaturase | Avg Z9, E12-14:Me |
|---|---|---|
| ST12366 | Pid12 | + |
| ST12368 | Pid12_N-term + Desat65_C-term | + |
| ST12367 | EcauDes12 | + |
| ST12374 | EcauDes12_N-term + Desat65_C-term | + |
| ST12379 | EcauDes12_N-term + Desat16_C-term | + |

### Example 16—Production of Z9, E12-14:Acid in Yarrowia lipolytica Using A 12 Desaturase from Ephestia kuehniella

A desaturase of Ephestia kuehniella was co-expressed with the Δ9 desaturase Desat61 in Y. lipolytica (ST12596). Strain ST10748, solely expressing Desat61, served as negative control. The strains were cultivated and FAME were extracted as described in Example 3, with the exception that the cells were incubated for 47 hours in YPG medium instead of 22 hours.

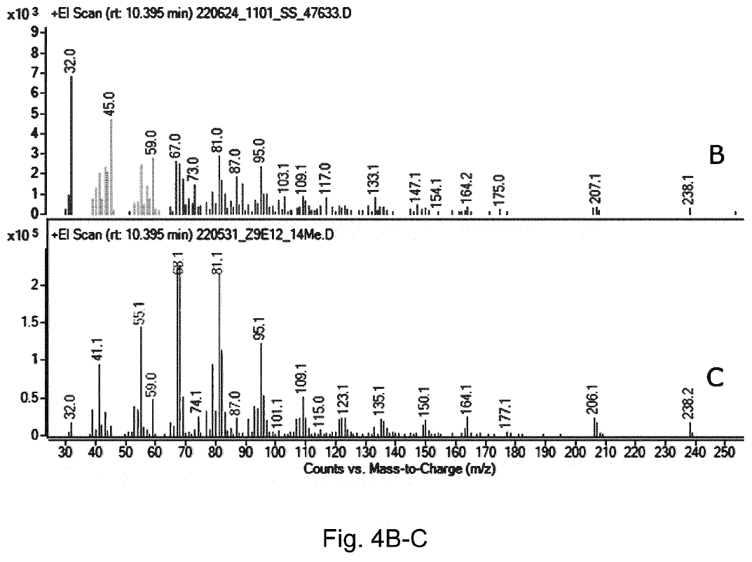

The FAME extract of strain ST12596 contained a double unsaturated methyl tetradecadienoate (FIG. 4A). Both the retention time of 10.39 minutes as well as the mass spectrum of this peak was identical to the once of the pure analytical standard of Z9,E12-14:Me (FIGS. 4A, B and C). The extract of control strain ST10748 did not contain a compound eluting at 10.39 min (FIG. 4A). As the Ephestia kuehniella desaturase was able to introduce a Δ12 double bond, it was given the abbreviation Eku_d12.

### Sequence Overview

| SEQ | | |
|---|---|---|
| ID NO: | Name and accession number | Organism |
| 1 | Pid12 desaturase (DNA) | Plodia interpunctella |
| 2 | Pid12 desaturase (amino acid) | Plodia interpunctella |
| 3 | Desat61 desaturase (DNA) | Drosophila virilis |
| 4 | Desat61 desaturase (amino acid) | Drosophila virilis |
| 5 | Desat24 desaturase (DNA) | Drosophila melanogaster |
| 6 | Desat24 desaturase (amino acid) | Drosophila melanogaster |
| 7 | Desat59 desaturase (DNA) | Drosophila grimshawi |
| 8 | Desat59 desaturase (amino acid) | Drosophila grimshawi |
| 9 | Desat17 desaturase (DNA) | Amyelois transitella |
| 10 | Desat17 desaturase (amino acid) | Amyelois transitella |
| 11 | Desat18 (DNA) | Amyelois transitella |
| 12 | Desat18 (amino acid) | Amyelois transitella |
| 13 | Desat42 (DNA) | Saccharomyces cerevisiae |
| 14 | Desat42 (amino acid) | Saccharomyces cerevisiae |
| 15 | YI_Ole1/Desat69 (DNA) | Yarrowia lipolytica |
| 16 | YI_Ole1/Desat69 (amino acid) | Yarrowia lipolytica |
| 17 | Desat26 (DNA) | Spodoptera litura |
| 18 | Desat26 (amino acid) | Spodoptera litura |
| 19 | FAR1 (DNA) | Helicoverpa armigera |
| 20 | FAR1 (amino acid) | Helicoverpa armigera |
| 21 | FAR 16 (DNA) | Spodoptera exigua |
| 22 | FAR 16 (amino acid) | Spodoptera exigua |
| 23 | FAR17 (DNA) | Spodoptera exigua |
| 24 | FAR17 (amino acid) | Spodoptera exigua |
| 25 | FAR19 (DNA) | Spodoptera litura |
| 26 | FAR19 (amino acid) | Spodoptera litura |
| 27 | FAR28 (DNA) | Plodia interpunctella |
| 28 | FAR28 (amino acid) | Plodia interpunctella |
| 29 | FAR32 (DNA) | Plodia interpunctella |
| 30 | FAR32 (amino acid) | Plodia interpunctella |
| 31 | FAR38 (DNA) | Trichoplusia ni |
| 32 | FAR38 (amino acid) | Trichoplusia ni |
| 33 | PR25093 | Primer |
| 34 | PR25094 | Primer |
| 35 | PR25314 | Primer |
| 36 | PR25381 | Primer |
| 37 | DgNcb5or | Drosophila grimshawi |
| 38 | DmNcb5or | Drosophila melanogaster |
| 39 | HsNcb5or | Homo sapiens |
| 40 | SlitNcb5or | Spodoptera litura |
| 41 | CpoNcb5or1 | Cydia pomonella |
| 42 | FAR29 (DNA) | Plodia interpunctella |
| 43 | FAR29 (amino acid) | Plodia interpunctella |
| 44 | FAR31 (DNA) | Plodia interpunctella |
| 45 | FAR31 (amino acid) | Plodia interpunctella |
| 46 | Desat65 (DNA) | Plodia interpunctella |
| 47 | Desat65 (amino acid) | Plodia interpunctella |
| 48 | Desat66 (DNA) | Plodia interpunctella |
| 49 | Desat66 (amino acid) | Plodia interpunctella |
| 50 | Desat67 (DNA) | Plodia interpunctella |
| 51 | Desat67 (amino acid) | Plodia interpunctella |
| 52 | Desat68 (DNA) | Plodia interpunctella |
| 53 | Desat68 (amino acid) | Plodia interpunctella |
| 54 | EcauDes12 (DNA) | Cadra cautella |
| 55 | EcauDes12 (amino acid) | Cadra cautella |
| 56 | Desat56 (DNA) | Drosophila yakuba |
| 57 | Desat56 (amino acid) | Drosophila yakuba |
| 58 | Desat60 (DNA) | Drosophila ananassae |
| 59 | Desat60 (amino acid) | Drosophila ananassae |
| 60 | Desat74 (DNA) | Choristoneura parallela |
| 61 | Desat74 (amino acid) | Choristoneura parallela |
| 62 | FAR44 (DNA) | Ostrinia furnacalis |
| 63 | FAR44 (amino acid) | Ostrinia furnacalis |
| 64 | FAR48 (DNA) | Ostrinia zag |
| 65 | FAR48 (amino acid) | Ostrinia zag |
| 66 | FAR49 (DNA) | Ostrinia zea |
| 67 | FAR49 (amino acid) | Ostrinia zea |
| 68 | FAR4 (DNA) | Heliothis subflexa |
| 69 | FAR4 (amino acid) | Heliothis subflexa |
| 70 | FAR6 (DNA) | Helicoverpa assulta |
| 71 | FAR6 (amino acid) | Helicoverpa assulta |
| 72 | FAR8 (DNA) | Yponomeuta rorellus |
| 73 | FAR8 (amino acid) | Yponomeuta rorellus |
| 74 | FAR12 (DNA) | Agrotis segetum |
| 75 | FAR12 (amino acid) | Agrotis segetum |
| 76 | FAR11 (DNA) | Bicyclus anynana |
| 77 | FAR11 (amino acid) | Bicyclus anynana |
| 78 | FAR5 (DNA) | Heliothis virescens |
| 79 | FAR5 (amino acid) | Heliothis virescens |
| 80 | Pid12 - Desat65 fusion | — |
| | (amino acid) | |
| 81 | EcauDes12 - Desat65 fusion | — |
| | (amino acid) | |
| 82 | EcauDes12 - Desat16 fusion | — |
| | (amino acid) | |
| 83 | Desat16 (amino acid) | Amyelois transitella |
| 84 | Eku_d12 (DNA) | Ephestia kuehniella |
| 85 | Eku_d12 (amino acid) | Ephestia kuehniella |
| 86 | Ee_d12 (DNA) | Ephestia elutella |
| 87 | Ee_d12 (amino acid) | Ephestia elutella |
| 88 | AseNcb5or (amino acid) | Agrotis segetum |
| 89 | BterNcb5or (amino acid) | Bombus terrestris |
| 90 | LboNcb5or (amino acid) | Lobesia botrana |

### REFERENCES

Alfaro et al. 2009. Optimization of pheromone dispenser density for managing the rice striped stem borer, Chilo suppressalis (Walker), by mating disruption. Crop Protection. 28:567-572.
Bari, 2003. Development of pheromone mating disruption strategies for the suppression of the artichoke plume moth in artichokes grown on the central coast of California. ISHS Acta Horticulturae 660: V International Congress on Artichoke. doi: 10.17660/ActaHortic.2004.660.80 Brady et al. 1971. Sex stimulant and attractant in the Indian meal moth and in the almond moth. Science. 171:802-804.
Deng, B et al. (2010). Study of the individual cytochrome b5 and cytochrome b5 reductase domains of Ncb5or reveals a unique heme pocket and a possible role the CS domain. The Journal of Biological Chemistry, 30181-30191.
Ding, B J et al. (2021). B Bioproduction of (Z,E)-9,12-tetradecadienyl acetate (ZETA), the major pheromone component of Plodia, Ephestia, and Spodoptera species in yeast. Pest Management Science, 78(3), 1048-1059.
Eizaguirre et al. 2002. Effects of mating disruption against the Mediterranean corn borer, Sesamia nonagrioides, on the European corn borer Ostrinia nubilalis. Use of pheromones and other semiochemicals in integrated production IOBC wprs Bulletin.
Holkenbrink et al., 2018. EasyCloneYALI: CRISPR/Cas9-Based Synthetic Toolbox for Engineering of the Yeast Yarrowia lipolytica. Biotechnol J., September; 13(9) Holkenbrink et al., 2020. Production of moth sex pheromones for pest control by yeast fermentation. Metab Eng. 2020 November; 62:312-321.
Jensen et al., 2014. EasyClone: method for iterative chromosomal integration of multiple genes in Saccharomyces cerevisiae. FEMS Yeast Research, 238-248.
Kehat & Dunkelblum, 1993. Sex Pheromones: achievements in monitoring and mating disruption of cotton pests in Israel, Achieves of Insect Biochemistry and Physiology. 22:425-431.
Petkevicius et al., 2021. Biotechnological production of the European corn borer sex pheromone in the yeast Yarrowia lipolytica. Biotechnology J., June; 16(6) Tsakraklides et al. (2018). High-oleate yeast oil without polyunsaturated fatty acids. Biotechnology for biofuels, 11, 131. https://doi.org/10.1186/s13068-018-1131-y
Wakamura et al. (1987). Sex pheromone of the beet armyworm Spodoptera exigua (Hubner) (Lepidoptera: Noctuidae): Field attraction of male moths in Japan to (Z, E)-9 12-tetradecadienylacetate and (Z)-9 tetradecen-1-ol. Applied Entomology and Zoology, 22(3), 348-351.
Wu et al., 2012. Management of diamondback moth, Plutella xylostella (Lepidoptera: Plutellidae) by mating disruption. Insect Science 19 (6), 643-648.
Xia et al., 2019. Multi-Functional Desaturases in Two Spodoptera Moths with Δ11 and Δ12 Desaturation Activities. J Chem Ecol 45, 378-387.
Zhu et al., 1999. Reidentification of the female sex pheromone of the Indian meal moth, Plodia interpunctella: Evidence for a four-component pheromone blend. Entomologia Experimentalis Et Applicata, 92(2), p. 137-146.

### Items

1. A yeast cell capable of producing a desaturated fatty acyl-coenzyme A (fatty acyl-CoA) having n′ double bond(s), wherein at least one of said double bond(s) is at position 12,
   wherein said yeast cell expresses a heterologous Δ12 fatty acyl-CoA desaturase, said desaturase being capable of introducing a double bond at position 12 in a saturated or desaturated fatty acyl-CoA substrate, preferably a desaturated fatty acyl-CoA, having a carbon chain length of at least 13 and having n double bond(s),
   wherein n and n′ are integers,
   wherein 0≤n≤3 and wherein 1≤n′≤4.
2. The yeast cell according to item 1, wherein the saturated or desaturated fatty acyl-CoA used as a substrate and the desaturated fatty acyl-CoA having n′ double bonds have the same carbon chain length.
3. The yeast cell according to any one of the preceding items, wherein said heterologous Δ12 desaturase is native to an organism of a genus selected from the group consisting of Cadra, Ephestia, Plodia, Maliarpha, and Amorbia.
4. The yeast cell according to any one of the preceding items, wherein said heterologous Δ12 desaturase is native to an organism of a species selected from the group consisting of Cadra cautella, Ephestia elutella, Ephestia kuehniella, Plodia interpunctella, Maliarpha separatella, and Amorbia cuneana.
5. The yeast cell according to any one of the preceding items, wherein said heterologous Δ12 desaturase is a Plodia desaturase, such as a Plodia interpunctella desaturase.
6. The yeast cell according to any one of the preceding items, wherein said heterologous Δ12 desaturase is a Cadra desaturase, such as a Cadra cautella desaturase.
7. The yeast cell according to any one of the preceding items, wherein said heterologous Δ12 desaturase is an Ephestia desaturase, such as an Ephestia elutella desaturase or an Ephestia kuehniella desaturase.
8. The yeast cell according to any one of the preceding items, wherein said heterologous Δ12 desaturase is a Pid12 desaturase as set forth in SEQ ID NO: 2 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
9. The yeast cell according to any one of the preceding items, wherein said heterologous Δ12 desaturase is an EcauDes12 desaturase as set forth in SEQ ID NO: 55 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
10. The yeast cell according to any one of the preceding items, wherein said heterologous Δ12 desaturase is an Eku_d12 desaturase as set forth in SEQ ID NO: 85 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
11. The yeast cell according to any one of the preceding items, wherein said heterologous Δ12 desaturase is an Ee_d12 desaturase as set forth in SEQ ID NO: 87 or a functional variant thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
12. The yeast cell according to any one of the preceding items, wherein said heterologous Δ12 desaturase is selected from the desaturases set forth in SEQ ID NO: 80, SEQ ID NO: 81 and SEQ ID NO: 82.
13. The yeast cell according to any one of the preceding items, wherein said saturated or desaturated fatty acyl-CoA having a carbon chain length of at least 13 and having n double bonds, has a carbon length of at least 14, such as at least 15.
14. The yeast cell according to any one of the preceding items, wherein said saturated or desaturated fatty acyl-CoA having a carbon chain length of at least 13 and having n double bonds is tetradecanoyl-CoA or (Z9)-tetradecenoyl-CoA.
15. The yeast cell according to any one of the preceding items, wherein n=1.
16. The yeast cell according to any one of the preceding items, wherein said saturated or desaturated fatty acyl-CoA having a carbon chain length of at least 13 and having n double bonds is (Z9)-tetradecenoyl-CoA.
17. The yeast cell according to any one of the preceding items, wherein said desaturated fatty acyl-CoA having n′ double bond(s) has a carbon chain length of at least 13, such as at least 14, such as at least 15, such as at least 16, such as at least 17, such as at least 18, such as at least 19, such as at least 20.
18. The yeast cell according to any one of the preceding items, wherein said desaturated fatty acyl-CoA having n′ double bond(s) has a carbon chain length of at the most 18, such as at the most 17, such as at the most 16, such as at the most 15, such as at the most 14, such as at least 15, such as at least 16, such as at least 17, such as at least 18, such as at least 19, such as at least 20.
19. The yeast cell according to any one of the preceding items, wherein n′=1.
20. The yeast cell according to any one of the preceding items, wherein said desaturated fatty acyl-CoA having n′ double bond(s) is (E12)-tetradecenoyl-CoA.
21. The yeast cell according to any one of the preceding items, wherein n′=2.
22. The yeast cell according to any one of the preceding items, wherein said desaturated fatty acyl-CoA having n′ double bond(s) is (Z9, E12)-tetradecadienoyl-CoA.
23. The yeast cell according to any one of the preceding items, wherein said yeast cell expresses a further heterologous desaturase.
24. The yeast cell according to any one of the preceding items, wherein said further heterologous desaturase is capable of introducing a double bond at any position which is not position 12 in a saturated or desaturated fatty acyl-CoA having a carbon chain length of at least 13 and having n double bonds.
25. The yeast cell according to any one of the preceding items, wherein said further heterologous desaturase is capable of introducing a double bond at least in position 9.
26. The yeast cell according to any one of the preceding items, wherein said further heterologous desaturase is selected from the group consisting of a Δ9 desaturase and a Δ11 desaturase.
27. The yeast cell according to any one of the preceding items, wherein said further heterologous desaturase has a higher specificity towards tetradecanoyl-CoA or tetradecenoyl-CoA than towards hexadecanoyl-CoA or hexadecanoyl-CoA, respectively.
28. The yeast cell according to any one of the preceding items, wherein said further heterologous desaturase is a Drosophila or a Choristoneura desaturase, such as a Drosophila melanogaster, Drosophila virilis, Drosophila grimshawi, Drosophila yakuba, Drosophila mojavensis, Drosophila pseudoobscura, Drosophila ananassae or a Choristoneura parallela desaturase.
29. The yeast cell according to any one of the preceding items, wherein said further heterologous desaturase is a Drosophila desaturase selected from the group consisting of Desat59 (SEQ ID NO: 8), Desat61 (SEQ ID NO: 4), Desat56 (SEQ ID NO: 57), Desat60 (SEQ ID NO: 59) and Desat24 (SEQ ID NO: 6) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
30. The yeast cell according to any one of the preceding items, wherein said further heterologous desaturase is Choristoneura para/lela desaturase Desat74 (SEQ ID NO: 61).
31. The yeast cell according to any one of the preceding items, wherein the yeast cell is capable of producing (Z9, E12)-tetradecadien-1-ol, said yeast cell further expressing at least one alcohol-forming fatty acyl-CoA reductase (FAR) capable of converting at least a part of (Z9, E12)-tetradecadienoyl-CoA into (Z9, E12)-tetradecadien-1-ol.
32. The yeast cell according to any one of the preceding items, wherein the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, and Yponomeuta, such as Agrotis ipsilon, Agrotis segetum, Amyelois transitella, Bicyclus anynana, Bombus lapidarius, Chilo Suppressalis, Cydia pomonella, Helicoverpa armigera, Helicoverpa assulta, Heliothis subflexa, Heliothis virescens, Lobesia botrana, Ostrinia furnacalis, Ostrinia nubilalis, Ostrinia zag, Ostrinia zea, Plodia interpunctella, Plutella xylostella, Spodoptera exigua, Spodoptera frugiperda, Spodoptera littoralis, Spodoptera litura, Trichoplusia ni, Tyta alba, and Yponomeuta rorellus, especially Spodoptera exigua, Helicoverpa armigera, Spodoptera litura and Plodia interpunctella.
33. The yeast cell according to any one of the preceding items, wherein the FAR is selected from the group consisting of FAR1 (SEQ ID NO: 20), FAR16 (SEQ ID NO: 22), FAR17 (SEQ ID NO: 24), FAR19 (SEQ ID NO: 26), FAR28 (SEQ ID NO: 28) or FAR32 (SEQ ID NO: 30), FAR44 (SEQ ID NO: 63), FAR48 (SEQ ID NO: 65), FAR49 (SEQ ID NO: 67); FAR38 (SEQ ID NO: 32), FAR4 (SEQ ID NO: 69), FAR6 (SEQ ID NO: 71), FAR8 (SEQ ID NO: 73), FAR12 (SEQ ID NO: 75), FAR11 (SEQ ID NO: 77) or FAR5 (SEQ ID NO: 79) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
34. The yeast cell according to any one of the preceding items, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.
35. The yeast cell according to any one of the preceding items, further expressing a heterologous NAD(P)H cytochrome b5 oxidoreductase (Ncb5or).
36. The yeast cell according to any one of the preceding items, wherein the Ncb5or is native to a plant, an insect or a mammal, such as Homo sapiens.
37. The yeast cell according to any one of the preceding items, wherein the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa.
38. The yeast cell according to any one of the preceding items, wherein the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.
39. The yeast according to any one of the preceding items, wherein the Ncb5or is selected from the group of Ncb5ors set forth in SEQ ID NO: 37, SEQ ID NO: 38, SEQ ID NO: 39, SEQ ID NO: 40, SEQ ID NO: 41, SEQ ID NO: 88, SEQ ID NO: 89 or SEQ ID NO: 90, or functional variants having at least 70% identity or similarity thereto, such as at least 75% identity, such as at least 80% identity, such as at least 85% identity, such as at least 90% identity, such as at least 95% identity or similarity thereto.
40. The yeast cell according to any one of the preceding items, having a mutation leading to partial or total loss of activity of one or more of Hfd1, Hfd4, Pex10, GPAT and/or Fao1, such as having at least a mutation leading to partial or total loss of activity of Fao1 and one or more of Hfd1, Hfd4, Pex10 and/or GPAT.
41. The yeast cell according to any one of the preceding items, having a mutation in PEX10 and at least one of HFD1, HFD4, FAO1 and/or GPAT or a homologue thereof.
42. The yeast cell according to any one of the preceding items, having a mutation in FAO1 and at least one of HFD1, HFD4, PEX10 and/or GPAT or a homologue thereof.
43. The yeast cell according to any one of the preceding items, wherein HFD1, HFD4, PEX10 and/or FAO1 or a functional variant thereof having at least 60% identify thereto is deleted or mutated, resulting in partial loss of total loss of activity of Hfd1, Hfd4, Pex10 and/or Fao1, and/or wherein GPAT or a functional variant thereof having at least 60% identity thereto is mutated, resulting in reduced activity of GPAT.
44. The yeast cell according to any one of the preceding items, comprising a mutation in at least one POX gene, such as a POX gene selected from the group consisting of POX1, POX2, POX3, POX4, POX5, and POX6.
45. The yeast cell according to any one of the preceding items, wherein POX1, POX2, POX3, POX4, POX5, and/or POX6 or a functional variant thereof having at least 60% identify thereto is deleted or mutated, resulting in partial loss of total loss of activity of Pox1, Pox2, Pox3, Pox4, Pox5 and/or Pox6.
46. The yeast cell according to any one of the preceding items, wherein said yeast cell further expresses:
   a heterologous cytochrome b5;
   a heterologous cytochrome b5 reductase;
   a hemoglobin;
   a heterologous thioesterase gene; and/or
   a fusion protein of a fatty acyl synthase and of a thioesterase.
47. The yeast cell according to any one of the preceding items, having an inactivation or modification of:
   native elongase(s) resulting in total or partial loss of activity;
   thioesterases(s) resulting in total or partial loss of activity; and/or
   activity of native fatty aldehyde dehydrogenase(s), fatty alcohol oxidase(s), peroxisome biogenesis factor and/or fatty acyl synthase(s).
48. The yeast cell according to any one of the preceding items, wherein the yeast cell is further modified to increase availability of tetradecanoyl-CoA.
49. The yeast cell according to any one of the preceding items, wherein at least one of the genes encoding the heterologous Δ12 desaturase, the further heterologous desaturase or the FAR is present in a high copy number.
50. The yeast cell according to any one of the preceding items, wherein at least one of the genes encoding the heterologous Δ12 desaturase, the further heterologous desaturase or the FAR is under the control of an inducible promoter.
51. The yeast cell according to any one of the preceding items, wherein at least one of the genes encoding the heterologous Δ12 desaturase, the further heterologous desaturase or the FAR are each independently comprised within the genome of the cell or within a vector comprised within the yeast cell.
52. The yeast cell according to any one of the preceding items, wherein the yeast cell is capable of producing (Z9, E12)-tetradecadienoic acid with a titer of at least 1 mg/L, such as at least 1.5 mg/L, such as at least 1.7 mg/L or more.
53. The yeast cell according to any one of the preceding items, wherein the yeast cell is capable of producing an E12-fatty acid and/or (Z9, E12)-tetradecadienoic acid with a titer of at least 20 mg/L, such as at least 50 mg/L, such as at least 60 mg/L, such as at least 70 mg/L, such as at least 80 mg/L, such as at least 90 mg/L, such as at least 100 mg/L, such as at least 150 mg/L, such as at least 200 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.
54. The yeast cell according to any one of the preceding items, wherein the yeast cell is capable of producing an E12-fatty alcohol and/or (Z9, E12)-tetradecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L or more.
55. A method for producing a desaturated fatty acyl-CoA having n′ double bond(s), wherein at least one of said double bond(s) is at position 12, in a yeast cell, said method comprising the steps of:
   i) providing a yeast cell according to any one of items 1 to 54,
   ii) incubating said yeast cell in a medium under conditions allowing expression of a heterologous Δ12 desaturase, said desaturase being capable of introducing a double bond at position 12 in a saturated or desaturated fatty acyl-CoA, preferably a desaturated fatty-acyl-CoA, having a carbon chain length of at least 13 and having n double bonds,
   wherein n and n′ are integers,
   wherein 0≤n≤3 and wherein 1≤n′≤4,
   thereby producing a desaturated fatty acyl-CoA having n′ double bond(s), wherein at least one of said double bond(s) is at position 12.
56. The method according to item 55, wherein said heterologous Δ12 desaturase is as defined in any one of the preceding items.
57. The method according to any one of items 55 to 56, wherein said further heterologous desaturase is as defined in any one of the preceding items.
58. The method according to any one of items 55 to 57, wherein said desaturated fatty acyl-CoA having n′ double bond(s) is as defined in any one of the preceding items.
59. The method according to any one of items 55 to 58, wherein said saturated or desaturated fatty acyl-CoA having a carbon chain length of at least 13 and having n double bonds is as defined in any one of the preceding items.
60. The method according to any one of items 55 to 59, wherein said yeast cell further expresses at least one alcohol-forming fatty acyl-CoA reductase (FAR) capable of converting at least part of (Z9, E12)-tetradecadienoyl-CoA into (Z9, E12)-tetradecadien-1-ol.
61. The method according to any one of items 55 to 60, wherein said FAR is as defined in any one of the preceding items.
62. The method according to any one of items 55 to 61, further comprising the step of formulating the E12-fatty alcohol and/or the (Z9, E12)-tetradecadien-1-ol in a pheromone composition.
63. The method according to any one of items 55 to 62, further comprising the step of recovering the E12-fatty alcohol and/or (Z9, E12)-tetradecadien-1-ol.
64. The method according to any one of items 55 to 63, wherein the E12-fatty alcohol and/or the (Z9, E12)-tetradecadien-1-ol is further converted into an E12-fatty alcohol acetate and/or (Z9, E12)-tetradecadien-1-ol acetate, respectively.
65. The method according to any one of items 55 to 64, wherein the E12-fatty alcohol and/or the (Z9, E12)-tetradecadien-1-ol is further converted into an E12-fatty aldehyde and/or (Z9, E12)-tetradecadienal, respectively.
66. The method according to any one of items 55 to 65, wherein the conversion of the E12-fatty alcohol and/or the (Z9, E12)-tetradecadien-1-ol to an E12-fatty alcohol acetate and/or (Z9, E12)-tetradecadien-1-ol acetate is:
   a. performed in vivo by further expressing in the yeast cell an acetyltransferase capable of converting the E12-fatty alcohol and/or the (Z9, E12)-tetradecadien-1-ol to an E12-fatty alcohol acetate and/or (Z9, E12)-tetradecadien-1-ol acetate, respectively; or
   b. performed in vitro, e.g. by chemical conversion.
67. The method according to any one of items 55 to 66, wherein the E12-fatty alcohol and/or the (Z9, E12)-tetradecadien-1-ol is oxidized into an (E12)-fatty aldehyde and/or (Z9, E12)-tetradecadienal, optionally wherein the oxidation is performed chemically.
68. The method according to any one of items 55 to 66, wherein the E12-fatty alcohol and/or the (Z9, E12)-tetradecadien-1-ol is chemically acetylated into a E12-fatty alcohol acetate and/or (Z9, E12)-tetradecadien-1-ol acetate.
69. A nucleic acid or a system of nucleic acids for modifying a yeast cell, said nucleic acid or system of nucleic acids comprising at least one polynucleotide encoding a heterologous Δ12 desaturase said desaturase being capable of introducing a double bond at position 12 in a saturated or desaturated fatty acyl-CoA having a carbon chain length of at least 13 and having n double bonds thereby producing a desaturated fatty acyl-CoA having n′ double bond(s),
   wherein at least one of said double bond(s) is at position 12,
   wherein n and n′ are integers,
   wherein 0≤n≤3 and wherein 1≤n′≤4.
70. The nucleic acid or system of nucleic acids according to item 69, wherein the heterologous Δ12 desaturase is encoded by any one of the sequences set forth in SEQ ID NO: 1, SEQ ID NO: 54, SEQ ID NO: 84 and SEQ ID NO: 86, or functional variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
71. The nucleic acid or system of nucleic acids according any one of items 69 to 70, wherein said nucleic acid or nucleic acid or system of nucleic acids further comprise a polynucleotide encoding a further heterologous desaturase capable of introducing a double bond at any position which is not position 12 in a saturated or desaturated fatty acyl-CoA having a carbon chain length of at least 13 and having n double bonds.
72. The nucleic acid or system of nucleic acids according to any one of items 67 to 71, wherein said further heterologous desaturase is encoded by any one of the sequences set forth in SEQ ID NO: 3, SEQ ID NO: 5, SEQ ID NO: 7, SEQ ID NO: 56, SEQ ID NO: 58 and SEQ ID NO: 60, or functional variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
73. The nucleic acid or system of nucleic acids according to any one of items 67 to 72, wherein the nucleic acid or system of nucleic acids further comprise a polynucleotide encoding a fatty acyl-CoA reductase (FAR) capable of converting at least a part of the double desaturated (Z9, E12)-tetradecadienoyl-CoA into (Z9, E12)-tetradecadien-1-ol.
74. The nucleic acid or system of nucleic acids according to item 73, wherein the FAR is encoded by any one of the sequences set forth in SEQ ID NO: 19, SEQ ID NO: 21, SEQ ID NO: 23, SEQ ID NO: 25, SEQ ID NO: 27, SEQ ID NO: 29, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76 and SEQ ID NO: 78, or functional variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.
75. A method of monitoring the presence of pest or disrupting the mating of pest, said method comprising the steps of:
   i) producing (Z9, E12)-tetradecadien-1-ol and/or an E12-fatty alcohol by the method according to any one of items 55 to 63;
   ii) converting the E12-fatty alcohol and/or (Z9, E12)-tetradecadien-1-ol into an E12-fatty alcohol acetate and/or (Z9, E12)-tetradecadien-1-ol acetate, respectively;
   iii) converting the E12-fatty alcohol and/or (Z9, E12)-tetradecadien-1-ol into an E12-fatty aldehyde and/or (Z9, E12)-tetradecadienal, respectively;
   iv) formulating said E12-fatty alcohol, (Z9, E12)-tetradecadien-1-ol, E12-fatty alcohol acetate, (Z9, E12)-tetradecadien-1-ol acetate, E12-fatty aldehyde, and/or (Z9, E12)-tetradecadienal as a pheromone composition; and
   v) employing said pheromone composition as an integrated pest management composition.
76. A pheromone composition obtainable by a method comprising the following steps:
   i) producing (Z9, E12)-tetradecadien-1-ol and/or an (E12)-fatty alcohol by the method according to any one of items 55 to 63;
   ii) converting the (E12)-fatty alcohol and/or (Z9, E12)-tetradecadien-1-ol into an (E12)-fatty alcohol acetate and/or (Z9, E12)-tetradecadien-1-ol acetate, respectively;
   iii) converting the (E12)-fatty alcohol and/or (Z9, E12)-tetradecadien-1-ol into an (E12)-fatty aldehyde and/or (Z9, E12)-tetradecadienal, respectively;
   iv) formulating said (E12)-fatty alcohol, (Z9, E12)-tetradecadien-1-ol, E12-fatty alcohol acetate, (Z9, E12)-tetradecadien-1-ol acetate, (E12)-fatty aldehyde, and/or (Z9, E12)-tetradecadienal as a pheromone composition.
77. A pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein said pheromone composition comprises at least 20% biobased carbon.
78. A pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein said pheromone compound comprises at least 20% biobased carbon.
79. The pheromone composition according to item 77, further comprising one or more additional compounds selected from the group consisting of liquid carriers, solid carriers and substrates.
80. The pheromone composition according to any of items 77 or 79, further comprising one or more compounds selected from the group consisting of vegetable oils, refined mineral oils or fractions thereof, rubbers, plastics, silica, diatomaceous earth, wax matrix and cellulose powder.
81. The pheromone composition according to any of items 77 or 79-80, wherein said pheromone composition is in the form of a solution, a gel, or a powder.
82. The pheromone composition according to any of items 77 or 79-81, wherein said pheromone composition is formulated so that it can be dispensed.
83. The pheromone composition according to any of items 77 or 79-82, wherein the ratio of (Z9, E12)-tetradecadien-1-ol to the E12-fatty alcohol, (Z9, E12)-tetradecadien-1-ol acetate to the E12-fatty alcohol acetate and/or (Z9, E12)-tetradecadienal to the E12-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
84. The pheromone composition according to any of items 77 or 79-83, wherein the ratio of (Z9, E12)-tetradecadien-1-ol to (Z9, E12)-tetradecadienal and/or (Z9, E12)-tetradecadien-1-ol to (Z9, E12)-tetradecadien-1-ol acetate in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
85. The pheromone composition according to any of items 77 or 79-84, wherein the ratio of (Z9, E12)-tetradecadien-1-ol to a Z9-fatty alcohol, (Z9, E12)-tetradecadien-1-ol acetate to a Z9-fatty alcohol acetate and/or (Z9, E12)-tetradecadienal to a Z9-fatty aldehyde in the pheromone composition is of at least 0.1, such as at least 0.15, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.
86. The pheromone composition or the pheromone compound according to any of items 77-85, wherein said pheromone composition or said pheromone compound comprises at least 30% biobased carbon, alternatively 40% biobased carbon, alternatively 50% biobased carbon, alternatively 60% biobased carbon, alternatively 70% biobased carbon, alternatively 75% biobased carbon, alternatively 80% biobased carbon, alternatively 85% biobased carbon, alternatively 90% biobased carbon, alternatively 95% biobased carbon.
87. The pheromone composition or the pheromone compound according to any of items 77-86, wherein said pheromone composition or said pheromone compound comprises at least 80% biobased carbon, alternatively 85% biobased carbon, alternatively 90% biobased carbon, alternatively 95% biobased carbon, alternatively 96% biobased carbon, alternatively 97% biobased carbon, alternatively 98% biobased carbon, alternatively 99% biobased carbon.
88. The pheromone composition or the pheromone compound according to any of items 77-87, wherein said pheromone composition or said pheromone compound comprises 30-100% biobased carbon, alternatively 40-100% biobased carbon, alternatively 50-100% biobased carbon, alternatively 60-100% biobased carbon, alternatively 70-100% biobased carbon, alternatively 75-100% biobased carbon, alternatively 80-100% biobased carbon, alternatively 85-100% biobased carbon, alternatively 90-100% biobased carbon, alternatively 95-100% biobased carbon.
89. The pheromone composition or the pheromone compound according to any of items 77-88, wherein said pheromone composition or said pheromone compound comprises 80-100% biobased carbon, alternatively 85-100% biobased carbon, alternatively 90-100% biobased carbon, alternatively 95-100% biobased carbon, alternatively 96-100% biobased carbon, alternatively 97-100% biobased carbon, alternatively 98-100% biobased carbon, alternatively 99-100% biobased carbon.
90. A method for producing a pheromone composition according to any of items 77 or 79-89 comprising the following steps:
   i) producing (Z9, E12)-tetradecadien-1-ol and/or an (E12)-fatty alcohol by the method according to any one of items 55 to 63;
   ii) converting the (E12)-fatty alcohol and/or (Z9, E12)-tetradecadien-1-ol into an (E12)-fatty alcohol acetate and/or (Z9, E12)-tetradecadien-1-ol acetate, respectively;
   iii) converting the (E12)-fatty alcohol and/or (Z9, E12)-tetradecadien-1-ol into an (E12)-fatty aldehyde and/or (Z9, E12)-tetradecadienal, respectively;
   iv) formulating said (E12)-fatty alcohol, (Z9, E12)-tetradecadien-1-ol, E12-fatty alcohol acetate, (Z9, E12)-tetradecadien-1-ol acetate, (E12)-fatty aldehyde, and/or (Z9, E12)-tetradecadienal as a pheromone composition, optionally comprising one or more additional compounds such as a liquid or solid carrier or substrate;
   v) optionally determining the biobased carbon content of the pheromone composition.
91. A method for producing a pheromone compound according to any of items 78-89, in a yeast cell, said method comprising the steps of:
   i) providing a yeast cell expressing a heterologous Δ12 fatty acyl-CoA desaturase,
   ii) incubating said yeast cell in a medium under conditions allowing expression of a heterologous Δ12 fatty acyl-CoA desaturase,
thereby producing said pheromone compound.
92. A yeast cell capable of producing a pheromone compound according to any of items 78-89, wherein said yeast cell expresses a heterologous Δ12 fatty acyl-CoA desaturase.
93. The method or the yeast cell according to any of items 91-92, wherein said yeast cell expresses a further heterologous desaturase.
94. The method or the yeast cell according to any of items 91-93, wherein said yeast cell further expresses at least one alcohol-forming fatty acyl-CoA reductase (FAR).
95. The method according to any of items 91-94 further comprising a step of determining the biobased carbon content of the pheromone compound.
96. Use of a Plodia interpunctella desaturase in a method for introducing a double bond at position 12 in a saturated or desaturated fatty acyl-CoA.
97. The use according to item 96, wherein the desaturated fatty acyl-CoA has a carbon length of 12 or more, such as 13, such as 14, such as 15 or more and/or the saturated fatty acid methyl ester has a carbon length of 12 or more, such as 13, such as 14, such as 15 or more.
98. The use according to any one of items 96 to 97, wherein the Plodia interpunctella desaturase is Pid12 (SEQ ID NO: 2) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
99. Use of a Cadra cautella desaturase in a method for introducing a double bond at position 12 in a saturated or desaturated fatty acyl-CoA.
100. The use according to item 99, wherein the desaturated fatty acyl-CoA has a carbon length of 12 or more, such as 13, such as 14, such as 15 or more and/or the saturated fatty acid methyl ester has a carbon length of 12 or more, such as 13, such as 14, such as 15 or more.
101. The use according to any one of items 99 to 100, wherein the Cadra cautella desaturase is EcauDes12 (SEQ ID NO: 55) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
102. Use of an Ephestia kuehniella desaturase in a method for introducing a double bond at position 12 in a saturated or desaturated fatty acyl-CoA.
103. The use according to item 102, wherein the desaturated fatty acyl-CoA has a carbon length of 12 or more, such as 13, such as 14, such as 15 or more and/or the saturated fatty acid methyl ester has a carbon length of 12 or more, such as 13, such as 14, such as 15 or more.
104. The use according to any one of items 102 to 103, wherein the Ephestia kuehniella desaturase is Eku_d12 (SEQ ID NO: 85) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
105. Use of an Ephestia elutella desaturase in a method for introducing a double bond at position 12 in a saturated or desaturated fatty acyl-CoA.
106. The use according to item 105, wherein the desaturated fatty acyl-CoA has a carbon length of 12 or more, such as 13, such as 14, such as 15 or more and/or the saturated fatty acid methyl ester has a carbon length of 12 or more, such as 13, such as 14, such as 15 or more.
107. The use according to any one of items 105 to 106, wherein the Ephestia elutella desaturase is Ee_d12 (SEQ ID NO: 87) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
108. The use according to any one of items 96 to 101, wherein the method is performed in vitro.
109. The use according to any one of items 96 to 108, wherein the method is performed in vivo, preferably wherein the method is performed in a yeast cell according to any one of items 1 to 54.
110. A (Z9, E12)-tetradecadienoyl-CoA obtainable by the method according to any one of items 55 to 64.
111. A (Z9, E12)-tetradecadien-1-ol obtainable by the method according to any one of items 60 to 64.
112. A fermentation broth comprising the yeast cell according to any one of items 1 to 54.
113. A yeast cell capable of producing (Z9, E12)-tetradecadienoyl-CoA, said yeast cell expressing
   i) at least one first heterologous desaturase capable of introducing a first double bond at position 9 in a tetradecanoyl-CoA, thereby converting said tetradecanoyl-CoA to (Z9)-tetradecenoyl-CoA; and
   ii) at least one second heterologous desaturase capable of introducing a second double bond at position 12 in (Z9)-tetradecenoyl-CoA, thereby converting (Z9)-tetradecenoyl-CoA into (Z9, E12)-tetradecadienoyl-CoA.
114. A yeast cell capable of producing a desaturated fatty acyl-CoA, said yeast cell expressing a Pid12 (SEQ ID NO: 2) desaturase, an EcauDes12 (SEQ ID NO: 55) desaturase, an Eku_d12 (SEQ ID NO: 85) or an Ee_d12 (SEQ ID NO: 87) desaturase capable of introducing a trans-double bond at position 12 in a fatty acyl-CoA, thereby converting the fatty acyl-CoA into an E12-fatty acyl-CoA.
115. A method for producing (Z9, E12)-tetradecadien-1-ol, said method comprising the steps of:
   i) providing a yeast cell according to any one of items 1 to 54,
   ii) incubating said yeast cell in a medium under conditions allowing expression of:
      a. a further desaturase, said desaturase being capable of introducing a double bond at position 9 in tetradecanoyl-CoA, thereby converting at least part of tetradecanoyl-CoA to (Z9)-tetradecenoyl-CoA;
      b. a heterologous Δ12 desaturase, said desaturase being capable of introducing a double bond at position 12 in (Z9)-tetradecenoyl-CoA, thereby converting at least part of (Z9)-tetradecenoyl-CoA to (Z9, E12)-tetradecadienoyl-CoA;
      c. a FAR, said FAR being capable of converting at least part of (Z9, E12)-tetradecadienoyl-CoA into (Z9, E12)-tetradecadien-1-ol;
   iii) optionally, recovering the (Z9, E12)-tetradecadien-1-ol;
   iv) optionally, converting the (Z9, E12)-tetradecadien-1-ol into (Z9, E12)-tetradecadien-1-ol acetate;
   v) optionally, converting the (Z9, E12)-tetradecadien-1-ol into (Z9, E12)-tetradecadienal;
   vi) optionally, formulating the (Z9, E12)-tetradecadien-1-ol, the (Z9, E12)-tetradecadien-1-ol acetate, and/or the (Z9, E12)-tetradecadienal into a pheromone composition.

1. A yeast cell capable of producing a desaturated fatty acyl-coenzyme A (fatty acyl-CoA) having n′ double bond(s), wherein at least one of said double bond(s) is at position 12,
wherein said yeast cell expresses a heterologous Δ12 fatty acyl-CoA desaturase, said desaturase being capable of introducing a double bond at position 12 in a saturated or desaturated fatty acyl-CoA, preferably a desaturated fatty acyl-CoA, having a carbon chain length of at least 13 and having n double bond(s),
wherein n and n′ are integers,
wherein 0≤n≤3 and wherein 1≤n′≤4.

2. The yeast cell according to claim 1, wherein said heterologous Δ12 desaturase is native to an organism of a genus selected from the group consisting of Cadra, Ephestia, Plodia, Maliarpha, Fumibotys and Amorbia, such as to a species selected from the group consisting of Cadra cautella, Ephestia elutella, Ephestia kuehniella, Plodia interpunctella, Maliarpha separatella, and Amorbia cuneana, optionally wherein said heterologous Δ12 desaturase is a Plodia desaturase, such as a Plodia interpunctella desaturase or a Cadra desaturase, such as a Cadra cautella desaturase, or an Ephestia desaturase, such as an Ephestia elutella or an Ephestia kuehniella desaturase, further optionally wherein said heterologous Δ12 desaturase is a Pid12 desaturase as set forth in SEQ ID NO: 2 or an Eku_d12 desaturase as set forth in SEQ ID NO: 85, or a functional homologue thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

3. The yeast cell according to any one of the preceding claims, wherein said heterologous Δ12 desaturase is selected from the desaturases set forth in SEQ ID NO: 80, SEQ ID NO: 81 and SEQ ID NO: 82.

4. The yeast cell according to any one of the preceding claims, wherein said saturated or desaturated fatty acyl-CoA having a carbon chain length of at least 13 and having n double bonds:
i) has a carbon length of at least 14, such as at least 15;
ii) is tetradecanoyl-CoA or tetradecenoyl-CoA;
iii) has one double bond (i.e. n=1); and/or
iv) is (Z9)-tetradecenoyl-CoA.

5. The yeast cell according to any one of the preceding claims, wherein said desaturated fatty acyl-CoA having n′ double bond(s):
i) has a carbon chain length of at the most 18, such as at the most 17, such as at the most 16, such as at the most 15, such as at the most 14, such as at least 15, such as at least 16, such as at least 17, such as at least 18, such as at least 19, such as at least 20;
ii) has one double bond (i.e. n′=1);
iii) has two double bonds (i.e. n′=2);
iv) is (E12)-tetradecenoyl-CoA; and/or
v) is (Z9, E12)-tetradecadienoyl-CoA.

6. The yeast cell according to any one of the preceding claims, wherein said yeast cell expresses a further heterologous desaturase; optionally wherein said further heterologous desaturase is capable of introducing a double bond at any position which is not position 12 in a saturated or desaturated fatty acyl-CoA having a carbon chain length of at least 13 and having n double bonds; optionally wherein said further heterologous desaturase is capable of introducing a double bond at least in position 9; further optionally wherein said further heterologous desaturase is selected from the group consisting of a Δ9 desaturase and a Δ11 desaturase.

7. The yeast cell according to claim 6, wherein said further heterologous desaturase is a Drosophila or a Choristoneura desaturase, such as a Drosophila melanogaster, Drosophila virilis, Drosophila grimshawi, Drosophila yakuba, Drosophila mojavensis, Drosophila pseudoobscura, Drosophila ananassae or a Choristoneura parallela desaturase, optionally wherein said further heterologous desaturase is selected from the group consisting of Desat59 (SEQ ID NO: 8), Desat61 (SEQ ID NO: 4), Desat56 (SEQ ID NO: 57), Desat60 (SEQ ID NO: 59), Desat74 (SEQ ID NO: 61) and Desat24 (SEQ ID NO: 6) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

8. The yeast cell according to any one of the preceding claims, wherein the yeast cell is capable of producing (Z9, E12)-tetradecadien-1-ol, said yeast cell further expressing at least one alcohol-forming fatty acyl-CoA reductase (FAR) capable of converting at least a part of (Z9, E12)-tetradecadienoyl-CoA into (Z9, E12)-tetradecadien-1-ol; optionally wherein the FAR is native to an organism of a genus selected from the group consisting of Agrotis, Amyelois, Bicyclus, Bombus, Chilo, Cydia, Helicoverpa, Heliothis, Lobesia, Ostrinia, Plodia, Plutella, Spodoptera, Trichoplusia, Tyta, and Yponomeuta, such as Agrotis ipsilon, Agrotis segetum, Amyelois transitella, Bicyclus anynana, Bombus lapidarius, Chilo Suppressalis, Cydia pomonella, Helicoverpa armigera, Helicoverpa assulta, Heliothis subflexa, Heliothis virescens, Lobesia botrana, Ostrinia furnacalis, Ostrinia nubilalis, Ostrinia zag, Ostrinia zea, Plodia interpunctella, Plutella xylostella, Spodoptera exigua, Spodoptera frugiperda, Spodoptera littoralis, Spodoptera litura, Trichoplusia ni, Tyta alba, and Yponomeuta rorellus, especially Spodoptera exigua, Helicoverpa armigera, Spodoptera litura and Plodia interpunctella; further optionally wherein the FAR is selected from the group consisting of FAR1 (SEQ ID NO: 20), FAR16 (SEQ ID NO: 22), FAR17 (SEQ ID NO: 24), FAR19 (SEQ ID NO: 26), FAR28 (SEQ ID NO: 28), FAR32 (SEQ ID NO: 30), FAR44 (SEQ ID NO: 63), FAR48 (SEQ ID NO: 65), FAR49 (SEQ ID NO: 67); FAR38 (SEQ ID NO: 32), FAR4 (SEQ ID NO: 69), FAR6 (SEQ ID NO: 71), FAR8 (SEQ ID NO: 73), FAR12 (SEQ ID NO: 75), FAR11 (SEQ ID NO: 77) or FAR5 (SEQ ID NO: 79), or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

9. The yeast cell according to any one of the preceding claims, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast cell is a Yarrowia lipolytica cell or a Saccharomyces cerevisiae cell.

10. The yeast cell according to any one of the preceding claims, wherein the yeast cell is capable of producing:
i) (Z9, E12)-tetradecadienoic acid with a titer of at least 1 mg/L, such as at least 1.5 mg/L, such as at least 1.7 mg/L or more;
ii) an E12-fatty acid and/or (Z9, E12)-tetradecadienoic acid with a titer of at least 20 mg/L, such as at least 50 mg/L, such as at least 60 mg/L, such as at least 70 mg/L, such as at least 80 mg/L, such as at least 90 mg/L, such as at least 100 mg/L, such as at least 150 mg/L, such as at least 200 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more; and/or
iii) an E12-fatty alcohol and/or (Z9, E12)-tetradecadien-1-ol with a titer of at least 0.005 mg/L, such as at least 0.01 mg/L, such as at least 0.02 mg/L, such as at least 0.03 mg/L, such as at least 0.05 mg/L, such as at least 0.06 mg/L, such as at least 0.075 mg/L, such as at least 0.1 mg/L, such as at least 0.2 mg/L, such as at least 0.3 mg/L, such as at least 0.4 mg/L, such as at least 0.5 mg/L, such as at least 0.6 mg/L, such as at least 0.7 mg/L, such as at least 0.8 mg/L, such as at least 0.9 mg/L, such as at least 1 mg/L, such as at least 2 mg/L, such as at least 3 mg/L, such as at least 4 mg/L, such as at least 5 mg/L, such as at least 6 mg/L, such as at least 7 mg/L, such as at least 8 mg/L, such as at least 9 mg/L, such as at least 10 mg/L or more.

11. A method for producing a desaturated fatty acyl-CoA having n′ double bond(s), wherein at least one of said double bond(s) is at position 12, in a yeast cell, said method comprising the steps of:
i) providing a yeast cell according to any one of claims 1 and 10,
ii) incubating said yeast cell in a medium under conditions allowing expression of a heterologous Δ12 desaturase, said desaturase being capable of introducing a double bond at position 12 in a saturated or desaturated fatty acyl-CoA, preferably a desaturated fatty-acyl-CoA, having a carbon chain length of at least 13 and having n double bonds,
wherein n and n′ are integers,
wherein 0≤n≤3 and wherein 1≤n′≤4,
thereby producing a desaturated fatty acyl-CoA having n′ double bond(s),
wherein at least one of said double bond(s) is at position 12.

12. The method according to claim 11, wherein:
i) said heterologous Δ12 desaturase is as defined in claim 2;
ii) said further heterologous desaturase is as defined in any one of claims 6 and 7;
iii) said yeast cell is as defined in any one of claims 1 and 10;
iv) said desaturated fatty acyl-CoA having n′ double bond(s) is as defined in claim 5; and/or
v) said saturated or desaturated fatty acyl-CoA having a carbon chain length of at least 13 and having n double bonds is as defined in claim 4.

13. The method according to any one of claims 11 to 12, wherein said yeast cell further expresses at least one alcohol-forming fatty acyl-CoA reductase (FAR) capable of converting at least part of (E12)-tetradecenoyl-CoA and/or (Z9, E12)-tetradecadienoyl-CoA into an E12-fatty alcohol and/or (Z9, E12)-tetradecadien-1-ol, respectively, optionally wherein said FAR is as defined in claim 8.

14. The method according to any one of claims 11 to 13, further comprising the steps of:
i) recovering the E12-fatty alcohol and/or (Z9, E12)-tetradecadien-1-ol;
ii) converting the E12-fatty alcohol and/or (Z9,E12)-tetradecadien-1-ol into an E12-fatty alcohol acetate and/or (Z9,E12)-tetradecadien-1-ol acetate, respectively;
iii) converting the E12-fatty alcohol and/or (Z9, E12)-tetradecadien-1-ol into an E12-fatty aldehyde and/or (Z9, E12)-tetradecadienal, respectively, and/or
iv) formulating E12-fatty alcohol, (Z9, E12)-tetradecadien-1-ol, E12-fatty alcohol acetate, (Z9,E12)-tetradecadien-1-ol acetate, E12-fatty aldehyde, and/or (Z9, E12)-tetradecadienal in a pheromone composition.

15. A pheromone composition obtainable by a method comprising the following steps:
i) producing (Z9, E12)-tetradecadien-1-ol and/or an E12-fatty alcohol by the method according to any one of claims 11 to 14;
ii) recovering the (Z9, E12)-tetradecadien-1-ol and/or E12-fatty alcohol;
iii) converting the E12-fatty alcohol and/or (Z9, E12)-tetradecadien-1-ol into an E12-fatty alcohol acetate and/or (Z9, E12)-tetradecadien-1-ol acetate, respectively;
iv) converting the E12-fatty alcohol and/or (Z9, E12)-tetradecadien-1-ol into an E12-fatty aldehyde and/or (Z9, E12)-tetradecadienal, respectively;
v) formulating said E12-fatty alcohol, (Z9, E12)-tetradecadien-1-ol, E12-fatty alcohol acetate, (Z9, E12)-tetradecadien-1-ol acetate, E12-fatty aldehyde, and/or (Z9, E12)-tetradecadienal as a pheromone composition.

16. A pheromone composition comprising an E12-fatty alcohol, (Z9, E12)-tetradecadien-1-ol, an E12-fatty alcohol acetate, (Z9, E12)-tetradecadien-1-ol acetate, an E12-fatty aldehyde, and/or (Z9, E12)-tetradecadienal, wherein said pheromone composition comprises at least 70% biobased carbon, such as at least 75% biobased carbon, such as at least 80% biobased carbon, such as at least 85% biobased carbon, such as at least 90% biobased carbon, such as at least 95% biobased carbon.

17. A pheromone compound selected from the group consisting of an E12-fatty alcohol, (Z9, E12)-tetradecadien-1-ol, an E12-fatty alcohol acetate, (Z9, E12)-tetradecadien-1-ol acetate, an E12-fatty aldehyde, and (Z9, E12)-tetradecadienal, wherein said pheromone compound comprises at least 70% biobased carbon, such as at least 75% biobased carbon, such as at least 80% biobased carbon, such as at least 85% biobased carbon, such as at least 90% biobased carbon, such as at least 95% biobased carbon.

18. Use of a Plodia interpunctella desaturase in a method for introducing a double bond at position 12 in a saturated or desaturated fatty acyl-CoA, optionally wherein the Plodia interpunctella desaturase is Pid12 (SEQ ID NO: 2) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.

19. Use of an Ephestia kuehniella desaturase in a method for introducing a double bond at position 12 in a saturated or desaturated fatty acyl-CoA, optionally wherein the Ephestia kuehniella desaturase is Eku_d12 (SEQ ID NO: 85) or functional variants thereof having at least 70% similarity or identity thereto, such as at least 80%, such as at least 90%, such as at least 91%, such as at least 92%, such as at least 93%, such as at least 94%, such as at least 95%, such as at least 96%, such as at least 97%, such as at least 98%, such as at least 99%, such as 100% similarity or identity thereto.
